(19) United States
(12) Patent Application Publication
(10) Pub. No.: US 2017/0258645 A1
(43) Pub. Date: Sep. 14, 2017

(54) THREE-DIMENSIONAL MATERIALS HAVING APERTURES

(71) Applicant: The Procter & Gamble Company, Cincinnati, OH (US)

(72) Inventors: Jill Marlene ORR, Liberty Township, OH (US); Rodrigo ROSATI, Frankfurt Am Main (DE); John Brian STRUBE, Okeana, OH (US); James T. KNAPMEYER, Cincinnati, OH (US); Adrien GRENIER, Frankfurt Am Main (DE)

(21) Appl. No.: 15/453,935

(22) Filed: Mar. 9, 2017

Related U.S. Application Data
(60) Provisional application No. 62/306,877, filed on Mar. 11, 2016.

(51) Int. Cl. A61F 13/512; D06C 23/04; D04H 3/007; A61F 13/538; B32B 3/26; B32B 5/02; B32B 5/26; D04H 3/16; B32B 3/30

(52) U.S. Cl. CPC A61F 13/512; D04H 3/16; D06C 23/04; D04H 3/007; B32B 3/30; B32B 3/266; B32B 5/022; B32B 5/26; A61F 13/538; D10B 2509/026; B32B 2250/20; B32B 2262/0253; B32B 2307/726; B32B 2555/02

(57) ABSTRACT

The present disclosure provides three-dimensional materials having apertures. The apertures in the three-dimensional materials may be formed in portions of a generally planar region and/or in at least some of a plurality of discrete integral second regions. The apertures may help in BM or other bodily fluid acquisition and retention.

### CROSS REFERENCE TO RELATED APPLICATION

This application claims the benefit, under 35 U.S.C. §119(e), to U.S. Provisional Patent Application No. 62/306,877 filed on Mar. 11, 2016, the entire disclosure of which is hereby incorporated by reference.

### FIELD

The present disclosure is directed to three-dimensional materials having apertures. The present disclosure is also directed to absorbent articles comprising three-dimensional nonwoven materials having apertures.

### BACKGROUND

A need exists for improved materials and improved materials for use in absorbent articles. In certain instances, a need exists for improved nonwoven materials or laminates of nonwoven materials or laminates comprising nonwoven materials that look and feel soft, have improved dryness, and have improved bowel movement (“BM”), or other bodily fluid, absorbency, retention, and reduced run-off. In particular, a need exists for improved nonwoven materials having three-dimensional features formed therein to provide improved softness, dryness, and BM, or other bodily fluid, absorbency, retention, and reduced run-off, as well as providing visual signals of softness, dryness, and BM, or other bodily fluid, absorbency, retention, and reduced run-off.

### SUMMARY

The present disclosure provides improved three-dimensional nonwoven materials having improved softness, dryness, and BM, or other bodily fluid, absorbency, retention, and reduced run-off, as well as a visual signal of the same. The three-dimensional nonwoven materials may comprise apertures and create significant void volume for better absorbency, retention, and reduced run-off of BM and other bodily fluids. The apertures allow BM, and the other bodily fluids, to quickly penetrate into absorbent articles, while the increased void volumes allow for better BM, or other bodily fluid, retention. Further, the increased void volumes reduce the spread of BM, and other bodily fluids, once captured, thereby providing reduce run-off benefits. Additionally, the three-dimensional materials of the present disclosure may act to wipe BM, or other bodily fluids, off of skin of a wearer, during wearer movement. Lastly, the three-dimensional nonwoven materials of the present disclosure provide high surface areas and contact with the skin, to entangle BM, or other bodily fluids, and at least reduce BM, or other bodily fluids, from sticking in the skin.

The present disclosure is directed, in part, to a nonwoven material having a first surface and a second surface. The nonwoven material comprises a plurality of fibers, a generally planar first region, and a plurality of discrete integral second regions that comprise deformations forming protrusions extending outwardly from the first surface of the nonwoven material and openings in the second surface of the nonwoven material. The protrusions are formed from the fibers. The protrusions comprise a base proximate to the first surface of the nonwoven material, an opposed distal end extending outward in the Z-direction from the base, side walls between the base and the distal end of the protrusion, and a cap comprising at least a portion of the side walls and the distal end of the protrusions. The side walls have interior surfaces. Multiple fibers extend from the base of the protrusions to the distal end of the protrusions, and contribute to form a portion of the sides and cap of a protrusion. The fibers at least substantially surround the sides of the protrusions. The interior surfaces of the side walls define a base opening at the base of the protrusion. The cap has a portion with a maximum interior width. The base opening has a width. The maximum interior width of the cap of the protrusions is greater than the width of the base opening. A plurality of apertures are defined through the nonwoven material in portions of the generally planar first region or through portions of at least some of the plurality of discrete integral second regions. The apertures are formed in a predetermined, intentional pattern.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a photomicrograph showing the end view of a prior art tuft;

FIG. 2 is a schematic end view of a prior art tuft after it has been subjected to compression;

FIG. 3 is a photomicrograph of the end of a prior art nonwoven web showing a plurality of collapsed tufts;

FIG. 4 is a schematic side view of a prior art conical-shaped structure before and after it has been subjected to compression;

FIG. 5 is a plan view photomicrograph showing one side of the nonwoven material having three-dimensional deformations formed therein, with the protrusions oriented upward;

FIG. 6 is a plan view photomicrograph showing the other side of a nonwoven material similar to that shown in FIG. 5, with the openings in the nonwoven facing upward;

FIG. 7 is a Micro CT scan image showing a perspective view of a protrusion in a single layer nonwoven material;

FIG. 8 is a Micro CT scan image showing a side of a protrusion in a single layer nonwoven material;

FIG. 9 is a Micro CT scan image showing a perspective view of a deformation with the opening facing upward in a single layer nonwoven material;

FIG. 10 is a perspective view of a deformation in a two layer nonwoven material with the opening facing upward;

FIG. 11 is a photomicrograph of a cross-section taken along the transverse axis of a deformation showing one example of a multi-layer nonwoven material having a three-dimensional deformation in the form of a protrusion on one side of the material that provides a wide opening on the other side of the material, with the opening facing upward;

FIG. 12 is a schematic view of the protrusion shown in FIG. 11;

FIG. 13 is a plan view photomicrograph from the protrusion side of a material after it has been subjected to compression showing the high fiber concentration region around the perimeter of the protrusion;

FIG. 14 is a photomicrograph of the cross-section of a protrusion taken along the transverse axis of the protrusion showing the protrusion after it has been subjected to compression;

FIG. 15A is a cross-sectional view taken along the transverse axis of a deformation of one embodiment of a multi-layer nonwoven web shown with the base opening facing upward;

FIG. 15B is a cross-sectional view taken along the transverse axis of a deformation of an alternative embodiment of a multi-layer nonwoven web shown with the base opening facing upward;

FIG. 15C is a cross-sectional view taken along the transverse axis of a deformation of an alternative embodiment of a multi-layer nonwoven web shown with the base opening facing upward;

FIG. 15D is a cross-sectional view taken along the transverse axis of a deformation of an alternative embodiment of a multi-layer nonwoven web shown with the base opening facing upward;

FIG. 15E is a cross-sectional view taken along the transverse axis of a deformation of an alternative embodiment of a multi-layer nonwoven web shown with the base opening facing upward;

FIG. 15F is a cross-sectional view taken along the transverse axis of a deformation of an alternative embodiment of a multi-layer nonwoven web shown with the base opening facing upward;

FIG. 16 is a plan view photomicrograph of a nonwoven web with the protrusions oriented upward showing the concentration of fibers in one layer of a two layer structure;

FIG. 17 is a perspective view photomicrograph showing the reduced fiber concentration in the side walls of the protrusions in a layer similar to that shown in FIG. 16;

FIG. 18 is a plan view photomicrograph of a nonwoven web with the protrusions oriented upward showing the reduced concentration of fibers in the cap of a protrusion in the other layer (i.e. vs. the layer shown in FIG. 16) of a two layer structure;

FIG. 19 is a perspective view photomicrograph showing the decreased fiber concentration in the side walls of the protrusions in a layer similar to that shown in FIG. 18;

FIG. 19A is a Micro CT scan image showing the side of a protrusion in a single layer of nonwoven material with the protrusion oriented downward;

FIG. 19B is a Micro CT scan plan view image showing the base opening of a deformation in a single layer of nonwoven material;

FIG. 20 is a perspective view photomicrograph of one layer of a multiple layer nonwoven material on the surface of a forming roll showing the “hanging chads” that can be formed in one of the layers when some nonwoven precursor web materials are used;

FIG. 21 is a perspective view of one example of an apparatus for forming the nonwoven material described herein;

FIG. 22 is an enlarged perspective view of a portion of the male roll shown in FIG. 21;

FIG. 22A is an enlarged schematic side view showing an example of a surface texture formed by knurling a forming member;

FIG. 22A is a schematic side view of a male element with tapered side walls;

FIG. 22B is a schematic side view of a male element with undercut side walls;

FIG. 22C is an enlarged perspective view of a portion of a male roll having an alternative configuration;

FIG. 22D is a schematic side view of a male element with a rounded top;

FIG. 23 is an enlarged perspective view showing the nip between the rolls shown in FIG. 21;

FIG. 24 is a schematic perspective view of one version of a method of making nonwoven materials having deformations therein where two precursor materials are used, one of which is a continuous web and the other of which is in the form of discrete pieces;

FIG. 24A is a schematic side view of an apparatus for forming the nonwoven material in which the web wraps around one of the rolls before and after passing through the nip between the rolls;

FIG. 25 is an absorbent article in the form of a diaper comprising an exemplary topsheet/acquisition layer composite structure wherein the length of the acquisition layer is less that the length of the topsheet with some layers partially removed;

FIG. 26 is one transverse cross-section of the diaper of FIG. 25 taken along line 26-26;

FIG. 27 is an alternative transverse cross-section of the diaper of FIG. 25;

FIG. 28 is a photograph of a two layer apertured nonwoven material having apertures in portions thereof;

FIG. 29 is a photograph of a two layer apertured nonwoven material having apertures in portions thereof;

FIG. 30 is a photograph of a two layer apertured nonwoven material having apertures in portions thereof;

FIGS. 31-33 are example cross-sectional views of single layer discrete integral second regions with apertures;

FIGS. 34-36 are example cross-sectional views of dual layer discrete integral second regions with apertures in a bottom layer thereof;

FIGS. 37-39 are example cross-sectional views of dual layer discrete integral second regions with apertures in a top layer thereof;

FIGS. 40-42 are example cross-sectional views of dual layer discrete integral second regions with apertures through both layers thereof;

FIG. 43 is an example cross-sectional view of a portion of a three-dimensional material with apertures;

FIG. 44 is an example cross-sectional view of a portion of a three-dimensional material with apertures;

FIG. 45 is a photograph of a nonwoven material having apertures formed by a pin aperturing process;

FIG. 46 is photograph of a nonwoven material having apertures formed by an overbonding and ring rolling process;

FIG. 47 is a schematic representation of an example method for producing the patterned apertured webs of the present disclosure in accordance with the present disclosure;

FIG. 48 is a perspective view of a web weakening arrangement of FIG. 47 in accordance with the present disclosure;

FIG. 49 is a perspective view of an incremental stretching system of the method of FIG. 47 in accordance with the present disclosure;

FIG. 50 is an enlarged view showing the details of teeth of the incremental stretching system of FIG. 49 in accordance with the present disclosure;

FIG. 51 is a perspective view of an example cross machine directional tensioning apparatus of the method of FIG. 47 in accordance with the present disclosure;

FIG. 52 is a schematic representation of a front view of an example cross machine directional tensioning apparatus with outer longitudinal portions in an unexpanded and non-angled position relative to a middle portion in accordance with the present disclosure;

FIG. 53 is a schematic representation of a front view of the cross machine directional tensioning apparatus of FIG. 52 with the outer longitudinal portions in a longitudinally expanded position relative to the middle portion in accordance with the present disclosure;

FIG. 54 is a schematic representation of a front view of the cross machine directional tensioning apparatus of FIG. 52 with the outer longitudinal portions in an angled and expanded position relative to the middle portion in accordance with the present disclosure;

FIG. 55 is a schematic representation of a front view of a cross machine directional tensioning apparatus with outer longitudinal portions fixed in an angled position relative to a middle portion in accordance with the present disclosure;

FIG. 56 is a photograph of a plurality of male forming elements on a roll for use as the male forming member 102 of FIG. 21;

FIG. 57 is an example cross-sectional view of a male forming element;

FIG. 58 is an example cross-sectional view of a female forming element compatible with the male forming element of FIG. 57;

FIG. 59 is an example cross-sectional view of a female forming element compatible with the male forming element of FIG. 57;

FIG. 60 is an example cross-sectional view of a female forming element having a pin;

FIG. 61 is an example cross-sectional view of a male forming element compatible with the female forming element of FIG. 60;

FIG. 62 is a plan view of an example topsheet and acquisition layer configuration for an absorbent article, wearer-facing surface facing the viewer;

FIGS. 63-65 are example cross-sectional views taken about line A-A of FIG. 63;

FIG. 66 is a plan view of another example topsheet and acquisition layer configuration for an absorbent article, wearer-facing surface facing the viewer;

FIG. 67 is an example of a portion of a nested laminate of a topsheet and acquisition layer, with an aperture formed in a distal end of the three-dimensional structure in the topsheet;

FIG. 68 is an example of a portion of a nested laminate of a topsheet and acquisition layer, with an aperture formed in a distal end of the three-dimensional structure in the topsheet;

FIG. 69 is an example of a portion of a nested laminate of a topsheet and acquisition layer with an aperture formed in a distal end of the three-dimensional structure in the topsheet;

FIG. 70 is a photograph of a nested laminate with a pre-apertured top layer (facing viewer) and a non-apertured second layer (under top layer).

### DETAILED DESCRIPTION

Various non-limiting forms of the present disclosure will now be described to provide an overall understanding of the principles of the structure, function, manufacture, and use of the three-dimensional materials having apertures disclosed herein. One or more examples of these non-limiting forms are illustrated in the accompanying drawings. Those of ordinary skill in the art will understand that the three-dimensional materials having apertures described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### I. Definitions

The term “absorbent article” includes disposable articles such as sanitary napkins, panty liners, tampons, interlabial devices, wound dressings, pants, diapers, adult incontinence articles, wipes, and the like. At least some of such absorbent articles are intended for the absorption of body liquids, such as menses or blood, vaginal discharges, urine, and feces. Wipes may be used to absorb body liquids, or may be used for other purposes, such as for cleaning surfaces. Various absorbent articles described above will typically comprise a liquid pervious topsheet, a liquid impervious backsheet joined to the topsheet, and an absorbent core between the topsheet and backsheet. The nonwoven material described herein can comprise at least part of other articles such as scouring pads, wet or dry-mop pads (such as SWIFFER® pads), and the like.

The term “absorbent core”, as used herein, refers to the component of the absorbent article that is primarily responsible for storing liquids. As such, the absorbent core typically does not include the topsheet or backsheet of the absorbent article.

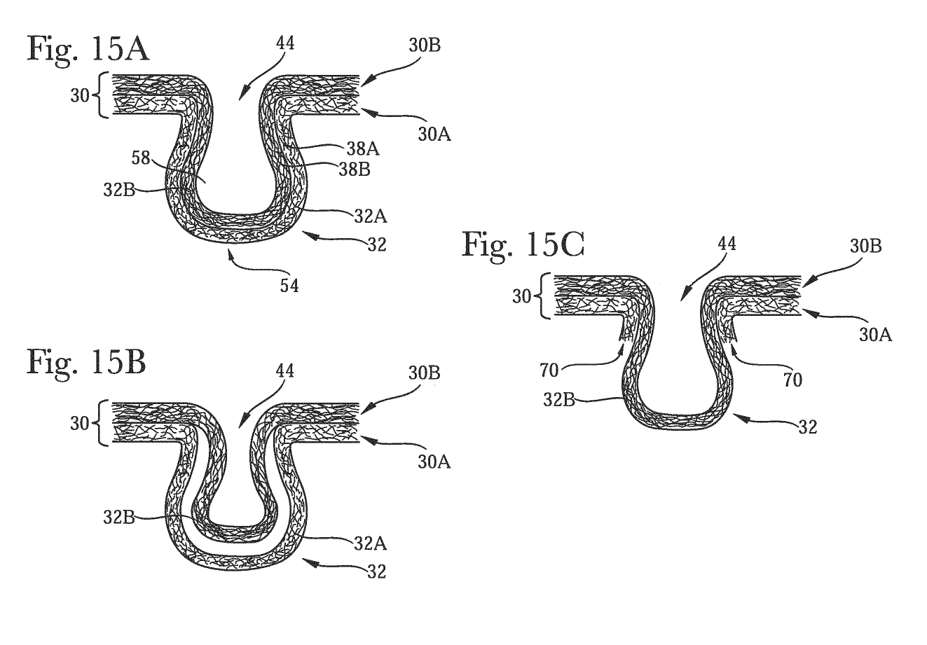

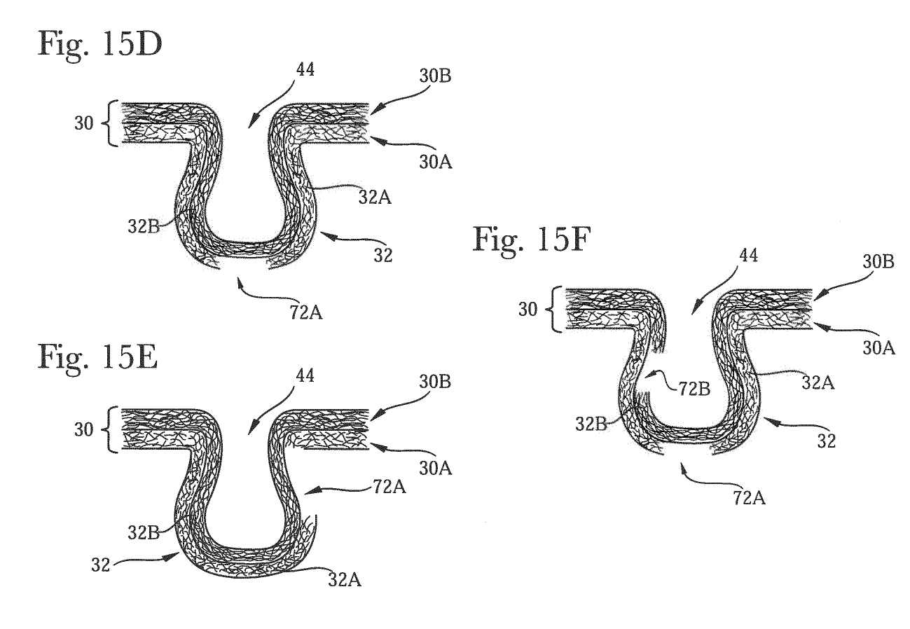

The term “aperture”, as used herein, refers to a predetermined and intentional hole that extends completely through a web or structure (that is, a through hole). The apertures can either be punched cleanly through the web so that the material surrounding the aperture lies in the same plane as the web prior to the formation of the aperture (a “two dimensional” aperture), or the holes can be formed such that at least some of the material surrounding the opening is pushed out of the plane of the web. In the latter case, the apertures may resemble a depression with an aperture therein, and may be referred to herein as a “three dimensional” aperture, a subset of apertures. The term “aperture” does not refer to unintentional variances in the nonwoven material or unintentional tears formed during manufacturing, such as the unintentional tears illustrated in FIGS. 15C-15F, for example.

Characteristic dimensions of the apertures (that is: length, width, aspect ratio, area) are all measured without strain applied at the time of making the measurement using a microscope at 60× magnification. The aspect ratio is defined as ratio between the largest length and the largest width.

The term “component” of an absorbent article, as used herein, refers to an individual constituent of an absorbent article, such as a topsheet, acquisition layer, liquid handling layer, absorbent core or layers of absorbent cores, backsheets, and barriers such as barrier layers and barrier cuffs.

The term “cross-machine direction” or “CD” means the path that is perpendicular to the machine direction in the plane of the web.

The term “deformable material”, as used herein, is a material which is capable of changing its shape or density in response to applied stresses or strains.

The term “discrete”, as used herein, means distinct or unconnected. When the term “discrete” is used relative to forming elements on a forming member, it is meant that the distal (or radially outwardmost) ends of the forming elements are distinct or unconnected in all directions, including in the machine and cross-machine directions (even though bases of the forming elements may be formed into the same surface of a roll, for example).

The term “disposable” is used herein to describe absorbent articles and other products which are not intended to be laundered or otherwise restored or reused as an absorbent article or product (i.e., they are intended to be discarded after use and, preferably, to be recycled, composted or otherwise disposed of in an environmentally compatible manner).

The term “forming elements”, as used herein, refers to any elements on the surface of a forming member that are capable of deforming a web.

The term “integral”, as used herein as in “integral extension” when used to describe the protrusions, refers to fibers of the protrusions having originated from the fibers of the precursor web(s). Thus, as used herein, “integral” is to be distinguished from fibers introduced to or added to a separate precursor web for the purpose of making the protrusions.

The term “joined to” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element. The term “joined to” encompasses configurations in which an element is secured to another element at selected locations, as well as configurations in which an element is completely secured to another element across the entire surface of one of the elements. The term “joined to” includes any known manner in which elements can be secured including, but not limited to mechanical entanglement.

The term “machine direction” or “MD” means the path that material, such as a web, follows through a manufacturing process.

The term “macroscopic”, as used herein, refers to structural features or elements that are readily visible and distinctly discernable to a human having 20/20 vision when the perpendicular distance between the viewer's eye and the web is about 12 inches (30 cm). Conversely, the term “microscopic” refers to such features that are not readily visible and distinctly discernable under such conditions.

The term “mechanically deforming”, as used herein, refers to processes in which a mechanical force is exerted upon a material in order to permanently deform the material.

The term “permanently deformed”, as used herein, refers to the state of a deformable material whose shape or density has been permanently altered in response to applied stresses or strains.

The terms “SELF” and “SELF'ing”, refer to Procter & Gamble technology in which SELF stands for Structural Elastic Like Film. While the process was originally developed for deforming polymer film to have beneficial structural characteristics, it has been found that the SELF'ing process can be used to produce beneficial structures in other materials. Processes, apparatuses, and patterns produced via SELF are illustrated and described in U.S. Pat. Nos. 5,518,801; 5,691,035; 5,723,087; 5,891,544; 5,916,663; 6,027,483; and 7,527,615 B2.

The term “tuft”, as used herein, refers to a particular type of feature that may be formed from fibers in a nonwoven web. Tufts may have a tunnel-like configuration which may be open at both of their ends.

The term “web” is used herein to refer to a material whose primary dimension is X-Y, i.e., along its length (or longitudinal direction) and width (or transverse direction). It should be understood that the term “web” is not necessarily limited to single layers or sheets of material. Thus the web can comprise laminates or combinations of several sheets of the requisite type of materials.

The term “Z-dimension” refers to the dimension orthogonal to the length and width of the web or article. The Z-dimension usually corresponds to the thickness of the web or material. As used herein, the term “X-Y dimension” refers to the plane orthogonal to the thickness of the web or material. The X-Y dimension usually corresponds to the length and width, respectively, of the web or material.

### II. Nonwoven Materials

The present disclosure is directed to nonwoven materials having discrete three-dimensional deformations, which deformations provide protrusions on one side of the material, and openings on the other side of the nonwoven materials. Methods of making the nonwoven materials are also disclosed. The nonwoven materials can be used in absorbent articles and other articles.

As used herein, the term “nonwoven” refers to a web or material having a structure of individual fibers or threads which are interlaid, but not in a repeating pattern as in a woven or knitted fabric, which latter types of fabrics do not typically have randomly oriented or substantially randomly-oriented fibers. Nonwoven webs will have a machine direction (MD) and a cross machine direction (CD) as is commonly known in the art of web manufacture. By “substantially randomly oriented” is meant that, due to processing conditions of the precursor web, there may be a higher amount of fibers oriented in the MD than the CD, or vice versa. For example, in spunbonding and meltblowing processes continuous strands of fibers are deposited on a support moving in the MD. Despite attempts to make the orientation of the fibers of the spunbond or meltblown nonwoven web truly “random,” usually a slightly higher percentage of fibers are oriented in the MD as opposed to the CD.

Nonwoven webs and materials are often incorporated into products, such as absorbent articles, at high manufacturing line speeds. Such manufacturing processes can apply compressive and shear forces on the nonwoven webs that may damage certain types of three-dimensional features that have been purposefully formed in such webs. In addition, in the event that the nonwoven material is incorporated into a product (such as a disposable diaper) that is made or packaged under compression, it becomes difficult to preserve the three-dimensional character of some types of prior three-dimensional features after the material is subjected to such compressive forces.

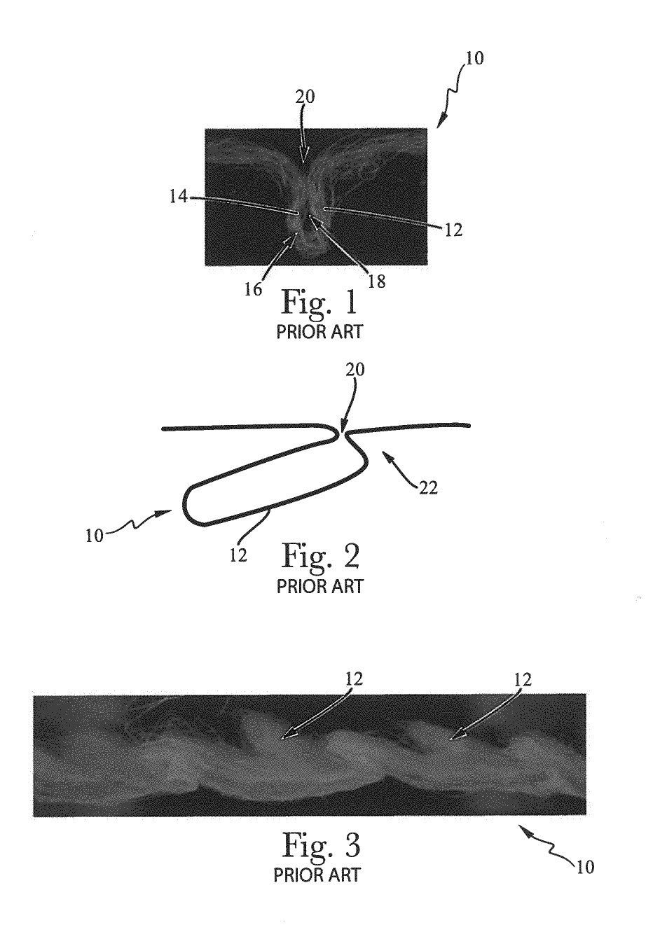

For instance, FIGS. 1 and 2 show an example of a prior art nonwoven material 10 with a tufted structure. The nonwoven material comprises tufts 12 formed from looped fibers 14 that form a tunnel-like structure having two ends 16. The tufts 12 extend outward from the plane of the nonwoven material in the Z-direction. The tunnel-like structure has a width that is substantially the same from one end of the tuft to the opposing end. Often, such tufted structures will have holes or openings 18 at both ends and an opening 20 at their base. Typically, the openings 18 at the ends of the tufts are at the machine direction (MD) ends of the tufts. The openings 18 at the ends of the tufts can be a result of the process used to form the tufts. If the tufts 12 are formed by forming elements in the form of teeth with a relatively small tip and vertical leading and trailing edges that form a sharp point, these leading and/or trailing edges may punch through the nonwoven web at least one of the ends of the tufts. As a result, openings 18 may be formed at one or both ends of the tufts 12.

While such a nonwoven material 10 provides well-defined tufts 12, the opening 20 at the base of the tuft structure can be relatively narrow and difficult to see with the naked eye. In addition, as shown in FIG. 2, the material of the tuft 12 surrounding this narrow base opening 20 may tend to form a hinge 22, or pivot point if forces are exerted on the tuft. If the nonwoven is compressed (such as in the Z-direction), in many cases, the tufts 12 can collapse to one side and close off the opening 20. Typically, a majority of the tufts in such a tufted material will collapse and close off the openings 20. FIG. 2 schematically shows an example of a tuft 12 after it has collapsed. In FIG. 2, the tuft 12 has folded over to the left side. FIG. 3 is an image showing a nonwoven material with several upwardly-oriented tufts, all of which have folded over to the side. However, not all of the tufts 12 will collapse and fold over to the same side. Often, some tufts 12 will fold to one side, and some tufts will fold to the other side. As a result of the collapse of the tufts 12, the openings 20 at the base of the tufts can close up, become slit-like, and virtually disappear.

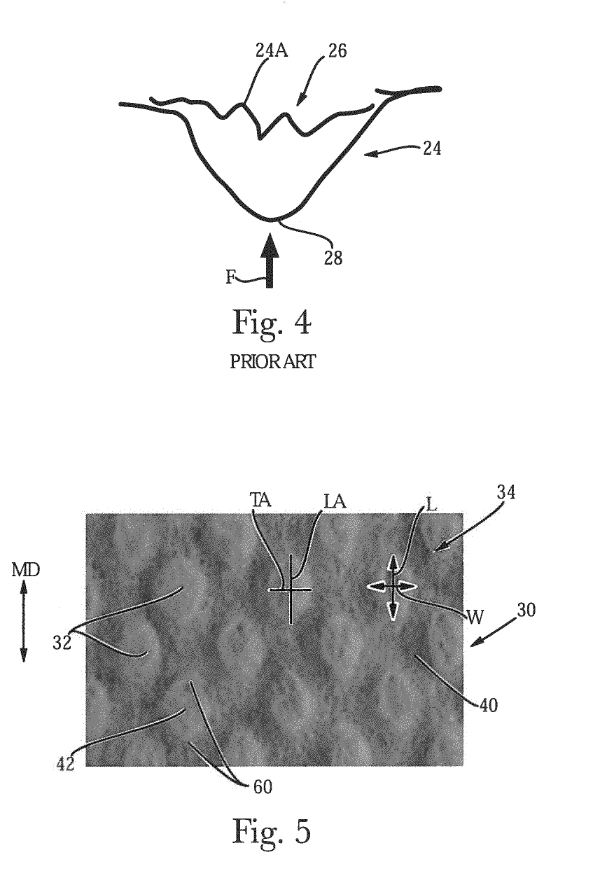

Prior art nonwoven materials with certain other types of three dimensional deformations, such as conical structures, can also be subject to collapse when compressed. As shown in FIG. 4, conical structures 24 will not necessarily fold over as will certain tufted structures when subjected to compressive forces F. However, conical structures 24 can be subject to collapse in that their relatively wide base opening 26 and smaller tip 28 causes the conical structure to push back toward the plane of the nonwoven material, such as to the configuration designated 24A.

The nonwoven materials of at least some embodiments of the present disclosure described herein are intended to better preserve the structure of discrete three-dimensional features in the nonwoven materials after compression.

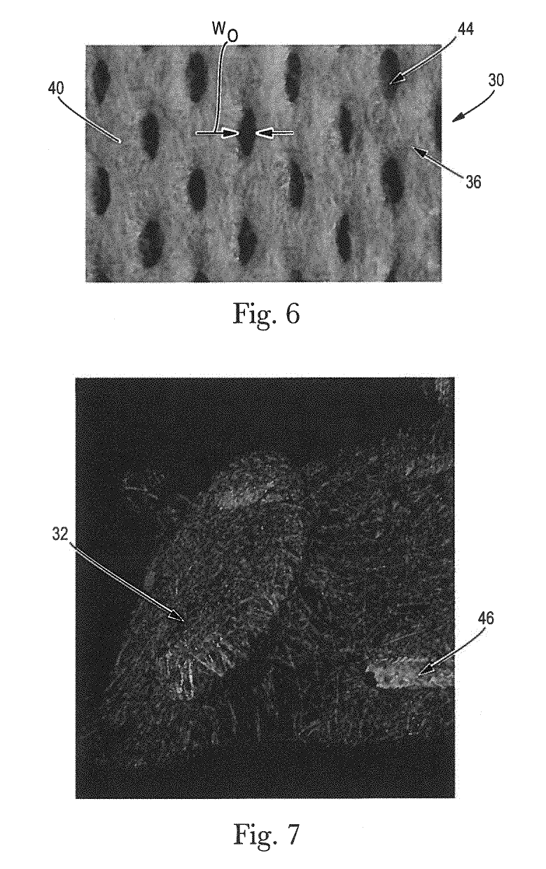

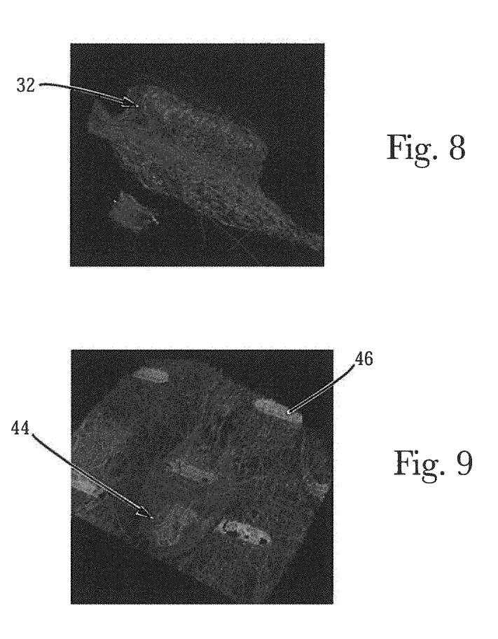

FIGS. 5-14 show examples of nonwoven materials 30 with three-dimensional deformations comprising protrusions 32 therein. The nonwoven materials 30 have a first surface 34, a second surface 36, and a thickness T therebetween (the thickness being shown in FIG. 12). FIG. 5 shows the first surface 34 of a nonwoven material 30 with the protrusions 32 that extend outward from the first surface 34 of the nonwoven material oriented upward. FIG. 6 shows the second surface 36 of a nonwoven material 30 such as that shown in FIG. 5, having three-dimensional deformations formed therein, with the protrusions oriented downward and the base openings 44 oriented upward. FIG. 7 is a Micro CT scan image showing a perspective view of a protrusion 32. FIG. 8 is a Micro CT scan image showing a side view of a protrusion 32 (of one of the longer sides of the protrusion). FIG. 9 is a Micro CT scan image showing a perspective view of a deformation with the opening 44 facing upward. The nonwoven materials 30 comprise a plurality of fibers 38 (shown in FIGS. 7-11 and 14). As shown in FIGS. 7 and 9, in some cases, the nonwoven material 30 may have a plurality of bonds 46 (such as thermal point bonds) therein to hold the fibers 38 together. Any such bonds 46 are typically present in the precursor material from which the nonwoven materials 30 are formed.

The protrusions 32 may, in some cases, be formed from looped fibers (which may be continuous) 38 that are pushed outward so that they extend out of the plane of the nonwoven web in the Z-direction. The protrusions 32 will typically comprise more than one looped fiber. In some cases, the protrusions 32 may be formed from looped fibers and at least some broken fibers. In addition, in the case of some types of nonwoven materials (such as carded materials, which are comprised of shorter fibers), the protrusions 32 may be formed from loops comprising multiple discontinuous fibers. Multiple discontinuous fibers in the form of a loop are shown as layer 30A in FIGS. 15A-15F. The looped fibers may be: aligned (that is, oriented in substantially the same direction); not be aligned; or, the fibers may be aligned in some locations within the protrusions 32, and not aligned in other parts of the protrusions.

In some cases, if male/female forming elements are used to form the protrusions 32, and the female forming elements substantially surround the male forming elements, the fibers in at least part of the protrusions 32 may remain substantially randomly oriented (rather than aligned), similar to their orientation in the precursor web(s). For example, in some cases, the fibers may remain substantially randomly oriented in the cap of the protrusions, but be more aligned in the side walls such that the fibers extend in the Z-direction from the base of the protrusions to the cap. In addition, if the precursor web comprises a multi-layer nonwoven material, the alignment of fibers can vary between layers, and can also vary between different portions of a given protrusion 32 within the same layer.

The nonwoven material 30 may comprise a generally planar first region 40 and the three-dimensional deformations may comprise a plurality of discrete integral second regions 42. The term “generally planar” is not meant to imply any particular flatness, smoothness, or dimensionality. Thus, the first region 40 can include other features that provide the first region 40 with a topography. Such other features can include, but are not limited to small projections, raised network regions around the base openings 44, and other types of features. Thus, the first region 40 is generally planar when considered relative to the second regions 42. The first region 40 can have any suitable plan view configuration. In some cases, the first region 40 is in the form of a continuous inter-connected network which comprises portions that surround each of the deformations.

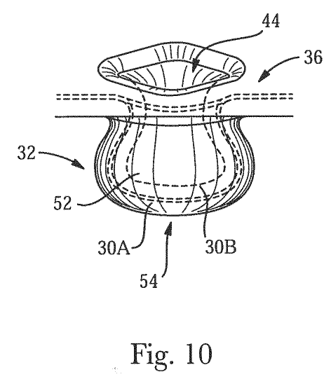

The term “deformation”, as used herein, includes both the protrusions 32 formed on one side of the nonwoven material and the base openings 44 formed in the opposing side of the material. The base openings 44 are most often not in the form of an aperture or a through-hole. The base openings 44 may instead appear as depressions. The base openings 44 can be analogized to the opening of a bag. A bag has an opening that typically does not pass completely through the bag. In the case of the present nonwoven materials 30, as shown in FIG. 10, the base openings 44 open into the interior of the protrusions 32.

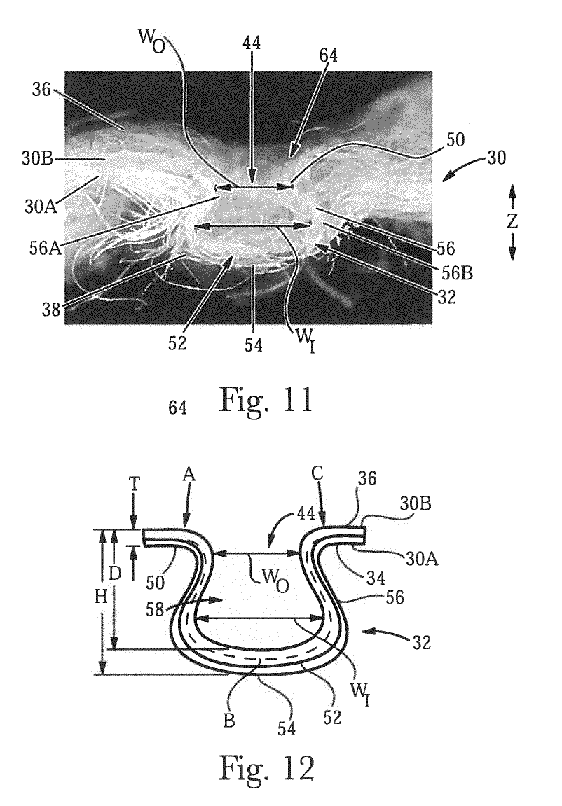

FIG. 11 shows one example of a multi-layer nonwoven material 30 having a three-dimensional deformation in the form of a protrusion 32 on one side of the material that provides a wide base opening 44 on the other side of the material. The dimensions of “wide” base openings are described in further detail below. In this case, the base opening 44 is oriented upward in the figure. When there is more than one nonwoven layer, the individual layers can be designated 30A, 30B, etc. The individual layers 30A and 30B each have first and second surfaces, which can be designated similarly to the first and second surfaces 34 and 36 of the nonwoven material (e.g., 34A and 36A for the first and second surfaces of the first layer 30A; and, 34B and 36B for the first and second surfaces of the second layer 30B).

As shown in FIGS. 11 and 12, the protrusions 32 comprise: a base 50 proximate the first surface 34 of the nonwoven material; an opposed enlarged distal portion or cap portion, or “cap” 52, that extends to a distal end 54; side walls (or “sides”) 56; an interior 58; and a pair of ends 60 (the latter being shown in FIG. 5). The “base” 50 of the protrusions 32 comprises the narrowest portion of the protrusion when viewed from one of the ends of the protrusion. The term “cap” does not imply any particular shape, other than it comprises the wider portion of the protrusion 32 that includes and is adjacent to the distal end 54 of the protrusion 32. The side walls 56 have an inside surface 56A and an outside surface 56B. As shown in FIGS. 11 and 12, the side walls 56 transition into, and may comprise part of the cap 52. Therefore, it is not necessary to precisely define where the side walls 56 end and the cap 52 begins. The cap 52 will have a maximum interior width, W_{I}, between the inside surfaces 56A of the opposing side walls 56. The cap 52 will also have a maximum exterior width W between the outside surfaces 56B of the opposing side walls 56. The ends 60 of the protrusions 32 are the portions of the protrusions that are spaced furthest apart along the longitudinal axis, L, of the protrusions.

As shown in FIGS. 11 and 12, the narrowest portion of the protrusion 32 defines the base opening 44. The base opening 44 has a width W_{O}. The base opening 44 may be located (in the z-direction) between the plane defined by the second surface 36 of the material and the distal end 54 of the protrusion. As shown in FIGS. 11 and 12, the nonwoven material 30 may have an opening in the second surface 36 (the “second surface opening” 64) that transitions into the base opening 44 (and vice versa), and is the same size as, or larger than the base opening 44. The base opening 44 will, however, generally be discussed more frequently herein since its size will often be more visually apparent to the consumer in those embodiments where the nonwoven material 30 is placed in an article with the base openings 44 visible to the consumer. It should be understood that in certain embodiments, such as in some embodiments in which the base openings 44 face outward (for example, toward a consumer and away from the absorbent core in an absorbent article), it may be desirable for the base openings 44 not to be covered and/or closed off by another web.

As shown in FIG. 12, the protrusions 32 have a depth D measured from the second surface 36 of the nonwoven web to the interior of the protrusion at the distal end 54 of the protrusions. The protrusions 32 have a height H measured from the second surface 36 of the nonwoven web to the distal end 54 of the protrusions. In most cases the height H of the protrusions 32 will be greater than the thickness T of the first region 40. The relationship between the various portions of the deformations may be such that as shown in FIG. 11, when viewed from the end, the maximum interior width W_{I} of the cap 52 of the protrusions is wider than the width, W_{O}, of the base opening 44.

The protrusions 32 may be of any suitable shape. Since the protrusions 32 are three-dimensional, describing their shape depends on the angle from which they are viewed. When viewed from above (that is, perpendicular to the plane of the web, or plan view) such as in FIG. 5, suitable shapes include, but are not limited to: circular, diamond-shaped, rounded diamond-shaped, U.S. football-shaped, oval-shaped, clover-shaped, heart-shaped, triangle-shaped, tear-drop shaped, and elliptical-shaped. (The base openings 44 will typically have a shape similar to the plan view shape of the protrusions 32.) In other cases, the protrusions 32 (and base openings 44) may be non-circular. The protrusions 32 may have similar plan view dimensions in all directions, or the protrusions may be longer in one dimension than another. That is, the protrusions 32 may have different length and width dimensions. If the protrusions 32 have a different length than width, the longer dimension will be referred to as the length of the protrusions. The protrusions 32 may, thus, have a ratio of length to width, or an aspect ratio. The aspect ratios can range from about 1:1 to about 10:1.

As shown in FIG. 5, the protrusions 32 may have a width, W, that varies from one end 60 to the opposing end 60 when the protrusions are viewed in plan view. The width W may vary with the widest portion of the protrusions in the middle of the protrusions, and the width of the protrusions decreasing at the ends 60 of the protrusions. In other cases, the protrusions 32 could be wider at one or both ends 60 than in the middle of the protrusions. In still other cases, protrusions 32 can be formed that have substantially the same width from one end of the protrusion to the other end of the protrusion. If the width of the protrusions 32 varies along the length of the protrusions, the portion of the protrusion where the width is the greatest is used in determining the aspect ratio of the protrusions.

When the protrusions 32 have a length L that is greater than their width W, the length of the protrusions may be oriented in any suitable direction relative to the nonwoven material 30. For example, the length of the protrusions 32 (that is, the longitudinal axis, LA, of the protrusions) may be oriented in the machine direction, the cross-machine direction, or any desired orientation between the machine direction and the cross-machine direction. The protrusions 32 also have a transverse axis TA generally orthogonal to the longitudinal axis LA in the MD-CD plane. In the embodiment shown in FIGS. 5 and 6, the longitudinal axis LA is parallel to the MD. In some embodiments, all the spaced apart protrusions 32 may have generally parallel longitudinal axes LA.

The protrusions 32 may have any suitable shape when viewed from the side. Suitable shapes include those in which there is a distal portion or “cap” with an enlarged dimension and a narrower portion at the base when viewed from at least one side. The term “cap” is analogous to the cap portion of a mushroom. (The cap does not need to resemble that of any particular type of mushroom. In addition, the protrusions 32 may, but need not, have a mushroom-like stem portion.) In some cases, the protrusions 32 may be referred to as having a bulbous shape when viewed from the end 60, such as in FIG. 11. The term “bulbous”, as used herein, is intended to refer to the configuration of the protrusions 32 as having a cap 52 with an enlarged dimension and a narrower portion at the base when viewed from at least one side (particularly when viewing from one of the shorter ends 60) of the protrusion 32. The term “bulbous” is not limited to protrusions that have a circular or round plan view configuration that is joined to a columnar portion. The bulbous shape, in the embodiment shown (where the longitudinal axis LA of the deformations 32 is oriented in the machine direction), may be most apparent if a section is taken along the transverse axis TA of the deformation (that is, in the cross-machine direction). The bulbous shape may be less apparent if the deformation is viewed along the length (or longitudinal axis LA) of the deformation such as in FIG. 8.

The protrusions 32 may comprise fibers 38 that at least substantially surround the sides of the protrusions. This means that there are multiple fibers that extend (e.g., in the Z-direction) from the base 50 of the protrusions 32 to the distal end 54 of the protrusions, and contribute to form a portion of the sides 56 and cap 52 of a protrusion. In some cases, the fibers may be substantially aligned with each other in the Z-direction in the sides 56 of the protrusions 32. The phrase “substantially surround”, thus, does not require that each individual fiber be wrapped in the X-Y plane substantially or completely around the sides of the protrusions. If the fibers 38 are located completely around the sides of the protrusions, this would mean that the fibers are located 360° around the protrusions. The protrusions 32 may be free of large openings at their ends 60, such as those openings 18 at the leading end and trailing end of the tufts shown in FIG. 1. In some cases, the protrusions 32 may have an opening at only one of their ends, such as at their trailing end. The protrusions 32 also differ from embossed structures such as shown in FIG. 4. Embossed structures typically do not have distal portions that are spaced perpendicularly away (that is, in the Z-direction) from their base that are wider than portions that are adjacent to their base, as in the case of the cap 52 on the present protrusions 32.

The protrusions 32 may have certain additional characteristics. As shown in FIGS. 11 and 12, the protrusions 32 may be substantially hollow. As used herein, the term “substantially hollow” refers to structures which the protrusions 32 are substantially free of fibers in interior of protrusions. The term “substantially hollow”, does not, however, require that the interior of the protrusions must be completely free of fibers. Thus, there can be some fibers inside the protrusions. “Substantially hollow” protrusions are distinguishable from filled three-dimensional structures, such as those made by laying down fibers, such as by airlaying or carding fibers onto a forming structure with recesses therein.

The side walls 56 of the protrusions 32 can have any suitable configuration. The configuration of the side walls 56, when viewed from the end of the protrusion such as in FIG. 11, can be linear or curvilinear, or the side walls can be formed by a combination of linear and curvilinear portions. The curvilinear portions can be concave, convex, or combinations of both. For example, the side walls 56 in the embodiment shown in FIG. 11 comprise portions that are curvilinear concave inwardly near the base of the protrusions and convex outwardly near the cap of the protrusions. The sidewalls 56 and the area around the base opening 44 of the protrusions may, under 20× magnification, have a visibly significantly lower concentration of fibers per given area (which may be evidence of a lower basis weight or lower opacity) than the portions of the nonwoven in the unformed first region 40. The protrusions 32 may also have thinned fibers in the sidewalls 56. The fiber thinning, if present, will be apparent in the form of necked regions in the fibers 38 as seen in scanning electron microscope (SEM) images taken at 200× magnification. Thus, the fibers may have a first cross-sectional area when they are in the undeformed nonwoven precursor web, and a second cross-sectional area in the side walls 56 of the protrusions 32 of the deformed nonwoven web, wherein the first cross-sectional area is greater than the second cross-sectional area. The side walls 56 may also comprise some broken fibers as well. In some embodiments, the side walls 56 may comprise greater than or equal to about 30%, alternatively greater than or equal to about 50% broken fibers.

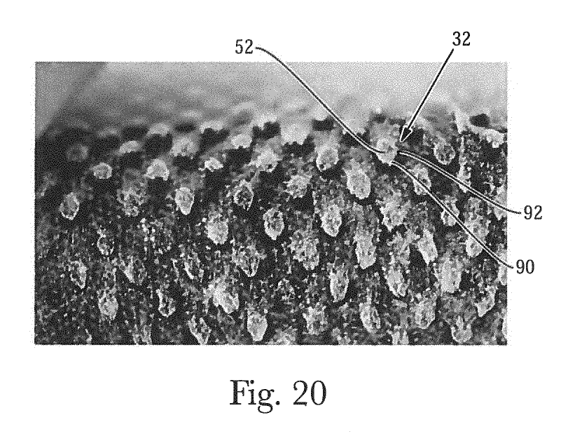

In some embodiments, the distal end 54 of the protrusions 32 may be comprised of original basis weight, non-thinned, and non-broken fibers. If the base opening 44 faces upward, the distal end 54 will be at the bottom of the depression that is formed by the protrusion. The distal end 54 will be free from apertures formed completely through the distal end. Thus, the nonwoven materials may be nonapertured. The term “apertures”, as used herein, refers to holes formed in the nonwovens after the formation of the nonwovens, and does not include the pores typically present in nonwovens. The term “apertures” also does not refer to irregular breaks (or interruptions) in the nonwoven material(s) such as shown in FIGS. 15D-15F and FIG. 20 resulting from localized tearing of the material(s) during the process of forming deformations therein, which breaks may be due to variability in the precursor material(s). The distal end 54 may have relatively greater fiber concentration in comparison to the remaining portions of the structure that forms the protrusions. The fiber concentration can be measured by viewing the sample under a microscope and counting the number of fibers within an area. As described in greater detail below, however, if the nonwoven web is comprised of more than one layer, the concentration of fibers in the different portions of the protrusions may vary between the different layers.

The protrusions 32 may be of any suitable size. The size of the protrusions 32 can be described in terms of protrusion length, width, caliper, height, depth, cap size, and opening size. (Unless otherwise stated, the length L and width W of the protrusions are the exterior length and width of the cap 52 of the protrusions.) The dimensions of the protrusions and openings can be measured before and after compression (under either a pressure of 7 kPa or 35 KPa, whichever is specified) in accordance with the Accelerated Compression Method described in the Test Methods section. The protrusions have a caliper that is measured between the same points as the height H, but under a 2 KPa load, in accordance with the Accelerated Compression Method. All dimensions of the protrusions and openings other than caliper (that is, length, width, height, depth, cap size, and opening size) are measured without pressure applied at the time of making the measurement using a microscope at 20× magnification.

In some embodiments, the length of the cap 52 may be in a range from about 1.5 mm to about 10 mm. In some embodiments, the width of the cap (measured where the width is the greatest) may be in a range from about 1.5 mm to about 5 mm. The cap portion of the protrusions may have a plan view surface area of at least about 3 mm². In some embodiments, the protrusions may have a pre-compression height H that is in a range from about 1 mm to about 10 mm, alternatively from about 1 mm to about 6 mm. In some embodiments, the protrusions may have a post-compression height H that is in a range from about 0.5 mm to about 6 mm, alternatively from about 0.5 mm to about 1.5 mm. In some embodiments, the protrusions may have a depth D, in an uncompressed state that is in a range from about 0.5 mm to about 9 mm, alternatively from about 0.5 mm to about 5 mm. In some embodiments, the protrusions may have a depth D, after compression that is in a range from about 0.25 mm to about 5 mm, alternatively from about 0.25 mm to about 1 mm.

The nonwoven material 30 can comprise a composite of two or more nonwoven materials that are joined together. In such a case, the fibers and properties of the first layer will be designated accordingly (e.g., the first layer is comprised of a first plurality of fibers), and the fibers and properties of the second and subsequent layers will be designated accordingly (e.g., the second layer is comprised of a second plurality of fibers). In a two or more layer structure, there are a number of possible configurations the layers may take following the formation of the deformations therein. These will often depend on the extensibility of the nonwoven materials used for the layers. It is desirable that at least one of the layers have deformations which form protrusions 32 as described herein in which, along at least one cross-section, the width of the cap 52 of the protrusions is greater than the width of the base opening 44 of the deformations. For example, in a two layer structure where one of the layers will serve as the topsheet of an absorbent article and the other layer will serve as an underlying layer (such as an acquisition layer), the layer that has protrusions therein may comprise the topsheet layer. The layer that most typically has a bulbous shape will be the one which is in contact with the male forming member during the process of deforming the web. FIG. 15A-FIG. 15E show different alternative embodiments of three-dimensional protrusions 32 in multiple layer materials.

In certain embodiments, such as shown in FIGS. 11, 12, and 15A, similar-shaped looped fibers may be formed in each layer of multiple layer nonwoven materials, including in the layer 30A that is spaced furthest from the discrete male forming elements during the process of forming the protrusions 32 therein, and in the layer 30B that is closest to the male forming elements during the process. In the protrusions 32, portions of one layer such as 30B may fit within the other layer, such as 30A. These layers may be referred to as forming a “nested” structure in the protrusions 32. Formation of a nested structure may require the use of two (or more) highly extensible nonwoven precursor webs. In the case of two layer materials, nested structures may form two complete loops, or (as shown in some of the following drawing figures) two incomplete loops of fibers.

As shown in FIG. 15A, a three-dimensional protrusion 32 comprises protrusions 32A formed in the first layer 30A and protrusions 32B formed in the second layer 30B. In one embodiment, the first layer 30A may be incorporated into an absorbent article as an acquisition layer, and the second layer 30B may be a topsheet, and the protrusions formed by the two layers may fit together (that is, are nested). In this embodiment, the protrusions 32A and 32B formed by the first and second layers 30A and 30B fit closely together. The three-dimensional protrusion 32A comprises a plurality of fibers 38A and the three-dimensional protrusion 32B comprises a plurality of fibers 38B. The three-dimensional protrusion 32B is nested into the three-dimensional protrusion 32A. In the embodiment shown, the fibers 38A in the first layer 30A are shorter in length than the fibers 38B in the second layer 30B. In other embodiments, the relative length of fibers in the layers may be the same, or in the opposite relationship wherein the fibers in the first layer are longer than those in the second layer. In addition, in this embodiment, and any of the other embodiments described herein, the nonwoven layers can be inverted when incorporated into an absorbent article, or other article, so that the protrusions 32 face upward (or outward). In such a case, the material suitable for the topsheet will be used in layer 30A, and material suitable for the underlying layer will be used in layer 30B.

FIG. 15B shows that the nonwoven layers need not be in a contacting relationship within the entirety of the protrusion 32. Thus, the protrusions 32A and 32B formed by the first and second layers 30A and 30B may have different heights and/or widths. The two materials may have substantially the same shape in the protrusion 32 as shown in FIG. 15B (where one of the materials has the same the curvature as the other). In other embodiments, however, the layers may have different shapes. It should be understood that FIG. 15B shows only one possible arrangement of layers, and that many other variations are possible, but that as in the case of all the figures, it is not possible to provide a drawing of every possible variation.

As shown in FIG. 15C, one of the layers, such as first layer 30A (e.g., an acquisition layer) may be ruptured in the area of the three-dimensional protrusion 32. As shown in FIG. 15C, the protrusions 32 are only formed in the second layer 30B (e.g., the topsheet) and extend through openings in the first layer 30A. That is, the three-dimensional protrusion 32B in the second layer 30B interpenetrates the ruptured first layer 30A. Such a structure may place the topsheet in direct contact an underlying distribution layer or absorbent core, which may lead to improved dryness. In such an embodiment, the layers are not considered to be “nested” in the area of the protrusion. (In the other embodiments shown in FIGS. 15D-15F, the layers would still be considered to be “nested”.) Such a structure may be formed if the material of the second layer 30B is much more extensible than the material of the first layer 30A. In such a case, the openings can be formed by locally rupturing first precursor web by the process described in detail below. The ruptured layer may have any suitable configuration in the area of the protrusion 32. Rupture may involve a simple splitting open of first precursor web, such that the opening in the first layer 30A remains a simple two-dimensional aperture. However, for some materials, portions of the first layer 30A can be deflected or urged out-of-plane (i.e., out of the plane of the first layer 30A) to form flaps 70. The form and structure of any flaps is highly dependent upon the material properties of the first layer 30A. Flaps can have the general structure shown in FIG. 15C. In other embodiments, the flaps 70 can have a more volcano-like structure, as if the protrusion 32B is erupting from the flaps.

Alternatively, as shown in FIGS. 15D-15F, one or both of the first layer 30A and the second layer 30B may be interrupted (or have a break therein) in the area of the three-dimensional protrusion 32. FIGS. 15D and 15E show that the three-dimensional protrusion 32A of the first layer 30A may have an interruption 72A therein. The three-dimensional protrusion 32B of the non-interrupted second layer 30B may coincide with and fit together with the three-dimensional protrusion 32A of the interrupted first layer 30A. Alternatively, FIG. 15F shows an embodiment in which both the first and second layers 30A and 30B have interruptions, or breaks, therein (72A and 72B, respectively). In this case, the interruptions in the layers 30A and 30B are in different locations in the protrusion 32. FIGS. 15D-15F show unintentional random or inconsistent breaks in the materials typically formed by random fiber breakage, which are generally misaligned and can be in the first or second layer, but are not typically aligned and completely through both layers. Thus, there typically will not be an aperture formed completely through all of the layers at the distal end 54 of the protrusions 32.

For dual layer and other multiple layer structures, the basis weight distribution (or the concentration of fibers) within the deformed material 30, as well as the distribution of any thermal point bonds 46 can be different between the layers. As used herein, the term “fiber concentration” has a similar meaning as basis weight, but fiber concentration refers to the number of fibers/given area, rather than g/area as in basis weight. In the case of bond sites 46, the fibers may be melted which may increase the density of the material in the bond sites 46, but the number of fibers will typically be the same as before melting.

Some such dual and multiple layer nonwoven materials may be described in terms of such differences between layers, without requiring one or more of the other features described herein (such as characteristics of the cap portion; controlled collapse under compression; and varying width of the protrusions). Of course such dual and multiple layer nonwoven materials may have any of these other features.

In such dual and multiple layer nonwoven materials each of the layers comprises a plurality of fibers, and in certain embodiments, the protrusions 32 will be formed from fibers in each of the layers. For example, one of the layers, a first layer, may form the first surface 34 of the nonwoven material 30, and one of the layers, a second layer, may form the second surface 36 of the nonwoven material 30. A portion of the fibers in the first layer form part of: the first region 40, the side walls 56 of the protrusions, and the distal ends 54 of the protrusions 32. A portion of the fibers in the second layer form part of: the first region 40, the side walls 56 of the protrusions, and the distal ends 54 of the protrusions 32.

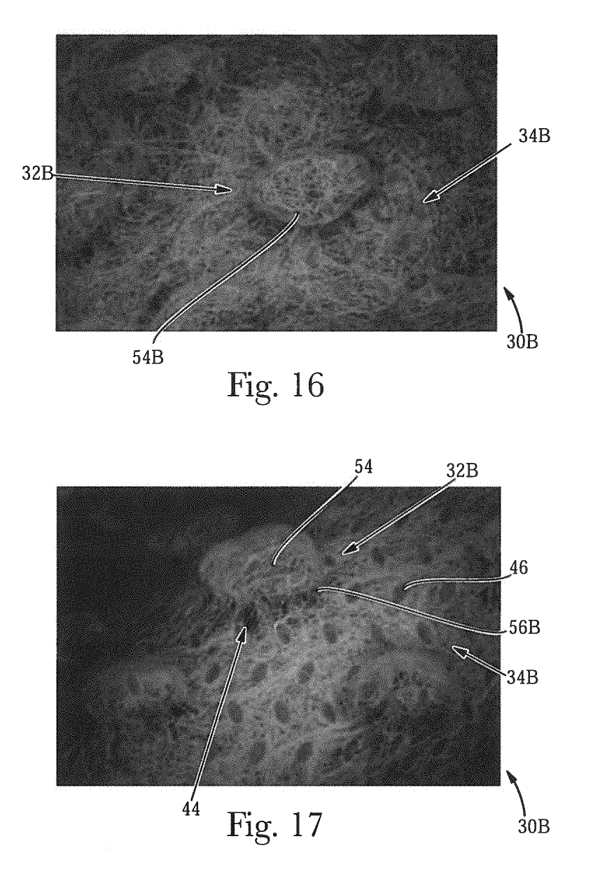

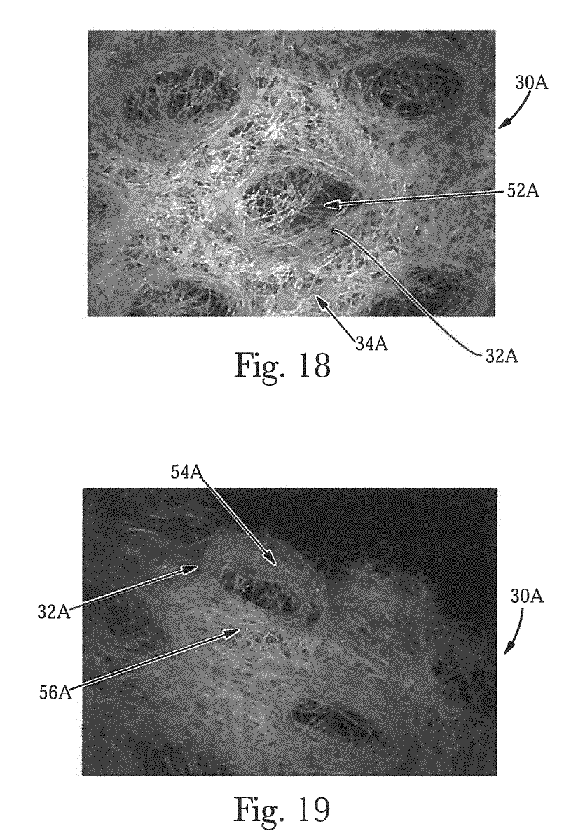

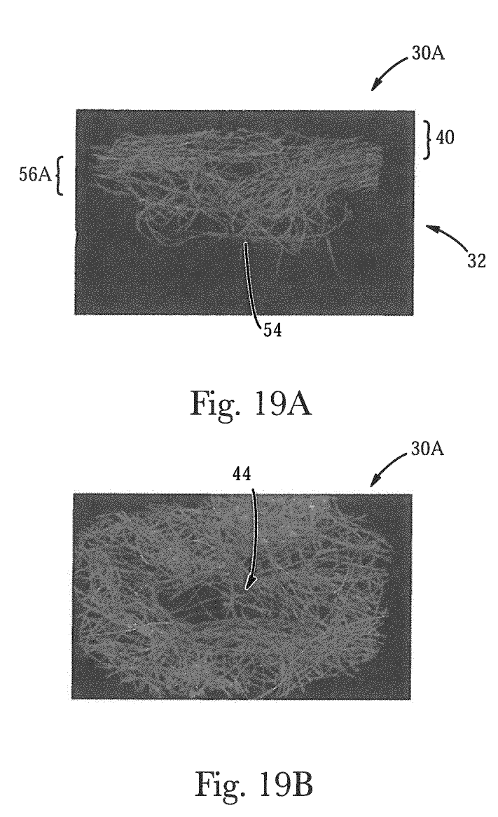

As shown in FIG. 16, the nonwoven layer in contact with the male forming element (e.g., 30B) may have a large portion at the distal end 54B of the protrusion 32B with a similar basis weight to the original nonwoven (that is, to the first region 40). As shown in FIG. 17, the basis weight in the sidewalls 56B of the protrusion 32B and near the base opening 44 may be lower than the basis weight of the first region 40 of the nonwoven layer and the distal end 54 of the protrusion 32B. As shown in FIG. 18, the nonwoven layer in contact with the female forming element (e.g., 30A) may, however, have significantly less basis weight in the cap 52A of the protrusion 32A than in the first region 40 of the nonwoven layer. As shown in FIG. 19, the sidewalls 56A of the protrusion 32A may have less basis weight than the first region 40 of the nonwoven. FIGS. 19A and 19B show that the nonwoven layer 30A in contact with the female forming element may have a fiber concentration that is greatest in the first region 40 (at the upper part of the image in FIG. 19A) and lowest at the distal end 54 of the protrusion 32. The fiber concentration in the side wall 56A, in this case, may be less than that of the first region 40, but greater than that at the distal end 54 of the protrusion 32.

Forming deformations in the nonwoven material may also affect the bonds 46 (thermal point bonds) within the layer (or layers). In some embodiments, the bonds 46 within the distal end 54 of the protrusions 32 may remain intact (not be disrupted) by the deformation process that formed the protrusions 32. In the side walls 56 of the protrusions 32, however, the bonds 46 originally present in the precursor web may be disrupted. When it is said that the bonds 46 may be disrupted, this can take several forms. The bonds 46 can be broken and leave remnants of a bond. In other cases, such as where the nonwoven precursor material is underbonded, the fibers can disentangle from a lightly formed bond site (similar to untying a bow), and the bond site will essentially disappear. In some cases, after the deformation process, the side walls 56 of at least some of the protrusions 32 may be substantially free (or completely free) of thermal point bonds.

Numerous embodiments of dual layer and other multiple layer structures are possible. For example, a nonwoven layer 30B such as that shown in FIGS. 16 and 17 could be oriented with its base openings facing upward, and could serve as a topsheet of a dual or multiple layer nonwoven structure (with at least one other layer serving as an acquisition layer). In this embodiment, the bonds 46 within first region 40 of nonwoven layer 30B and the distal end 54 of the protrusions 32 remain intact. In the side walls 56 of the protrusions 32, however, the bonds 46 originally present in the precursor web are disrupted such that the side walls 56 are substantially free of thermal point bonds. Such a topsheet could be combined with an acquisition layer in which the concentration of fibers within the layer 30A in the first region 40 and the distal end 54 of the protrusions 32 is also greater than the concentration of fibers in the side walls 56 of the protrusions 32.

In other embodiments, the acquisition layer 30A described in the preceding paragraph may have thermal point bonds 46 within first region 40 of nonwoven layer 30B and the distal end 54 of the protrusions 32 that remain intact. In the side walls 56 of the protrusions 32, however, the bonds 46 originally present in the precursor web comprising the acquisition layer 30A are disrupted such that the side walls 56 of the acquisition layer 30A are substantially free of thermal point bonds. In other cases, the thermal point bonds in the acquisition layer 30A at the top of the protrusions 32 may also be disrupted so that the distal end 54 of at least some of the protrusions are substantially or completely free of thermal point bonds.

In other embodiments, a dual layer or multiple layer structure may comprise a topsheet and an acquisition layer that is oriented with its base openings facing upward in which the concentration of fibers at the distal end 54 of each layer (relative to other portions of the layer) differs between layers. For example, in one embodiment, in the layer that forms the topsheet (second layer), the concentration of fibers in the first region and the distal ends of the protrusions are each greater than the concentration of fibers in the side walls of the protrusions. In the layer that forms the acquisition layer (first layer), the concentration of fibers in the first region of the acquisition layer may be greater than the concentration of fibers in the distal ends of the protrusions. In a variation of this embodiment, the concentration of fibers in the first region of the first layer (acquisition layer) is greater than the concentration of fibers in the side walls of the protrusions in the first layer, and the concentration of fibers in the side walls of the protrusions in the first layer is greater than the concentration of fibers forming the distal ends of the protrusions in the first layer. In some embodiments in which the first layer comprises a spunbond nonwoven material (in which the precursor material had thermal point bonds distributed substantially evenly throughout), a portion of the fibers that form the first region in the first layer comprise thermal point bonds, and the portion of the fibers in the first layer forming the side walls and distal ends of at least some of the protrusions may be substantially free of thermal point bonds. In these embodiments, in at least some of the protrusions, at least some of the fibers in the first layer may form a nest or circle around (that is, encircle) the perimeter of the protrusion at the transition between the wide wall and the base of the protrusion as shown in FIG. 19.

The base openings 44 can be of any suitable shape and size. The shape of the base opening 44 will typically be similar to, or the same as, the plan view shape of the corresponding protrusions 32. The base opening 44 may have a width that is greater than about any of the following dimensions before (and after compression): 0.5 mm, 0.7 mm, 0.8 mm, 0.9 mm, 1 mm, or any 0.1 mm increment above 1 mm. The width of the base opening 44 may be in a range that is from any of the foregoing amounts up to about 4 mm, or more. The base openings 44 may have a length that ranges from about 1.5 mm or less to about 10 mm, or more. The base openings 44 may have an aspect ratio that ranges from about 1:1 to 20:1, alternatively from about 1:1 to 10:1. Measurements of the dimensions of the base opening can be made on a photomicrograph. When the size of the width of the base opening 44 is specified herein, it will be appreciated that if the openings are not of uniform width in a particular direction, the width, W_{O}, is measured at the widest portion as shown in FIG. 6. The nonwoven materials of the present disclosure and the method of making the same may create deformations with a wider opening than certain prior structures which have a narrow base. This allows the base openings 44 to be more visible to the naked eye. The width of the base opening 44 is of interest because, being the narrowest portion of the opening, it will be most restrictive of the size of the opening. The deformations retain their wide base openings 44 after compression perpendicular to the plane of the first region 40.

The deformations may compress under load. In some cases, it may be desirable that the load is low enough so that, if the nonwoven is worn against a wearer's body, with the deformations in contact with the wearer's body, the deformations will be soft and will not imprint the skin. This applies in cases where either the protrusions 32 or the base openings 44 are oriented so that they are in contact with the wearer's body. For example, it may be desirable for the deformations to compress under pressures of 2 kPa or less. In other cases, it will not matter if the deformations imprint the wearer's skin. It may be desirable for at least one of the protrusions 32 in the nonwoven material 30 to collapse or buckle in the controlled manner described below under the 7 kPa load when tested in accordance with the Accelerated Compression Method in the Test Methods section below. Alternatively, at least some, or in other cases, a majority of the protrusions 32 may collapse in the controlled manner described herein. Alternatively, substantially all of the protrusions 32 may collapse in the controlled manner described herein. The ability of the protrusions 32 to collapse may also be measured under a load of 35 kPa. The 7 kPa and 35 kPa loads simulate manufacturing and compression packaging conditions. Wear conditions can range from no or limited pressure (if the wearer is not sitting on the absorbent article) up to 2 kPa, 7 kPa, or more.

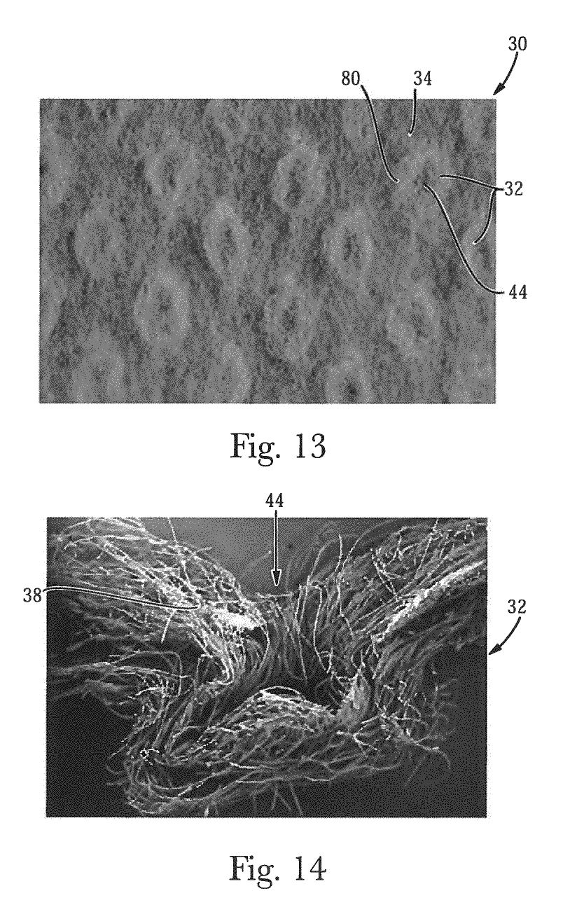

The protrusions 32 may collapse in a controlled manner after compression to maintain the wide opening 44 at the base. FIG. 13 shows the first surface 34 of a nonwoven material 30 according to the present disclosure after it has been subjected to compression. FIG. 14 is a side view of a single downwardly-oriented protrusion 32 after it has been subjected to compression. As shown in FIG. 13, when the protrusions 32 have been compressed, there appears to be a higher concentration of fibers in the form of a ring of increased opacity 80 around the base opening 44. When a compressive force is applied to the nonwoven materials, the side walls 56 of the protrusions 32 may collapse in a more desirable/controlled manner such that the side walls 56 become concave and fold into regions of overlapping layers (such as into an s-shape/accordion-shape). The ring of increased opacity 80 represents folded layers of material. In other words, the protrusions 32 may have a degree of dimensional stability in the X-Y plane when a Z-direction force is applied to the protrusions. It is not necessary that the collapsed configuration of the protrusions 32 be symmetrical, only that the collapsed configuration prevent the protrusions 32 from flopping over or pushing back into the original plane of the nonwoven, and significantly reducing the size of the base opening (for example, by 50% or more). For example, as shown in FIG. 14, the left side of the protrusion 32 can form a z-folded structure, and the right side of the protrusion does not, but still appears, when viewed from above, to have higher opacity due to a degree of overlapping of the material in the folded portion. Without wishing to be bound to any particular theory, it is believed that the wide base opening 44 and large cap 52 (greater than the width of the base opening 44), combined with the lack of a pivot point, causes the protrusions 32 to collapse in a controlled manner (prevents the protrusion 32 from flopping over). Thus, the protrusions 32 are free of a hinge structure that would otherwise permit them to fold to the side when compressed. The large cap 52 also prevents the protrusion 32 from pushing back into the original plane of the nonwoven.

The deformations can be disposed in any suitable density across the surface of the nonwoven material 30. The deformations may, for example, be present in a density of: from about 5 to about 100 deformations; alternatively from about 10 to about 50 deformations; alternatively from about 20 to about 40 deformations, in an area of 10 cm².

The deformations can be disposed in any suitable arrangement across the plane of the nonwoven material. Suitable arrangements include, but are not limited to: staggered arrangements, and zones.

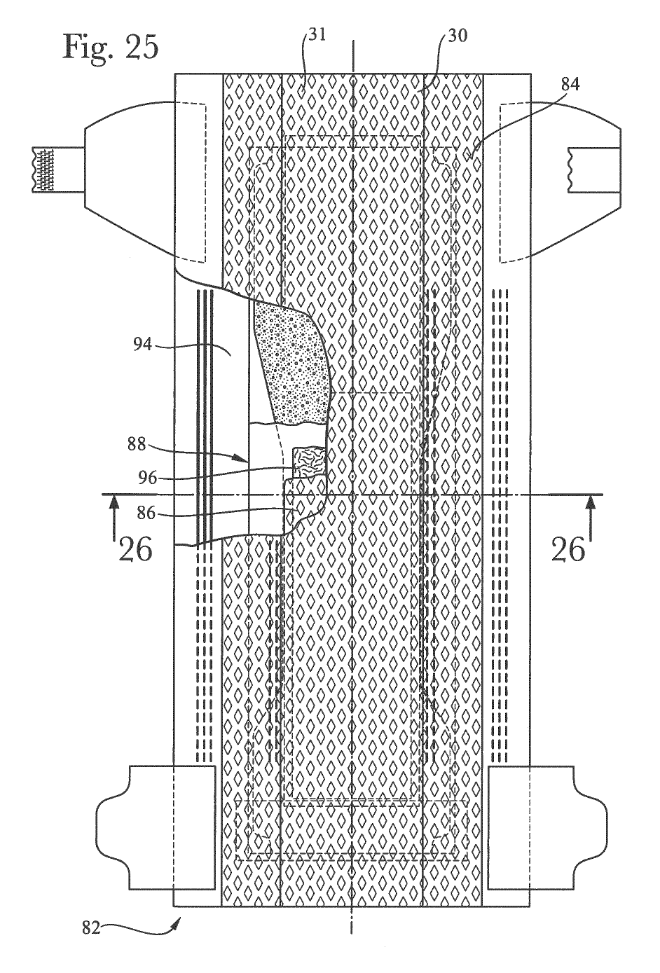

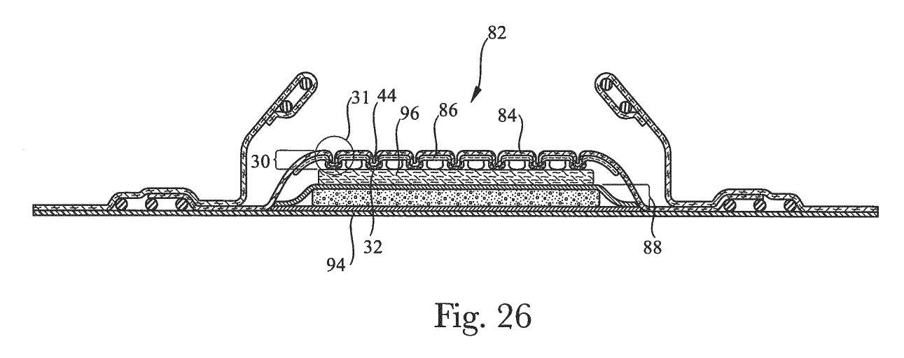

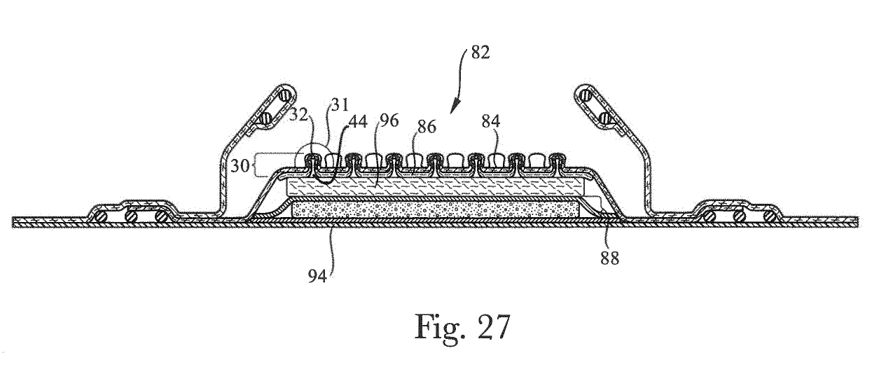

The nonwoven webs 30 described herein can comprise any suitable component or components of an absorbent article. For example, the nonwoven webs can comprise the topsheet of an absorbent article, or as shown in FIG. 25, if the nonwoven web 30 comprises more than one layer, the nonwoven web can comprise a combined topsheet 84 and acquisition layer 86 of an absorbent article, such as diaper 82. The diaper 82 shown in FIGS. 25-27 also comprises an absorbent core 88, a backsheet 94, and a distribution layer 96. The nonwoven materials of the present disclosure may also form an outer cover of an absorbent article, such as backsheet 94. The nonwoven webs 30 can be placed in an absorbent article with the deformations 31 in any suitable orientation. For example, the protrusions 32 can be oriented up or down. In other words, the protrusions 32 may be oriented toward the absorbent core 88 as shown in FIG. 26. Thus, for example, it may be desirable for the protrusions 32 to point inward toward the absorbent core 88 in a diaper (that is, away from the body-facing side and toward the garment-facing side), or other absorbent article. Alternatively, the protrusions 32 may be oriented so that they extend away from the absorbent core of the absorbent article as shown in FIG. 27. In still other embodiments, the nonwoven webs 30 can be made so that they have some protrusions 32 that are oriented upward, and some that are oriented downward. Without wishing to be bound to any particular theory, it is believed that such a structure may be useful in that the protrusions that are oriented upward can be more effective for cleaning the body from exudates, while the protrusions that are oriented downward can be more effective for absorption of exudates into the absorbent core. Therefore, without being bound to theory, a combination of these two protrusion orientations will offer advantage that the same product can fulfill the two functions.

A two or more layer nonwoven structure may provide fluid handling benefits. If the layers are integrated together, and the protrusions 32 are oriented toward the absorbent core, they may also provide a dryness benefit. It may be desirable, on the other hand, for the protrusions 32 to point outward, away from the absorbent core in a pad for a wet or dry mop to provide a cleaning benefit. In some embodiments, when the nonwoven web 30 is incorporated into an absorbent article, the underlying layers can be either substantially, or completely free, of tow fibers. Suitable underlying layers that are free of tow fibers may, for example, comprise a layer or patch of cross-linked cellulose fibers. In some cases, it may be desirable that the nonwoven material 30 is not entangled with (that is, is free from entanglement with) another web.

The layers of the nonwoven structure (e.g., a topsheet and/or acquisition layer) may be colored. Color may be imparted to the webs in any suitable manner including, but not limited to by color pigmentation. The term “color pigmentation” encompasses any pigments suitable for imparting a non-white color to a web. This term therefore does not include “white” pigments such as TiO₂ which are typically added to the layers of conventional absorbent articles to impart them with a white appearance. Pigments are usually dispersed in vehicles or substrates for application, as for instance in inks, paints, plastics or other polymeric materials. The pigments may for example be introduced in a polypropylene masterbatch. A masterbatch comprises a high concentration of pigment and/or additives which are dispersed in a carrier medium which can then be used to pigment or modify the virgin polymer material into a pigmented bicomponent nonwoven. An example of suitable colored masterbatch material that can be introduced is Pantone color 270 Sanylen violet PP 42000634 ex Clariant, which is a PP resin with a high concentration of violet pigment. Typically, the amount of pigments introduced by weight of the webs may be of from 0.3%-2.5%. Alternatively, color may be imparted to the webs by way of impregnation of a colorant into the substrate. Colorants such as dyes, pigments, or combinations may be impregnated in the formation of substrates such as polymers, resins, or nonwovens. For example, the colorant may be added to molten batch of polymer during fiber or filament formation.

Precursor Materials.

The nonwoven materials of the present disclosure can be made of any suitable nonwoven materials (“precursor materials”). The nonwoven webs can be made from a single layer, or multiple layers (e.g., two or more layers). If multiple layers are used, they can be comprised of the same type of nonwoven material, or different types of nonwoven materials. In some cases, the precursor materials may be free of any film layers.

The fibers of the nonwoven precursor material(s) can be made of any suitable materials including, but not limited to natural materials, synthetic materials, and combinations thereof. Suitable natural materials include, but are not limited to cellulose, cotton linters, bagasse, wool fibers, silk fibers, etc. Cellulose fibers can be provided in any suitable form, including but not limited to individual fibers, fluff pulp, drylap, liner board, etc. Suitable synthetic materials include, but are not limited to nylon, rayon and polymeric materials. Suitable polymeric materials include, but are not limited to: polyethylene (PE), polyester, polyethylene terephthalate (PET), polypropylene (PP), and co-polyester. In some embodiments, however, the nonwoven precursor materials can be either substantially, or completely free, of one or more of these materials. For example, in some embodiments, the precursor materials may be substantially free of cellulose, and/or exclude paper materials. In some embodiments, one or more precursor materials can comprise up to 100% thermoplastic fibers. The fibers in some cases may, therefore, be substantially non-absorbent. In some embodiments, the nonwoven precursor materials can be either substantially, or completely free, of tow fibers.

The precursor nonwoven materials can comprise any suitable types of fibers. Suitable types of fibers include, but are not limited to: monocomponent, bicomponent, and/or biconstituent, non-round (e.g., shaped fibers (including but not limited to fibers having a trilobal cross-section) and capillary channel fibers). The fibers can be of any suitable size. The fibers may, for example, have major cross-sectional dimensions (e.g., diameter for round fibers) ranging from 0.1-500 microns. Fiber size can also be expressed in denier, which is a unit of weight per length of fiber. The constituent fibers may, for example, range from about 0.1 denier to about 100 denier. The constituent fibers of the nonwoven precursor web(s) may also be a mixture of different fiber types, differing in such features as chemistry (e.g., PE and PP), components (mono- and bi-), shape (i.e. capillary channel and round) and the like.

The nonwoven precursor webs can be formed from many processes, such as, for example, air laying processes, wetlaid processes, meltblowing processes, spunbonding processes, and carding processes. The fibers in the webs can then be bonded via spunlacing processes, hydroentangling, calendar bonding, through-air bonding and resin bonding. Some of such individual nonwoven webs may have bond sites 46 where the fibers are bonded together.

In the case of spunbond webs, the web may have a thermal point bond 46 pattern that is not highly visible to the naked eye. For example, dense thermal point bond patterns are equally and uniformly spaced are typically not highly visible. After the material is processed through the mating male and female rolls, the thermal point bond pattern is still not highly visible. Alternatively, the web may have a thermal point bond pattern that is highly visible to the naked eye. For example, thermal point bonds that are arranged into a macro-pattern, such as a diamond pattern, are more visible to the naked eye. After the material is processed through the mating male and female rolls, the thermal point bond pattern is still highly visible and can provide a secondary visible texture element to the material.

The basis weight of nonwoven materials is usually expressed in grams per square meter (gsm). The basis weight of a single layer nonwoven material can range from about 8 gsm to about 100 gsm, depending on the ultimate use of the material 30. For example, the topsheet of a topsheet/acquisition layer laminate or composite may have a basis weight from about 8 to about 40 gsm, or from about 8 to about 30 gsm, or from about 8 to about 20 gsm. The acquisition layer may have a basis weight from about 10 to about 120 gsm, or from about 10 to about 100 gsm, or from about 10 to about 80 gsm. The basis weight of a multi-layer material is the combined basis weight of the constituent layers and any other added components. The basis weight of multi-layer materials of interest herein can range from about 20 gsm to about 150 gsm, depending on the ultimate use of the material 30. The nonwoven precursor webs may have a density that is between about 0.01 and about 0.4 g/cm³ measured at 0.3 psi (2 kPa).

The precursor nonwoven webs may have certain desired characteristics. The precursor nonwoven web(s) each have a first surface, a second surface, and a thickness. The first and second surfaces of the precursor nonwoven web(s) may be generally planar. It is typically desirable for the precursor nonwoven web materials to have extensibility to enable the fibers to stretch and/or rearrange into the form of the protrusions. If the nonwoven webs are comprised of two or more layers, it may be desirable for all of the layers to be as extensible as possible. Extensibility is desirable in order to maintain at least some non-broken fibers in the sidewalls around the perimeter of the protrusions. It may be desirable for individual precursor webs, or at least one of the nonwovens within a multi-layer structure, to be capable of undergoing an apparent elongation (strain at the breaking force, where the breaking force is equal to the peak force) of greater than or equal to about one of the following amounts: 100% (that is double its unstretched length), 110%, 120%, or 130% up to about 200%. It is also desirable for the precursor nonwoven webs to be capable of undergoing plastic deformation to ensure that the structure of the deformations is “set” in place so that the nonwoven web will not tend to recover or return to its prior configuration.

Materials that are not extensible enough (e.g., inextensible PP) may form broken fibers around much of the perimeter of the deformation, and create more of a “hanging chad” 90 (i.e., the cap 52 of the protrusions 32 may be at least partially broken from and separated from the rest of the protrusion (as shown in FIG. 20). The area on the sides of the protrusion where the fibers are broken is designated with reference number 92. Materials such as that shown in FIG. 20 will not be suitable for a single layer structure, and, if used, will typically be part of a composite multi-layer structure in which another layer has protrusions 32 as described herein.

When the fibers of a nonwoven web are not very extensible, it may be desirable for the nonwoven to be underbonded as opposed to optimally bonded. A thermally bonded nonwoven web's tensile properties can be modified by changing the bonding temperature. A web can be optimally or ideally bonded, underbonded, or overbonded. Optimally or ideally bonded webs are characterized by the highest breaking force and apparent elongation with a rapid decay in strength after reaching the breaking force. Under strain, bond sites fail and a small amount of fibers pull out of the bond site. Thus, in an optimally bonded nonwoven, the fibers 38 will stretch and break around the bond sites 46 when the nonwoven web is strained beyond a certain point. Often there is a small reduction in fiber diameter in the area surrounding the thermal point bond sites 46. Underbonded webs have a lower breaking force and apparent elongation when compared to optimally bonded webs, with a slow decay in strength after reaching the breaking force. Under strain, some fibers will pull out from the thermal point bond sites 46. Thus, in an underbonded nonwoven, at least some of the fibers 38 can be separated easily from the bond sites 46 to allow the fibers 38 to pull out of the bond sites and rearrange when the material is strained. Overbonded webs also have a lowered breaking force and elongation when compared to optimally bonded webs, with a rapid decay in strength after reaching the breaking force. The bond sites look like films and result in complete bond site failure under strain.

When the nonwoven web comprises two or more layers, the different layers can have the same properties, or any suitable differences in properties relative to each other. In one embodiment, the nonwoven web 30 can comprise a two layer structure that is used in an absorbent article. For convenience, the precursor webs and the material into which they are formed will generally be referred to herein by the same reference numbers. However, in some cases, for additional clarity the precursor web may be designated as 30′. As described above, one of the layers, a second layer 30B, can serve as the topsheet of the absorbent article, and the first layer 30A can be an underlying layer (or sub-layer) and serve as an acquisition layer. The acquisition layer 30A receives liquids that pass through the topsheet and distributes them to underlying absorbent layers. In such a case, the topsheet 30B may be less hydrophilic than sub-layer(s) 30A, which may lead to better dewatering of the topsheet. In other embodiments, the topsheet can be more hydrophilic than the sub-layer(s). In some cases, the pore size of the acquisition layer may be reduced, for example via using fibers with smaller denier or via increasing the density of the acquisition layer material, to better dewater the pores of the topsheet.

The second nonwoven layer 30B that may serve as the topsheet can have any suitable properties. Properties of interest for the second nonwoven layer, when it serves as a topsheet, in addition to sufficient extensibility and plastic deformation may include uniformity and opacity. As used herein, “uniformity” refers to the macroscopic variability in basis weight of a nonwoven web. As used, herein, “opacity” of nonwoven webs is a measure of the impenetrability of visual light, and is used as visual determination of the relative fiber density on a macroscopic scale. As used herein, “opacity” of the different regions of a single nonwoven deformation is determined by taking a photomicrograph at 20× magnification of the portion of the nonwoven containing the deformation against a black background. Darker areas indicate relatively lower opacity (as well as lower basis weight and lower density) than white areas.

Several examples of nonwoven materials suitable for use as the second nonwoven layer 30B include, but are not limited to: spunbonded nonwovens; carded nonwovens; and other nonwovens with high extensibility (apparent elongation in the ranges set forth above) and sufficient plastic deformation to ensure the structure is set and does not have significant recovery. One suitable nonwoven material as a topsheet for a topsheet/acquisition layer composite structure may be an extensible spunbonded nonwoven comprising polypropylene and polyethylene. The fibers can comprise a blend of polypropylene and polyethylene, or they can be bi-component fibers, such as a sheath-core fiber with polyethylene on the sheath and polypropylene in the core of the fiber. Another suitable material is a bi-component fiber spunbonded nonwoven comprising fibers with a polyethylene sheath and a polyethylene/polypropylene blend core.

The first nonwoven layer 30A that may, for example, serve as the acquisition layer can have any suitable properties. Properties of interest for the first nonwoven layer, in addition to sufficient extensibility and plastic deformation may include uniformity and opacity. If the first nonwoven layer 30A serves as an acquisition layer, its fluid handling properties must also be appropriate for this purpose. Such properties may include: permeability, porosity, capillary pressure, caliper, as well as mechanical properties such as sufficient resistance to compression and resiliency to maintain void volume. Suitable nonwoven materials for the first nonwoven layer when it serves as an acquisition layer include, but are not limited to: spunbonded nonwovens; through-air bonded (“TAB”) carded nonwoven materials; spunlace nonwovens; hydroentangled nonwovens; and, resin bonded carded nonwoven materials. Of course, the composite structure may be inverted and incorporated into an article in which the first layer 30A serves as the topsheet and the second layer 30B serves as an acquisition layer. In such cases, the properties and exemplary methods of the first and second layers described herein may be interchanged.

The layers of a two or more layered nonwoven web structure can be combined together in any suitable manner. In some cases, the layers can be unbonded to each other and held together autogenously (that is, by virtue of the formation of deformations therein). For example, both precursor webs 30A and 30B contribute fibers to deformations in a “nested” relationship that joins the two precursor webs together, forming a multi-layer web without the use or need for adhesives or thermal bonding between the layers. In other embodiments, the layers can be joined together by other mechanisms. If desired an adhesive between the layers, ultrasonic bonding, chemical bonding, resin or powder bonding, thermal bonding, or bonding at discrete sites using a combination of heat and pressure can be selectively utilized to bond certain regions or all of the precursor webs. In addition, the multiple layers may be bonded during processing, for example, by carding one layer of nonwoven onto a spunbond nonwoven and thermal point bonding the combined layers. In some cases, certain types of bonding between layers may be excluded. For example, the layers of the present structure may be non-hydroentangled together.

If adhesives are used, they can be applied in any suitable manner or pattern including, but not limited to: slots, spirals, spray, and curtain coating. Adhesives can be applied in any suitable amount or basis weight including, but not limited to between about 0.5 and about 30 gsm, alternatively between about 2 and about 5 gsm. Examples of adhesives could include hot melt adhesives, such as polyolefins and styrene block copolymers.

A certain level of adhesive may reduce the level of fuzz on the surface of the nonwoven material even though there may be a high percentage of broken fibers as a result of the deformation process. Glued dual-layer laminates produced as described herein are evaluated for fuzz. The method utilizes a Martindale Abrasion Tester, based upon ASTM D4966-98. After abrading the samples, they are graded on a scale of 1-10 based on the degree of fiber pilling (1=no fiber pills; 10=large quantity and size of fiber pills). The protrusions are oriented away from the abrader so the land area in between the depressions is the primary surface abraded. Even though the samples may have a significant amount of fiber breakage (greater than 25%, sometimes greater than 50%) in the side walls of the protrusions/depressions, the fuzz value may be low (around 2) for several different material combinations, as long as the layers do not delaminate during abrasion. Delamination is best prevented by glue basis weight, for example a glue basis weight greater than 3 gsm, and glue coverage.

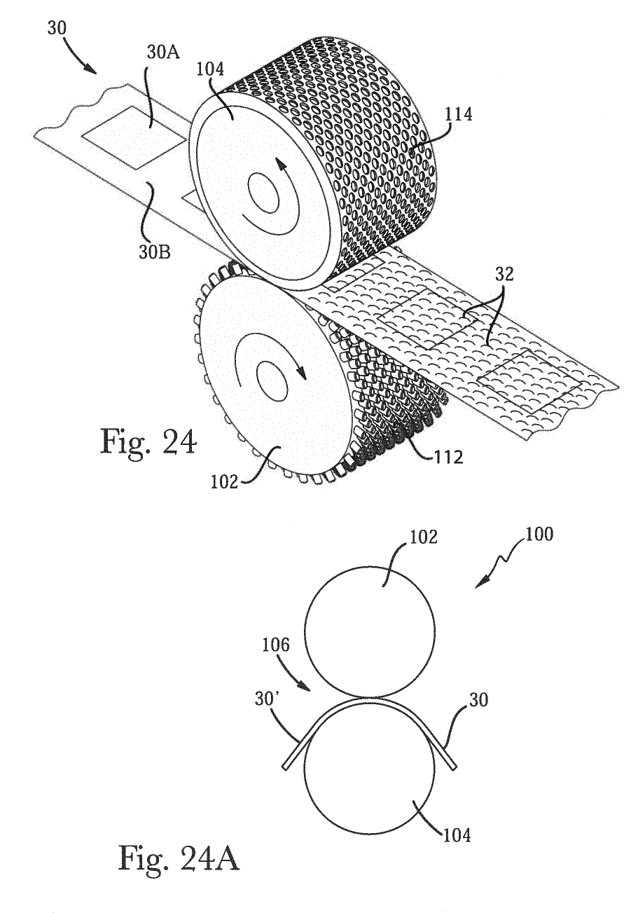

When the precursor nonwoven web comprises two or more layers, it may be desirable for at least one of the layers to be continuous, such as in the form of a web that is unwound from a roll. In some embodiments, each of the layers can be continuous. In alternative embodiments, such as shown in FIG. 24, one or more of the layers can be continuous, and one or more of the layers can have a discrete length. The layers may also have different widths. For example, in making a combined topsheet and acquisition layer for an absorbent article, the nonwoven layer that will serve as the topsheet may be a continuous web, and the nonwoven layer that will serve as the acquisition layer may be fed into the manufacturing line in the form of discrete length (for example, rectangular, or other shaped) pieces that are placed on top of the continuous web. Such an acquisition layer may, for example, have a lesser width than the topsheet layer. The layers may be combined together as described above.

### III. Methods of Making the Nonwoven Materials

The nonwoven materials are made by a method comprising the steps of: a) providing at least one precursor nonwoven web; b) providing an apparatus comprising a pair of forming members comprising a first forming member (a “male” forming member) and a second forming member (a “female” forming member); and c) placing the precursor nonwoven web(s) between the forming members and mechanically deforming the precursor nonwoven web(s) with the forming members. The forming members have a machine direction (MD) orientation and a cross-machine direction (CD) orientation.

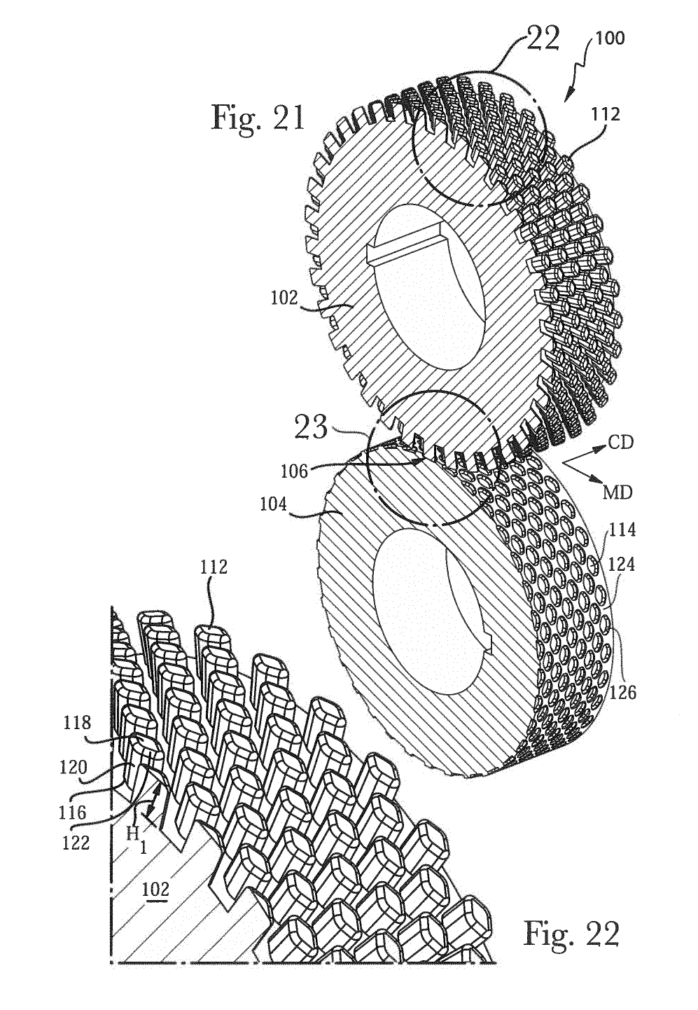

The first and second forming members can be plates, rolls, belts, or any other suitable types of forming members. In some embodiments, it may be desirable to modify the apparatus for incrementally stretching a web described in U.S. Pat. No. 8,021,591, Curro, et al. entitled “Method and Apparatus for Incrementally Stretching a Web” by providing the activation members described therein with the forming elements of the type described herein. In the embodiment of the apparatus 100 shown in FIG. 21, the first and second forming members 102 and 104 are in the form of non-deformable, meshing, counter-rotating rolls that form a nip 106 therebetween. The precursor web(s) is/are fed into the nip 106 between the rolls 102 and 104. Although the space between the rolls 102 and 104 is described herein as a nip, as discussed in greater detail below, in some cases, it may be desirable to avoid compressing the precursor web(s) to the extent possible.

First Forming Member.

The first forming member (such as “male roll”) 102 has a surface comprising a plurality of first forming elements which comprise discrete, spaced apart male forming elements 112. The male forming elements are spaced apart in the machine direction and in the cross-machine direction. The term “discrete” does not include continuous or non-discrete forming elements such as the ridges and grooves on corrugated rolls (or “ring rolls”) which have ridges that may be spaced apart in one, but not both, of the machine direction and in the cross-machine direction.

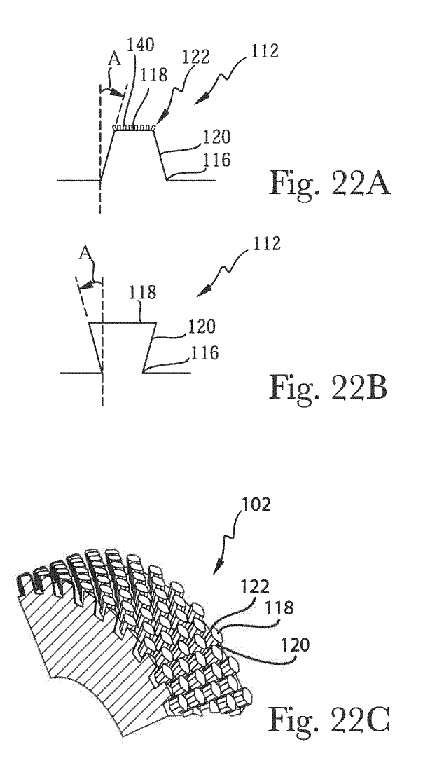

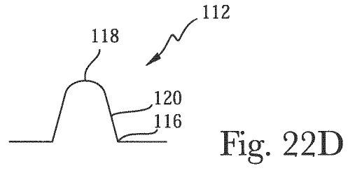

As shown in FIG. 22, the male forming elements 112 have a base 116 that is joined to (in this case is integral with) the first forming member 102, a top 118 that is spaced away from the base, and side walls (or “sides”) 120 that extend between the base 116 and the top 118 of the male forming elements. The male elements 112 may also have a transition portion or region 122 between the top 118 and the side walls 120. The male elements 112 also have a plan view periphery, and a height H₁ (the latter being measured from the base 116 to the top 118). The discrete elements on the male roll may have a top 118 with a relatively large surface area (e.g., from about 1 mm to about 10 mm in width, and from about 1 mm to about 20 mm in length) for creating a wide deformation. The male elements 112 may, thus, have a plan view aspect ratio (ratio of length to width) that ranges from about 1:1 to about 10:1. For the purpose of determining the aspect ratio, the larger dimension of the male elements 112 will be consider the length, and the dimension perpendicular thereto will be considered to be the width of the male element. The male elements 112 may have any suitable configuration.

The base 116 and the top 118 of the male elements 112 may have any suitable plan view configuration, including but not limited to: a rounded diamond configuration as shown in FIGS. 21 and 22, an American football-like shape, triangle, circle, clover, a heart-shape, teardrop, oval, or an elliptical shape. The configuration of the base 116 and the configuration of the top 118 of the male elements 112 may be in any of the following relationships to each other: the same, similar, or different. The top 118 of the male elements 112 can be flat, rounded, or any configuration therebetween.

The side walls 120 of the male elements 112 may have any suitable configuration. The male elements 112 may have vertical side walls 120, or tapered side walls 120. By vertical side walls, it is meant that the side walls 120 have zero degree side wall angles relative to the perpendicular from the base 116 of the side wall. In other embodiments, as shown in FIG. 22A, the side walls 120 can be tapered inwardly toward the center of the male forming elements 112 from the base 116 to the top 118 so that the side walls 120 form an angle, A, greater than zero. In still other embodiments, as shown in FIG. 22B, the male forming elements 112 may have a wider top surface than base so that the side walls 120 are angled outwardly away from the center of the male forming elements 112 from the base 116 to the top 118 of the male elements 112 (that is, the side walls may be undercut). The side wall angle can be the same on all sides of the male elements 112. Alternatively, the male elements 112 may have a different side wall angle on one or more of their sides. For example, the leading edge (or “LE”) and trailing edge (or “TE”) of the male elements (with respect to the machine direction) may have equal side wall angles, and the sides of the male elements may have equal side wall angles, but the side wall angles of the LE and TE may be different from the side wall angle of the sides. In certain embodiments, for example, the side wall angle of the sides of the male elements 112 may be vertical, and the side walls of the LE and TE may be slightly undercut.

The transition region or “transition” 122 between the top 118 and the side walls 120 of the male elements 112 may also be of any suitable configuration. The transition 122 can be in the form of a sharp edge (as shown in FIG. 22C) in which case there is zero, or a minimal radius where the side walls 120 and the top 118 of the male elements meet. That is, the transition 122 may be substantially angular, sharp, non-radiused, or non-rounded. In other embodiments, such as shown in FIG. 22, the transition 122 between the top 118 and the side walls 120 of the male elements 112 can be radiused, or alternatively beveled. Suitable radiuses include, but are not limited to: zero (that is, the transition forms a sharp edge), 0.01 inch (about 0.25 mm), 0.02 inch (about 0.5 mm), 0.03 inch (about 0.76 mm), 0.04 inch (about 1 mm) (or any 0.01 inch increment above 0.01 inch), up to a fully rounded male element as shown in FIG. 22D.

Numerous other embodiments of the male forming elements 112 are possible. In other embodiments, the top 118 of the male elements 112 can be of different shapes from those shown in the drawings. In other embodiments, the male forming elements 112 can be disposed in other orientations on the first forming member 102 rather than having their length oriented in the machine direction (including CD-orientations, and orientations between the MD and CD). The male forming elements 112 on the first forming member 102 may, but need not, all have the same configuration or properties. In certain embodiments, the first forming member 102 can comprise some male forming elements 112 having one configuration and/or properties, and other male forming elements 112 having one or more different configurations and/or properties.

The method of making the nonwoven materials may be run with the first forming member 102 and male elements 112 under any of the following conditions: at room temperature; with a chilled first forming member 102 and/or male elements 112; or with heated first forming member and/or male elements. In some cases, it may be desired to avoid heating the first forming member 102 and/or male elements 112. It may be desirable to avoid heating the first forming member and/or the male elements altogether. Alternatively, it may be desirable to avoid heating the first forming member and/or the male elements to a temperature at or above that which would cause the fibers of the nonwoven to fuse together. In some cases, it may be desirable to avoid heating the first forming member and/or the male elements to a temperature that is greater than or equal to any of the following temperatures: 130° C., 110° C., 60° C., or greater than 25° C.

Second Forming Member.

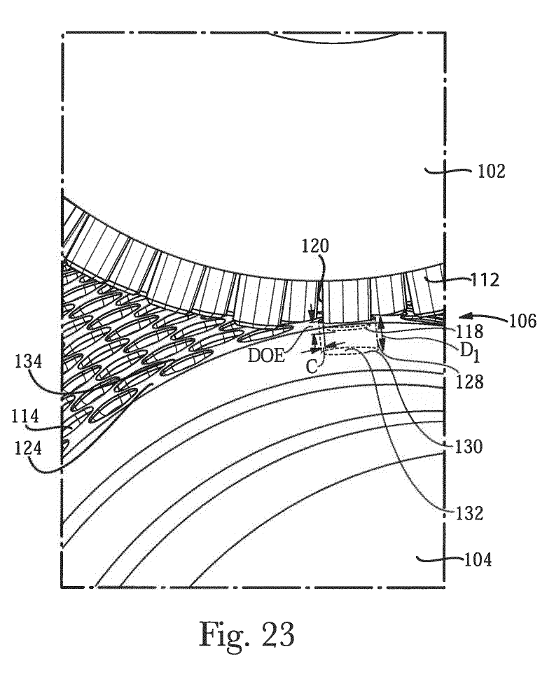

As shown in FIG. 21, the second forming member (such as “female roll”) 104 has a surface 124 having a plurality of cavities or recesses 114 therein. The recesses 114 are aligned and configured to receive the male forming elements 112 therein. Thus, the male forming elements 112 mate with the recesses 114 so that a single male forming element 112 fits within the periphery of a single recess 114, and at least partially within the recess 114 in the z-direction. The recesses 114 have a plan view periphery 126 that is larger than the plan view periphery of the male elements 112. As a result, the recess 114 on the female roll may completely encompass the discrete male element 112 when the rolls 102 and 104 are intermeshed. The recesses 114 have a depth D₁ shown in FIG. 23. In some cases, the depth D₁ of the recesses may be greater than the height H₁ of the male forming elements 112.

The recesses 114 have a plan view configuration, side walls 128, a top edge or rim 134 around the upper portion of the recess where the side walls 128 meet the surface 124 of the second forming member 104, and a bottom edge 130 around the bottom 132 of the recesses where the side walls 128 meet the bottom 132 of the recesses.

The recesses 114 may have any suitable plan view configuration provided that the recesses can receive the male elements 112 therein. The recesses 114 may have a similar plan view configuration as the male elements 112. In other cases, some or all of the recesses 114 may have a different plan view configuration from the male elements 112.

The side walls 128 of the recesses 114 may be oriented at any suitable angle. In some cases, the side walls 128 of the recesses may be vertical. In other cases, the side walls 128 of the recesses may be oriented at an angle. Typically, this will be an angle that is tapered inwardly from the top 134 of the recess 114 to the bottom 132 of the recess. The angle of the side walls 128 of the recesses can, in some cases, be the same as the angle of the side walls 120 of the male elements 112. In other cases, the angle of the side walls 128 of the recesses can differ from the angle of the side walls 120 of the male elements 112.

The top edge or rim 134 around the upper portion of the recess where the side walls 128 meet the surface 124 of the second forming member 104 may have any suitable configuration. The rim 134 can be in the form of a sharp edge (as shown in FIG. 23) in which case there is zero, or a minimal radius where the side walls 128 of the recesses meet the surface of the second forming member 104. That is, the rim 134 may be substantially angular, sharp, non-radiused, or non-rounded. In other embodiments, the rim 134 can be radiused, or alternatively beveled. Suitable radiuses include, but are not limited to: zero (that is, form a sharp edge), 0.01 inch (about 0.25 mm), 0.02 inch (about 0.5 mm), 0.03 inch (about 0.76 mm), 0.04 inch (about 1 mm) (or any 0.01 inch increment above 0.01 inch) up to a fully rounded land area between some or all of the side walls 128 around each recess 114. The bottom edge 130 of the recesses 114 may be sharp or rounded.

As discussed above, the recesses 114 may be deeper than the height H₁ of the male elements 112 so the nonwoven material is not nipped (or squeezed) between the male and female rolls 102 and 104 to the extent possible. However, it is understood that passing the precursor web(s) between two rolls with a relatively small space therebetween will likely apply some shear and compressive forces to the web(s). The present method, however, differs from some embossing processes in which the top of the male elements compress the material to be embossed against the bottom of the female elements, thereby increasing the density of the region in which the material is compressed.

The depth of engagement (DOE) is a measure of the level of intermeshing of the forming members. As shown in FIG. 23, the DOE is measured from the top 118 of the male elements 112 to the (outermost) surface 124 of the female forming member 114 (e.g., the roll with recesses). The DOE should be sufficiently high, when combined with extensible nonwoven materials, to create protrusions 32 having a distal portion or cap 52 with a maximum width that is greater than the width of the base opening 44. The DOE may, for example, range from at least about 1.5 mm, or less, to about 5 mm, or more. In certain embodiments, the DOE may be between about 2.5 mm to about 5 mm, alternatively between about 3 mm and about 4 mm. The formation of protrusions 32 having a distal portion with a maximum width that is greater than the width of the base opening 44 is believed to differ from most embossing processes in which the embossments typically take the configuration of the embossing elements, which have a base opening that is wider than the remainder of the embossments.

As shown in FIG. 23, there is a clearance, C, between the sides 120 of the male elements 112 and the sides (or side walls) 128 of the recesses 114. The clearances and the DOE's are related such that larger clearances can permit higher DOE's to be used. The clearance, C, between the male and female roll may be the same, or it may vary around the perimeter of the male element 112. For example, the forming members can be designed so that there is less clearance between the sides of the male elements 112 and the adjacent side walls 128 of the recesses 114 than there is between the side walls at the end of the male elements 112 and the adjacent side walls of the recesses 114. In other cases, the forming members can be designed so that there is more clearance between the sides 120 of the male elements 112 and the adjacent side walls 128 of the recesses 114 than there is between the side walls at the end of the male elements 112 and the adjacent side walls of the recesses. In still other cases, there could be more clearance between the side wall on one side of a male element 112 and the adjacent side wall of the recess 114 than there is between the side wall on the opposing side of the same male element 112 and the adjacent side wall of the recess. For example, there can be a different clearance at each end of a male element 112; and/or a different clearance on each side of a male element 112. Clearances can range from about 0.005 inches (about 0.1 mm) to about 0.1 inches (about 2.5 mm).

Some of the aforementioned male element 112 configurations alone, or in conjunction with the second forming member 104 and/or recess 114 configurations may provide additional advantages. This may be due to by greater lock of the nonwoven material on the male elements 112, which may result in more uniform and controlled strain on the nonwoven precursor material. This may produce more well-defined protrusions 32 and a stronger visual signal for consumers, giving the appearance of softness, absorbency, and/or dryness.

The precursor nonwoven web 30 is placed between the forming members 102 and 104. The precursor nonwoven web can be placed between the forming members with either side of the precursor web (first surface 34 or second surface 36) facing the first forming member, male forming member 102. For convenience of description, the second surface 36 of the precursor nonwoven web will be described herein as being placed in contact with the first forming member 102. (Of course, in other embodiments, the second surface 36 of the precursor nonwoven web can be placed in contact with the second forming member 104.)

The precursor material is mechanically deformed with the forming members 102 and 104 when a force is applied on the nonwoven web with the forming members 102 and 104. The force can be applied in any suitable manner. If the forming members 102 and 104 are in the form of plates, the force will be applied when the plates are brought together. If the forming members 102 and 104 are in the form of counter-rotating rolls (or belts, or any combination of rolls and belts), the force will be applied when the precursor nonwoven web passes through the nip between the counter-rotating elements. The force applied by the forming members impacts the precursor web and mechanically deforms the precursor nonwoven web.

Numerous additional processing parameters are possible. If desired, the precursor nonwoven web may be heated before it is placed between the forming members 102 and 104. If the precursor nonwoven web is a multi-layer structure, any layer or layers of the same can be heated before the layers are combined. Alternatively, the entire multi-layer nonwoven web can be heated before it is placed between the forming members 102 and 104. The precursor nonwoven web, or layer(s) of the same, can be heated in any suitable manner including, but not limited to using conductive heating (such as by bringing the web(s) in contact with heated rolls), or by convective heating (i.e., by passing the same under a hot air knife or through an oven). The heating should be non-targeted, and without the help of any agent. The first forming member 102 and/or second forming member 104 (or any suitable portion thereof) can also be heated. If desired, the web could be additionally, or alternatively, heated after it is mechanically deformed.

If the precursor material is fed between forming members comprising counter-rotating rolls, several processing parameters may be desirable. With regard to the speed at which the precursor web is fed between the counter-rotating rolls, it may be desirable to overfeed the web (create a negative draw) going into the nip 106 between the rolls. The surface speed of the metering roll immediately upstream of the forming members 102 and 104 may be between about 1 and 1.2 times the surface speed of the forming members 102 and 104. It may be desirable for the tension on the precursor web immediately before forming members 102 and 104 to be less than about 5 lbs. force (about 22 N), alternatively less than about 2 lbs. force (about 9 N) for a web width of 0.17 m. With regard to the speed at which the deformed web 30 is removed from between the counter-rotating rolls, it may be desirable to create a positive draw coming out of the nip between the rolls. The surface speed of the metering roll immediately downstream of the forming members 102 and 104 may be between about 1 and 1.2 times the surface speed of the forming members 102 and 104. It may be desirable for the tension on the web immediately after the forming members 102 and 104 to be less than about 5 lbs. force (about 22 N), alternatively less than about 2 lbs. force (about 9 N).

As shown in FIG. 24A, rather than feeding the precursor web 30′ into the nip 106 between the forming members 102 and 104 without the precursor web 30′ contacting any portion of the forming members prior to or after the nip, it may be desirable for the web to pre-wrap the second forming member 104 prior to entering the nip 106, and for the web 30 to post wrap second forming member 104 after passing through the nip.

The apparatus 100 for deforming the web can comprise multiple nips for deforming portions of the web in the same location such as described in U.S. Patent Publication No. US 2012/0064298 A1, Orr, et al. For example, the apparatus may comprise a central roll and satellite rolls with equal DOE or progressively greater DOE with each successive roll. This can provide benefits such as reducing damage to the web and/or helping to further ensure that the deformations are permanently set in the web thereby preventing the web from recovering toward its undeformed condition.

The apparatus for deforming the web can also comprise belts, or other mechanisms, for holding down the longitudinal edges of the web to prevent the web from being drawn inward in the cross-machine direction.

When deforming multiple webs that are laminated together with an adhesive, it may be desirable to chill the forming members in order to avoid glue sticking to and fouling the forming members. The forming members can be chilled using processes know in the art. One such process could be an industrial chiller that utilizes a coolant, such as propylene glycol. In some cases, it may be desirable to operate the process in a humid environment such that a layer of condensate forms on the forming members.

The apparatus 100 for deforming the web can be at any suitable location in any suitable process. For example, the apparatus can be located in-line with a nonwoven web making process or a nonwoven laminate making process. Alternatively, the apparatus 100 can be located in-line in an absorbent article converting process (such as after the precursor web is unwound and before it is incorporated as part of the absorbent article).

The process forms a nonwoven web 30 comprising a generally planar first region 40 and a plurality of discrete integral second regions 42 that comprise deformations comprising protrusions 32 extending outward from the first surface 34 of the nonwoven web and openings in the second surface 36 of the nonwoven web. (Of course, if the second surface 36 of the precursor nonwoven web is placed in contact with the second forming member 104, the protrusions will extend outward from the second surface of the nonwoven web and the openings will be formed in the first surface of the nonwoven web.) Without wishing to be bound by any particular theory, it is believed that the extensibility of the precursor web (or at least one of the layers of the same) when pushed by the male forming elements 112 into the recesses 114 with depth of engagement DOE being less than the depth D₁ of the recesses, stretches a portion of the nonwoven web to form a deformation comprising a protrusion with the enlarged cap and wide base opening described above. (This can be analogized to sticking one's finger into an uninflated balloon to stretch and permanently deform the material of the balloon.)

In cases in which the precursor nonwoven material 30′ comprises more than one layer, and one of the layers is in the form of discrete pieces of nonwoven material, as shown in FIG. 24, it may be desirable for the deformations to be formed so that the base openings 44 are in the continuous layer (such as 30B) and the protrusions 32 extend toward the discrete layer (such as 30A). Of course, in other embodiments, the deformations in such a structure can be in the opposite orientation. The deformations can be distributed in any suitable manner over the surfaces of such continuous and discrete layers. For example, the deformations can: be distributed over the full length and/or width of the continuous layer; be distributed in an area narrower than the width of the continuous layer; or be limited to the area of the discrete layer.

In some instances, the ratio of the circumference of the protrusions (loop circumference length) to the length of the second surface opening 64 (see FIG. 11) is less than 4:1. To measure the loop circumference length, arrange the web comprising the protrusion so that the viewing direction is co-linear with the longitudinal axis (MD) of the protrusion. Adjust the magnification so that one protrusion is completely in view. If necessary, a cross-section of the protrusion may be obtained by cutting the protrusion perpendicular to the longitudinal axis using sharp scissors or a razor blade, taking care in preserving the overall geometry of the protrusion while cutting it. Referring to FIG. 12, measure and record the loop circumference length by starting the measurement at the first origination point A, proceeding along the median path of the loop fibers B, and terminating the measurement at the second origination point C. Measure and record the base length of the second surface opening 64, parallel to the plane of the web between the first origination point A and the second origination point C. The protrusion base length of the second surface opening 64 is measured parallel to the plane of the web and may be at the plane of the web or above the plane of the web. The protrusions are measured where the protrusions are not under any pressure or strain.

### IV. Apertures in the Nonwoven Material and Absorbent Articles Comprising the Nonwoven Material Having Apertures

A plurality of apertures may be formed in the nonwoven material. The nonwoven material may have one or more layers. In one example, the nonwoven material may be a topsheet and acquisition layer of an absorbent article, for example. Apertures may be formed through all of, or through one or more of these layers in the nonwoven material. The apertures may be coincident if formed through one or more of the layers or all of the layers. The apertures may be formed in portions of the generally planar first region and/or in at least some of, or all of, the discrete integral second regions in all of the layers of the nonwoven material or in some of the layers of the nonwoven material. The apertures in the nonwoven material may be formed in a predetermined and intentional pattern. Stated another way, the apertures are not merely unintentional variances in the nonwoven material or unintentional tears formed during manufacturing, such as the unintentional tears illustrated in FIGS. 15C-15F, for example. In a form, the apertures further may form a uniform, repeating pattern, wherein the distance between apertures (CD or MD) is the same or substantially the same. In other forms, the apertures may have a non-regular repeating pattern with interaperture distances (i.e., distances between the apertures) that may be variable within, for example, a certain absorbent article, if the nonwoven material is used as a topsheet and/or acquisition layer, or topsheet and acquisition layer laminate, in the absorbent article. A second absorbent article may have the same non-regular repeating pattern in the nonwoven material.

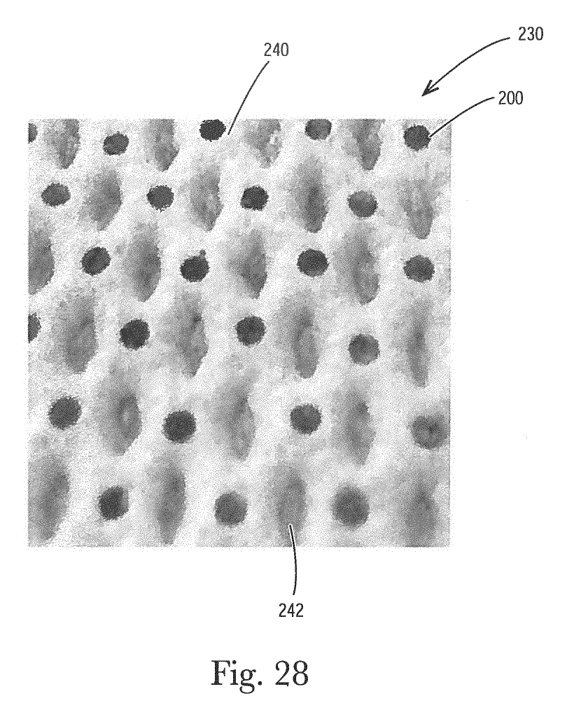

Referring to FIG. 28, an example nonwoven material 230 has apertures 200 defined in generally planar first regions 240. The discrete integral second regions 242 do not have apertures, but may have unintentional tears, like those illustrated in FIGS. 15C-15F. Although the discrete integral second regions 242 are illustrated as downwardly facing in FIG. 28, they may also be upwardly facing, as illustrated in FIG. 5 and as described herein. The nonwoven material 230 may comprise one or more layers. In the illustrated nonwoven material 230, a first layer may be a topsheet and a second layer may be an acquisition layer of an absorbent article, for example. The apertures 200 may be registered with the generally planar first regions 240, as will be discussed in further detail below. The apertures 200 may be formed in the generally planar first regions 240 in a predetermined, intentional pattern, such that the apertures 200 have a substantially uniform spacing (e.g., not a random pattern of apertures). The apertures 200 may have any suitable size, shape, and/or orientation. In some instances, the apertures 200 may be planned (i.e., intended manufacture) to have generally have the same size, shape, and/or orientation, although those of skill in the art will recognize variances in materials, apertures size, aperture shape, and/or aperture orientation. The apertures 200 may have similar, substantially similar, or the same aspect ratios. Having the apertures 200 in the planar first regions 240 allows for better BM, or other bodily fluid, acquisition over the three-dimensional wearer-facing surface (in an absorbent article context) and in voids created below the apertures 200. The voids may be formed between the generally planar first regions 240 and the next flat layer underneath the nonwoven material (e.g., a core). By having these apertures 200 and voids, BM, or other bodily fluids, are easily able to bypass some of the resistance to acquisition of the topsheet, thereby reducing BM, or other bodily fluid, spreading (i.e., run-off). The apertures 200 also allow the topsheet to acquire urine better while being less hydrophilic than typical topsheets, or hydrophobic thereby leading to better dryness, especially with relatively large aperture dimensions (e.g., greater than 0.75 mm in width and/or length, greater than 1.0 mm in width and/or length, greater than 1.5 mm in width and/or length, or greater than 2.0 mm in width and/or length, for example. This dryer wearer-facing surface may also lead to reduced skin marking or red marking.

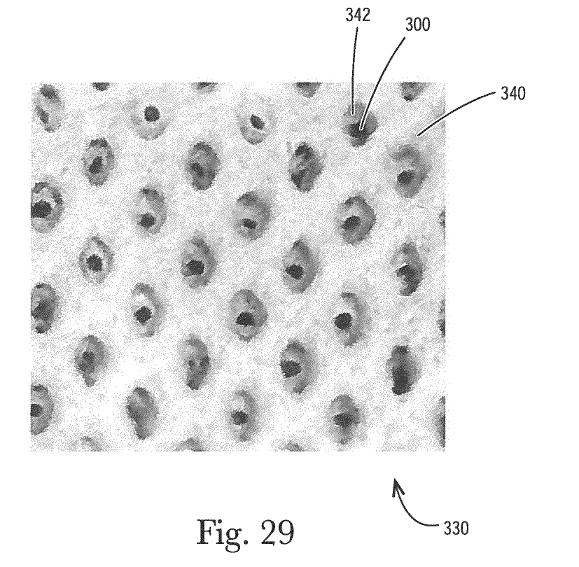

Referring to FIG. 29, an example nonwoven material 330 has apertures 300 defined in discrete integral second regions 342 while generally planar first regions 340 are free of apertures. The apertures 300 may be defined in the distal end, side walls, and/or cap of the discrete integral second regions 342. In addition to the apertures 300, at least some portions of the discrete integral second regions 342 may also have unintentional tears, like those illustrated in FIGS. 15C-15F. Although the discrete integral second regions 342 are illustrated as downwardly facing in FIG. 29, they may also be upwardly facing, as illustrated in FIG. 5 and as described herein. The nonwoven material 330 may comprise one or more layers. In the illustrated nonwoven material 330, a first layer may be a topsheet and a second layer may be an acquisition layer of an absorbent article, for example. The apertures 300 may be registered with the discrete integral second regions 342 as will be discussed in further detail below. The apertures 300 may be formed in the discrete integral second regions 342 in a predetermined, intentional pattern, such that the apertures 300 have a substantially uniform spacing therebetween (e.g., not a random pattern of apertures). The apertures 300 may have any suitable size, shape, and/or orientation. In some instances, the apertures 300 may be planned (i.e., intended manufacture) to have generally have the same size, shape, and/or orientation, although those of skill in the art will recognize variances in materials, apertures size, aperture shape, and/or aperture orientation. The apertures 300 may have similar, substantially similar, or the same aspect ratios. Having the apertures 300 in the discrete integral second regions 342 allows for better BM, or other bodily fluid, acquisition over the three-dimensional wearer-facing surface (in an absorbent article context). By having these apertures 300, BM, or other bodily fluids, are easily able to bypass some of the resistance to acquisition of the topsheet, thereby reducing BM, or other bodily fluid, spreading (i.e., run-off) (especially when the BM, or other bodily fluids, are within the discrete integral second regions 342). The apertures 300 also allow the topsheet to acquire urine better while being less hydrophilic than typical topsheets, or hydrophobic, thereby leading to better dryness, especially with relatively large aperture dimensions (e.g., greater than 0.75 mm in width and/or length, greater than 1.0 mm in width and/or length, greater than 1.5 mm in width and/or length, or greater than 2.0 mm in width and/or length, for example. This dryer wearer-facing surface may also lead to reduced skin marking or red marking. As the apertures 300 are located at distal ends of the discrete integral second regions 342 and not in contact with a wearer's skin, the apertures 300 may lead to softness improvements in the nonwoven material 330. If the discrete integral second regions 342 are downwardly facing (e.g., extending towards an absorbent core of an absorbent article), they may create large void volumes with an apertures at the distal end. These apertures 300 may act as a drain to the large void volumes to channel bodily exudates towards an absorbent core. Bodily exudates may be quickly absorbed through the apertures in view of the highly permeable layers below them (e.g., a distribution layer or acquisition layer).

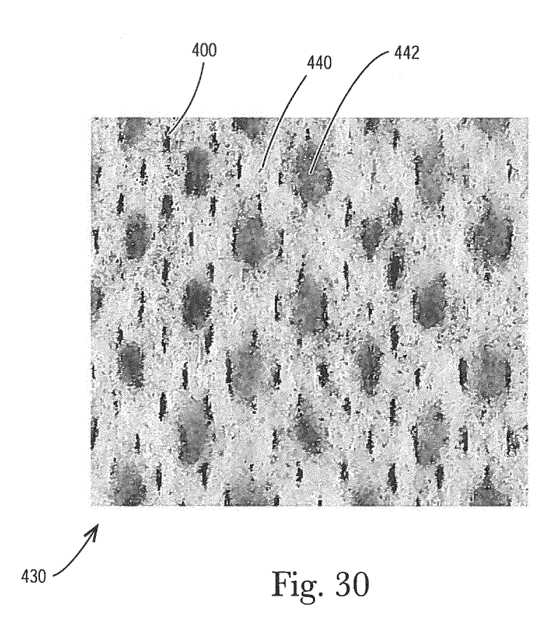

Referring to FIG. 30, an example nonwoven material 430 has unregistered apertures 400 defined in generally planar first regions 440 and in the discrete integral second regions 442. The discrete integral second regions 442 may also have unintentional tears, like those illustrated in FIGS. 15C-15F. Although the discrete integral second regions 442 are illustrated as downwardly facing in FIG. 30, they may also be upwardly facing, as illustrated in FIG. 5 and as described herein. The nonwoven material 430 may comprise one or more layers. In the illustrated nonwoven material 430, a first layer may be a topsheet and a second layer may be an acquisition layer of an absorbent article, for example. The apertures 400 may be formed in one or more layers in a predetermined, intentional pattern, such that the apertures 200 have a substantially uniform spacing (e.g., not a random pattern of apertures). The apertures 400 may have any suitable size, shape, and/or orientation. In some instances, the apertures 400 may be planned to have generally have the same size, shape, and/or orientation, although those of skill in the art will recognize variances in materials, apertures size, aperture shape, and/or aperture orientation. The apertures 400 may have similar, substantially similar, or the same aspect ratios. Having the apertures 400 being not registered with only the planar first regions 440 or only the discrete integral second regions 442 allows for better BM, or other bodily fluid, acquisition over the three-dimensional wearer-facing surface (in an absorbent article context) and in voids created below the apertures 400. The voids may be formed between the generally planar first regions 440 and the next flat layer underneath the nonwoven material (e.g., a core). By having these apertures 400 and voids, BM, or other bodily fluids, are easily able to bypass some of the resistance to acquisition of the topsheet, thereby reducing BM, or other bodily fluid, spreading (i.e., run-off). The apertures 400 also allow the topsheet to acquire urine better while being less hydrophilic than typical topsheets, or hydrophobic, thereby leading to better dryness, especially with relatively large aperture dimensions (e.g., greater than 0.75 mm in width and/or length, greater than 1.0 mm in width and/or length, greater than 1.5 mm in width and/or length, or greater than 2.0 mm in width and/or length, for example. This dryer wearer-facing surface may also lead to reduced skin marking or red marking.

In various forms, apertures in the generally planar first regions may be smaller than apertures in the discrete integral second regions. Smaller apertures in the generally planar first regions may be desired for purposes of reduced rewet and softness, since these apertures may be in contact with an absorbent article wearer. Larger apertures in the discrete integral second regions may be desired for fluid handling and do not have rewet and softness issues, since these larger apertures are not in contact with an absorbent article wearer.

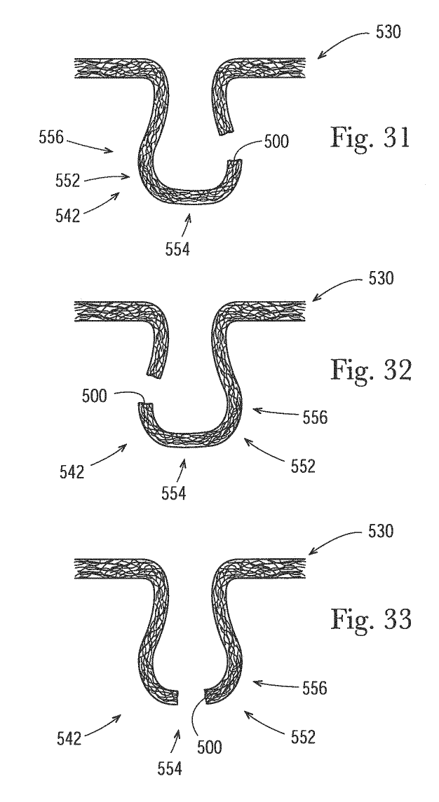

For FIGS. 31-44, the apertures may have any of the features described above with respect to FIGS. 28-30. Also for FIGS. 31-44, the discrete integral second regions are illustrated as oriented down (e.g., in an absorbent article context, extending toward the absorbent core), but they may also be oriented up, as described above with reference to FIG. 5.

Referring to FIGS. 31-33, schematic illustrations of a single layer nonwoven material 530 are illustrated. The single layer of nonwoven material 530 may have intentional apertures 500 formed in side walls 556 and/or caps 552 (FIGS. 31 and 32) of the discrete integral second regions 542 or in distal ends 554 (FIG. 33) of the discrete integral second regions 542. In some instances, the apertures 500 may be formed at least partially in the distal ends 554 and at least partially in the side walls 556 and/or the caps 552.

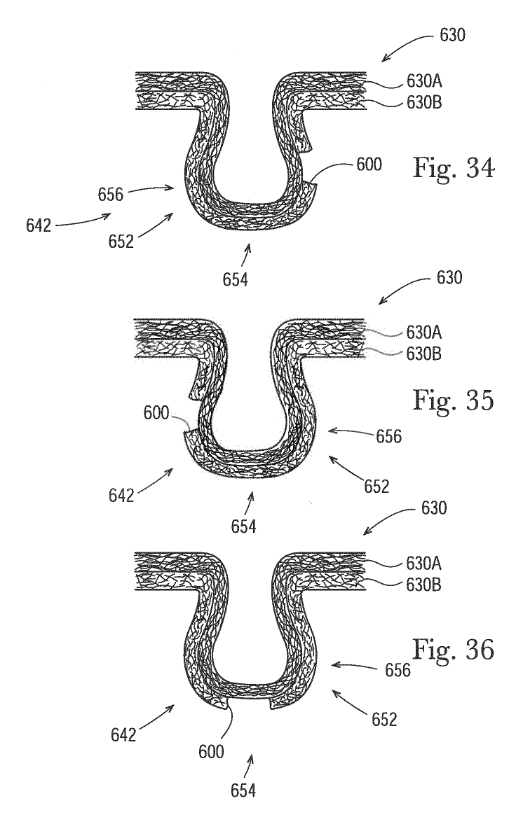

Referring to FIGS. 34-36, schematic illustrations of a dual layer nonwoven material 630 are illustrated. A top layer 630A of the nonwoven material 630 may not comprise apertures, while a bottom layer 630B may have intentional apertures 600 formed in side walls 656 and/or caps 652 (FIGS. 34 and 35) of discrete integral second regions 642 or in distal ends 654 (FIG. 36) of the discrete integral second regions 642. In some instances, the apertures 600 may be formed at least partially in the distal ends 654 and at least partially in the side walls 656 and/or the caps 652. The apertures 600 in the bottom layers 630B may allow for faster urine, or other bodily fluid, acquisition into layers beneath the second layer (e.g., a core of an absorbent article).

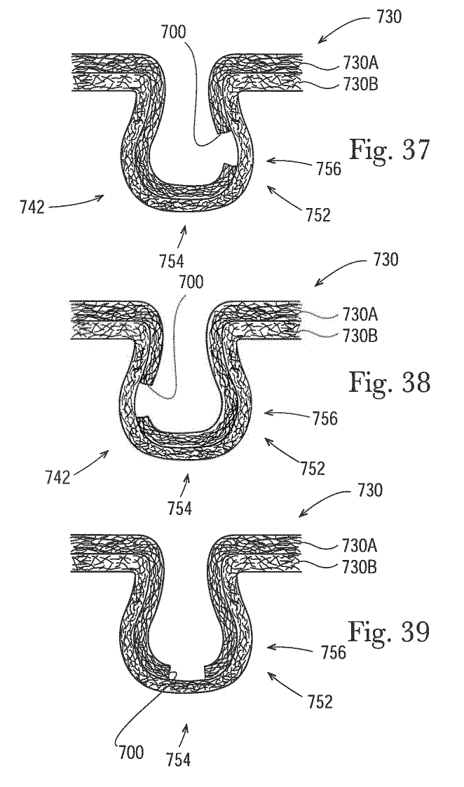

Referring to FIGS. 37-39, schematic illustrations of a dual layer nonwoven material 730 are illustrated. A bottom layer 730B of the nonwoven material 730 may not comprise apertures, while a top layer 730A may have intentional apertures 700 formed in side walls 756 and/or caps 752 (FIGS. 37 and 38) of the discrete integral second regions 742 or in distal ends 754 (FIG. 39) of the discrete integral second regions 742. In some instances, the apertures 700 may be formed at least partially in the distal ends 754 and at least partially in the side walls 756 and/or the caps 752. By having these apertures 700 in the top layer 730A, BM, or other bodily fluids, are easily able to bypass some of the resistance to acquisition of the topsheet (e.g., top layer), thereby reducing BM, or other bodily fluid, spreading (i.e., run-off) (especially when the BM, or other bodily fluids are within the discrete integral second regions 742). The apertures 700 also allow the topsheet to acquire urine better while being less hydrophilic than typical topsheets, or hydrophobic, thereby leading to better dryness, especially with relatively large aperture dimensions (e.g., greater than 0.75 mm in width and/or length, greater than 1.0 mm in width and/or length, greater than 1.5 mm in width and/or length, or greater than 2.0 mm in width and/or length, for example. This dryer wearer-facing surface may also lead to reduced skin marking or red marking. As the apertures 700 are located in the discrete integral second regions 742 and not in contact with a wearer's skin, the apertures 700 may lead to softness improvements in the nonwoven material 730. By having apertures 700 only in the first layer 730A, the apertures 700 are less prone to collapse/closure and the non-apertured second layer 730B may help inhibit rewet and mask bodily exudates beneath it (e.g., in an absorbent core)

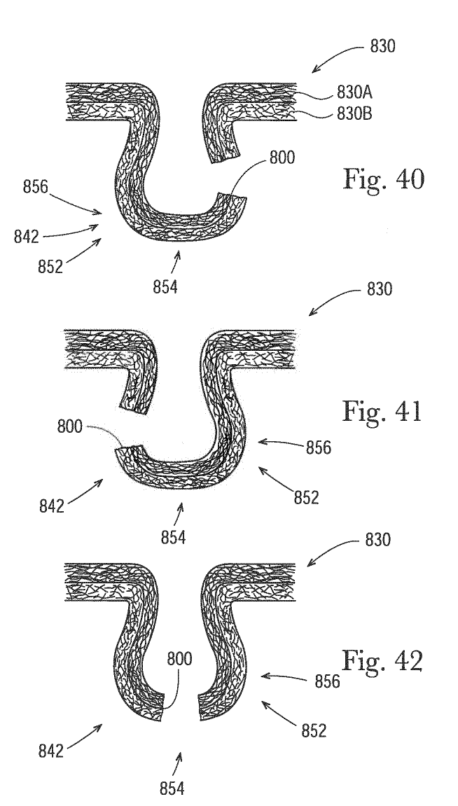

Referring to FIGS. 40-42, schematic illustrations of a dual layer nonwoven material 830 are illustrated. A top layer 830a of the nonwoven material 830 may comprise intentional apertures 800 and a bottom layer 830B may have intentional apertures 800. The apertures 800 may be coincident and may be formed in side walls 856 and/or caps 852 (FIGS. 40 and 41) of the discrete integral second regions 842 or in distal ends 854 (FIG. 42) of the discrete integral second regions 842. In some instances, the apertures 800 may be formed at least partially in the distal ends 854 and at least partially in the side walls 856 and/or the caps 852. By providing apertures 800 through both layers of the nonwoven material 830, BM and bodily fluids may be better absorbed and, in an absorbent article context, wicked toward an absorbent core. By having these apertures 800 in the top layer 830A and the bottom layer 830B, BM, or other bodily fluids, are easily able to bypass some of the resistance to acquisition of the topsheet (e.g., top layer) and the acquisition layer (e.g., bottom layer), thereby reducing BM, or other bodily fluid, spreading (i.e., run-off) (especially when the BM, or other bodily fluids are within the discrete integral second regions 842). The apertures 800 also allow the topsheet to acquire urine better while being less hydrophilic or hydrophobic than typical topsheets, thereby leading to better dryness, especially with relatively large aperture dimensions (e.g., greater than 0.75 mm in width and/or length, greater than 1.0 mm in width and/or length, greater than 1.5 mm in width and/or length, or greater than 2.0 mm in width and/or length, for example. This dryer wearer-facing surface may also lead to reduced skin marking or red marking. As the apertures 800 are located in the discrete integral second regions 742 and not in contact with a wearer's skin, the apertures 800 may lead to softness improvements in the nonwoven material 830.

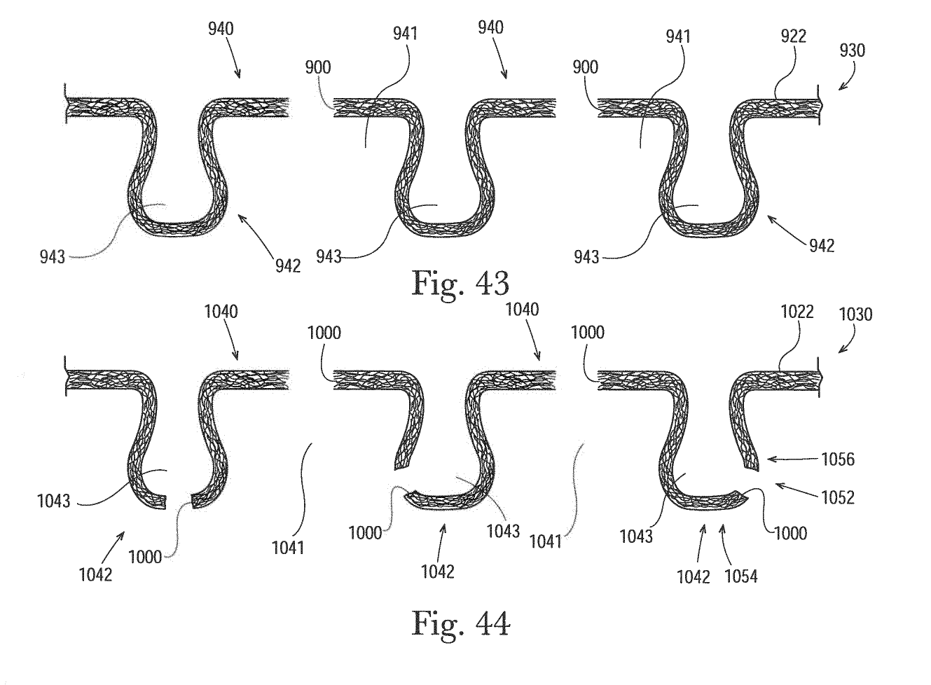

Referring to FIG. 43, a schematic illustration of a nonwoven material 930 is illustrated. The nonwoven material 930 may have one or more layers (although one is illustrated for simplicity in illustration). Apertures 900 may be formed in the generally planar first regions 940 through one or more of the layers. In some instances, if the apertures 900 are formed through all of the layers, the apertures 900 may be coincident. Unintentional tears in one or more layers may be formed in the discrete integral second regions 942, as described with respect to FIGS. 15C-15F. In some instances, it may be desirable to have the apertures 900 in only a top layer (e.g., a topsheet) and no apertures 900 in a second layer (e.g., an acquisition layer), so that BM or other bodily fluids may move through the aperture 900 and directly contact the second layer for superior absorption.

Referring to FIG. 44, a schematic illustration of a nonwoven material 1030 is illustrated. The nonwoven material 1030 may have one or more layers (although one is illustrated for simplicity in illustration). Apertures 1000 may be formed in the generally planar first regions 1040 through one or more of the layers and the discrete integral second regions 1042 through one or more layers. In some instances, if the apertures 1000 are formed through all of the layers, the apertures 1000 may be coincident. The apertures 1000 in the discrete integral second regions 1042 may be formed at least partially in the side walls 1056, at least partially in the cap 1052, or at least partially in the distal ends 1054. In a certain nonwoven material, the apertures 1000 in the various discrete integral second regions 1042 may be at least partially in the side walls 1056, at least partially in the cap 1052, or at least partially in the distal ends 1054 (or may be in all of the same). For example, a nonwoven material may have apertures 1000 in the distal ends 1052 of some discrete integral second regions 1042 and apertures 1000 in side walls of other discrete integral second regions 1042. The apertures 1000 in generally planar first region 1040 of the nonwoven material 1030 may or may not be present. In some instances, it may be desirable to have the apertures 1000 in the generally planar first region 1040 in only a top layer (e.g., a topsheet) and no apertures 1000 in a second layer (e.g., an acquisition layer), so that BM or other bodily fluids may move through the aperture 1000 and directly contact the second layer for superior absorption.

Some current two-dimensional apertured topsheets are effective at allowing BM to pass through the topsheet into the layers below. These two-dimensional apertured topsheets, however, provide very little void volume under themselves in that the generally planar topsheets are in a facing relationship with the generally planar layer below (typically at acquisition layer). Thus, BM or other bodily fluid acquisition of these two-dimensional apertured topsheets has its limits and needs to be improved. The three-dimensional nonwoven materials of the present disclosure having apertures provide this improvement in BM or other bodily fluid acquisition, while also providing reduced skin marking and softness, owing to only having small apertures present on generally planar wearer-facing surfaces 922, 1022. The nonwoven materials 930 and 1030 of FIGS. 43 and 44, provide reservoirs 941, 1041 between the discrete integral second regions 942, 1042 (if apertures are provided in the generally planar first region 940, 1040) and/or provide reservoirs 943, 1043 within the discrete integral second regions 942, 1042. These reservoirs 941, 1041, 943, 1043 provide void volume for BM or other bodily fluid retention so that such BM or other bodily fluids can be absorbed into an absorbent core positioned under the nonwoven materials or can be at least partially dewatered by the absorbent core. It is important to note the bulbous shape of the discrete integral second regions 942, 1042. This bulbous shape allows for small openings near the wearer-facing surfaces 922, 1022 in the discrete integral second regions 942, 1042, but opens to larger reservoirs 943, 1043 in the discrete integral second regions 942, 1042. With the openings near the wearer-facing surfaces 922, 1022 in the discrete integral second regions 942, 1022 being smaller (or smaller in width than areas of the discrete integral second regions 942, 1042, near the cap 952, 1052), once BM is moved into the reservoirs 943, 1043, it at least mostly remains there and is restricted in spreading. Additionally, the nonwoven materials 930 and 1030 may act to wipe BM, or other bodily fluids off of the skin of the wearer, during wearer movement. Further, the nonwoven materials 930 and 1030 provide high surface areas and contact with the skin, to entangle BM, or other bodily fluids, and at least reduce BM, or other bodily fluids from sticking in the skin.

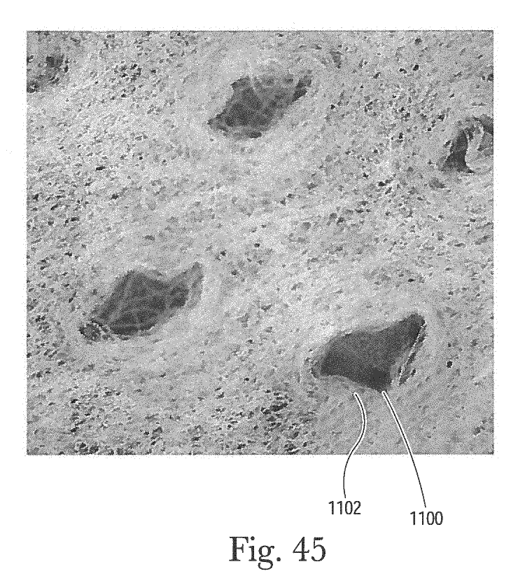

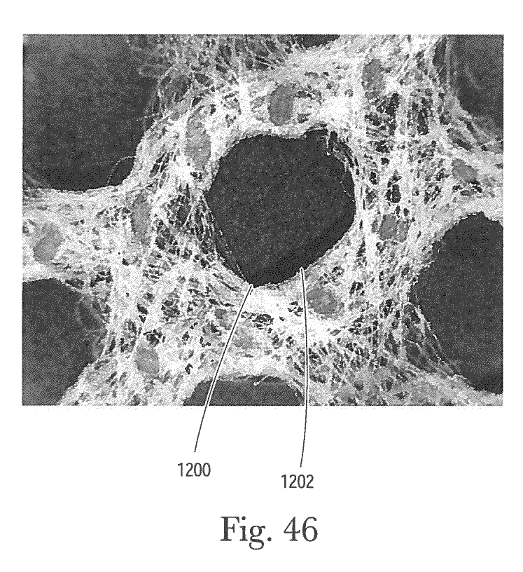

The apertures in the nonwoven materials described herein may be formed using any suitable aperturing process, such as pin aperturing, water-jet aperturing, laser aperturing, overbonding and ring rolling aperturing, cutting, and/or hot air aperturing, for example. Referring to FIG. 45, some of these types of aperturing forms a densified region 1102 around an aperture 1100, such as pin aperturing process. The densified region 1102 is caused by the pin pushing fibers outwardly from the pins and the fibers aligning and packaging together to form a densified ring of fibers around the apertures. Referring to FIG. 46, other types of aperturing forms a discontinuous melt-lip 1202 around the aperture 1200, such as overbonding and ring rolling. Over bonding and ring rolling will be described in further detail below. The discontinuous melt-lip 1202 is formed from melted portions of overbonds after the overbonds are at least partially ruptured through a ring-rolling process.

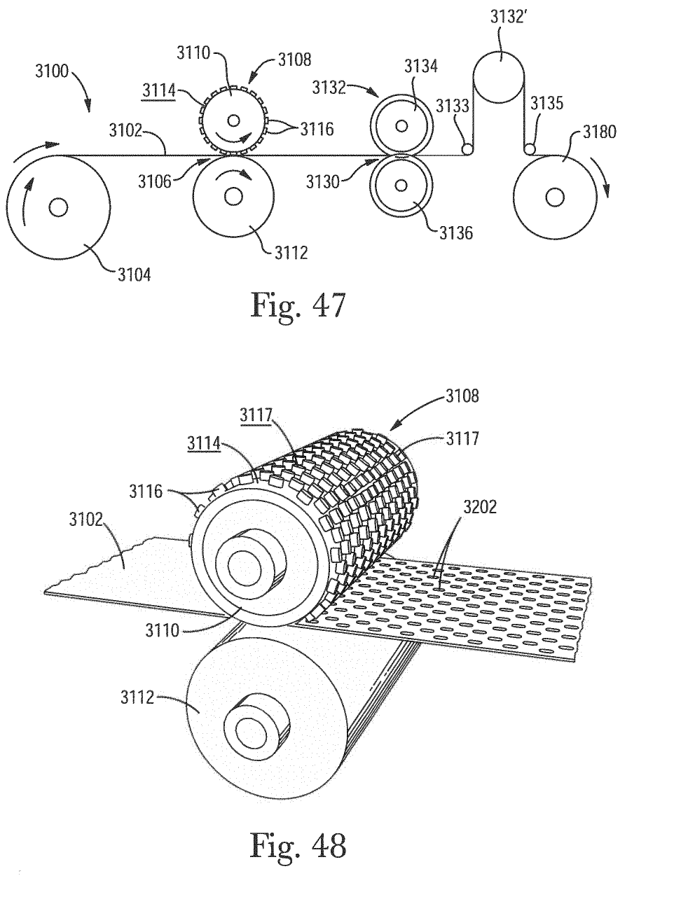

Referring to FIG. 47 there is schematically illustrated at 3100 one process for forming example apertured nonwoven materials of the present disclosure. The process may be used to aperture one nonwoven material or two or more nonwoven materials at the same time. The process may be used to aperture one nonwoven material, which may then be joined with a second apertured, or non-apertured material. If two or more layers of nonwoven material are apertured together, the apertures may be registered. If one layer of nonwoven material is apertured and then joined with another nonwoven material that has been separately apertured, the apertures may be unregistered, or at least partially unregistered.

First, a precursor material 3102 is supplied as the starting material. The precursor material 3102 may be supplied as discrete webs, e.g. sheets, patches, etc. of material for batch processing. For commercial processing, however, the precursor material 3102 may be supplied as roll stock, and, as such it can be considered as having a finite width and an infinite length. In this context, the length is measured in the machine direction (MD). Likewise, the width is measured in the cross machine direction (CD).

The precursor material 3102 may be one or more nonwoven materials (same or different), one or more films (same or different), a combination of one or more nonwoven materials and one or more films, or any other suitable materials or combinations thereof. In an instance, the precursor material 3102 may comprise a topsheet, an acquisition layer, a tissue layer, a distribution layer, and/or other layer or layers of an absorbent article, for example. The precursor material 3102 may be purchased from a supplier and shipped to where the nonwoven materials of the present disclosure are being formed or the precursor material 3102 formed at the same location as where the nonwoven materials of the present disclosure are being produced.

The precursor material 3102 may be extensible or non-elastic.

The precursor material 3102 may comprise or be made of mono-component, bi-component, multi-constituent blends, or multi-component fibers comprising one or more thermoplastic polymers. In an example, the bicomponent fibers of the present disclosure may be formed of a polypropylene core and a polyethylene sheath, a polypropylene core and polypropylene sheath, or a polyethylene core and a polyethylene sheath. Further details regarding bi-component or multi-component fibers and methods of making the same may be found in U.S. Patent Application Publ. No. 2009/0104831, published on Apr. 23, 2009, U.S. Pat. No. 8,226,625, issued on Jul. 24, 2012, U.S. Pat. No. 8,231,595, issued on Jul. 31, 2012, U.S. Pat. No. 8,388,594, issued on Mar. 5, 2013, and U.S. Pat. No. 8,226,626, issued on Jul. 24, 2012. The various fibers may be sheath/core, side-by-side, islands in the sea, or other known configurations of fibers. The fibers may be round, hollow, or shaped, such as trilobal, ribbon, capillary channel fibers (e.g., 4DG). The fibers may comprise microfibers or nanofibers.

The precursor material 3102 may be unwound from a supply roll 3104 and travel in a direction indicated by the arrow associated therewith as the supply roll 3104 rotates in the direction indicated by the arrow associated therewith. The precursor material 3102 may pass through a nip 3106 of a weakening roller (or overbonding) arrangement 3108 formed by rollers 3110 and 3112, thereby forming a weakened precursor material. The weakened precursor material 3102 may have a pattern of overbonds, or densified and weakened areas, after passing through the nip 3106. At least some of, or all of, these overbonds may be used to form apertures in the precursor material 3102. Therefore, the overbonds may correlate generally to the patterns of apertures created in the precursor material 3102.

Referring to FIG. 48, the precursor material weakening roller arrangement 3108 may comprises a patterned calendar roller 3110 and a smooth anvil roller 3112. One or both of the patterned calendar roller 3110 and the smooth anvil roller 3112 may be heated and the pressure between the two rollers may be adjusted by known techniques to provide the desired temperature, if any, and pressure to concurrently weaken and melt-stabilize (i.e., overbond) the precursor material 3102 at a plurality of locations 3202. The temperature of the calendar roller 3110 (or portions thereof) and/or the smooth anvil roller 3112 (or portions thereof) may be ambient temperature or may be in the range of about 100° C. to about 300° C., about 100° C. to about 250° C., about 100° C. to about 200° C., or about 100° C. to about 150° C., specifically reciting all 0.5° C. increments within the specified ranges and all ranges formed therein or thereby. The pressure between the calendar roller 3110 and the smooth anvil roller 3112 may be in the range of about 2,000 pli (pounds per linear inch) to about 10,000 pli, about 3,000 pli to about 8,000 pli, or about 4,500 to about 6,500 pli, specifically reciting all 0.1 pli increments within the specified ranges and all ranges formed therein or thereby. As will be discussed in further detail below, after the precursor material 3102 passes through the weakening roller arrangement 3108, the precursor material 3102 may be stretched in the CD, or generally in the CD, by a cross directional tensioning force to at least partially, or fully, rupture the plurality of weakened, melt stabilized locations 3202, thereby creating a plurality of at least partially formed apertures in the precursor material 3102 coincident with the plurality of weakened, melt stabilized locations 3202.

The patterned calendar roller 3110 may be configured to have a cylindrical surface 3114, and a plurality of protuberances or pattern elements 3116 which extend outwardly from the cylindrical surface 3114. The pattern elements 3116 are illustrated as a simplified example of a pattern of a patterned calendar roller 3110, but other patterned calendar rollers with other patterns may also be used. The protuberances 3116 may be disposed in a predetermined pattern with each of the protuberances 3116 being configured and disposed to precipitate a weakened, melt-stabilized location in the precursor material 3102 to affect a predetermined pattern of weakened, melt-stabilized locations 3202 in the precursor material 3102. The protuberances 3116 may have a one-to-one correspondence to the pattern of melt stabilized locations in the precursor material 3102. As shown in FIG. 48, the patterned calendar roller 3110 may have a repeating pattern of the protuberances 3116 which extend about the entire circumference of surface 3114. Alternatively, the protuberances 3116 may extend around a portion, or portions of the circumference of the surface 3114. Also, a single patterned calendar roller may have a plurality of patterns in various zones (i.e., first zone, first pattern, second zone, second pattern). The protuberances 3116 may have a cross-directional width in the range of about 0.1 mm to about 10 mm, about 0.1 mm to about 5 mm, about 0.1 mm to about 3 mm, about 0.15 mm to about 2 mm, about 0.15 mm to about 1.5 mm, about 0.1 mm to about 1 mm, about 0.1 mm to about 0.5 mm, or about 0.2 to about 0.5 mm, specifically reciting all 0.05 mm increments within the specified ranges and all ranges formed therein or thereby. The protuberances 3116 may have an aspect ratio in the range of about 10:1, about 9:1, about 8:1, about 7:1, about 6:1, about 5:1, about 4:1, about 3:1, about 2:1, about 1.5:1, or about 1.1:1, for example. Other aspect ratios of the protuberances 3116 are also within the scope of the present disclosure. The protuberances 3116, in some forms, may be angled, relative to the machine direction on either side. Spacing between adjacent protuberances 3116 in any direction may be greater than about 0.5 mm, greater than about 0.6 mm, greater than about 0.7 mm, greater than about 0.8 mm, greater than about 0.9 mm, greater than about 1 mm, greater than about 1.1 mm, greater than about 1.2 mm, greater than about 1.3 mm, greater than about 1.4 mm, greater than about 1.5 mm, greater than about 2 mm, greater than about 3 mm, or may be in the range of about 0.7 mm to about 20 mm, or about 0.8 to about 15 mm, specifically reciting all 0.1 mm increments within the specified ranges and all ranges formed therein or thereby.

The protuberances 3116 may extend radially outwardly from the surface 3114 and have distal end surfaces 3117. The anvil roller 3112 may be a smooth surfaced, circular cylinder of steel, rubber or other material. The anvil roller 3112 and the patterned calendar roller 3110 may be switched in position (i.e., anvil on top) and achieve the same result.

From the weakening roller arrangement 3108, the material 3102 may then be passed through a nip 3130 formed by an incremental stretching system 3132 employing opposed pressure applicators having three-dimensional surfaces which at least to a degree may be complementary to one another. The incremental stretching system 3132 is optional. Instead, the material 3102 may be instead sent through the process of FIG. 21 to break the overbonds and strain the material 3102.

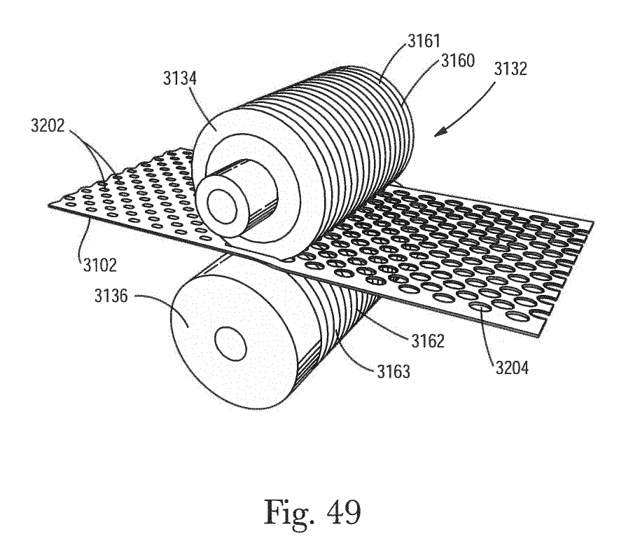

Referring now to FIG. 49, there is shown a fragmentary enlarged view of the incremental stretching system 3132 comprising two incremental stretching rollers 3134 and 3136. The incremental stretching roller 3134 may comprise a plurality of teeth 3160 and corresponding grooves 3161 which may extend about the entire circumference of roller 3134. The incremental stretching roller 3136 may comprise a plurality of teeth 3162 and a plurality of corresponding grooves 3163. The teeth 3160 on the roller 3134 may intermesh with or engage the grooves 3163 on the roller 3136 while the teeth 3162 on the roller 3136 may intermesh with or engage the grooves 3161 on the roller 3134. The spacing and/or pitch of the teeth 3162 and/or the grooves 3163 may match the pitch and/or spacing of the plurality of weakened, melt stabilized locations 3202 in the precursor material 3102 or may be smaller or larger. As the precursor material 3102 having weakened, melt-stabilized locations 3202 passes through the incremental stretching system 3132 the precursor material 3102 may be subjected to tensioning in the CD causing the material 3102 to be extended (or activated) in the CD, or generally in the CD. Additionally the material 3102 may be tensioned in the MD, or generally in the MD. The CD tensioning force placed on the material 3102 may be adjusted such that it causes the weakened, melt-stabilized locations 3202 to at least partially, or fully, rupture thereby creating a plurality of partially formed, or formed apertures 3204 coincident with the weakened melt-stabilized locations 3202 in the material 3102. However, the bonds of the material 3102 (in the non-overbonded areas) are strong enough such that they do not rupture during tensioning, thereby maintaining the material 3102 in a coherent condition even as the weakened, melt-stabilized locations rupture. However, it may be desirable to have some of the bonds rupture during tensioning.

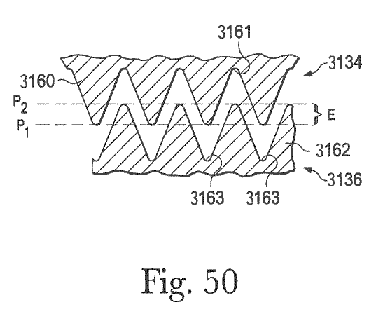

Referring to FIG. 50, a more detailed view of the teeth 3160 and 3162 and the grooves 3161 and 3163 on the rollers 3134 and 3136 is illustrated. The term “pitch” refers to the distance between the apexes of adjacent teeth. The pitch may be between about 0.02 inches to about 0.30 inches (about 0.51 mm to about 7.62 mm) or may be between about 0.05 inches and about 0.15 inches (about 1.27 mm to about 3.81 mm), specifically reciting all 0.001 inch increments within the above-specified ranges and all ranges formed therein or thereby. The height (or depth) of the teeth is measured from the base of the tooth to the apex of the tooth, and may or may not be equal for all teeth. The height of the teeth may be between about 0.010 inches (about 0.254 mm) and about 0.90 inches (about 22.9 mm) or may be between about 0.025 inches (about 0.635 mm) and about 0.50 inches (about 12.7 mm), specifically reciting all 0.01 inch increments within the above-specified ranges and all ranges formed therein or thereby. The teeth 3160 in one roll may be offset by about one-half of the pitch from the teeth 3162 in the other roll, such that the teeth of one roll (e.g., teeth 160) mesh in the valley (e.g., groove 163) between teeth in the mating roll. The offset permits intermeshing of the two rolls when the rolls are “engaged” or in an intermeshing, operative position relative to one another. The teeth of the respective rolls may only be partially intermeshing in some instances. The degree to which the teeth on the opposing rolls intermesh is referred to herein as the “depth of engagement” or “DOE” of the teeth. The DOE may be constant or not constant. As shown in FIG. 50, the DOE, indicated as “E”, is the distance between a position designated by plane P1 where the apexes of the teeth on the respective rolls are in the same plane (0% engagement) to a position designated by plane P2 where the apexes of the teeth of one roll extend inward beyond the plane P1 toward the groove on the opposing roll. The optimum or effective DOE for particular laminate webs may be dependent upon the height and the pitch of the teeth and/or the structure of the material. Some example DOEs may in the range of about 0.01 inches to about 0.5 inches, about 0.03 inches to about 0.2 inches, about 0.04 inches to about 0.08 inches, about 0.05 inches, or about 0.06 inches, specifically reciting all 0.001 inch increments within the above-specified ranges and all ranges formed therein or thereby.

As the material 3102 having the weakened, melt-stabilized locations 3202 passes through the incremental web stretching apparatus 3132, the material 3102 may be subjected to tensioning in the cross machine direction, or substantially in the cross machine direction, thereby causing the nonwoven web 3102 to be extended in the cross machine direction. The tensioning force placed on the material 3102 may be adjusted by varying the pitch, DOE, or teeth size, such that the incremental stretching is sufficient to cause the weakened, melt-stabilized locations 3202 to at least partially, or fully rupture, thereby creating, or at least partially creating, a plurality of apertures 3204 coincident with the weakened, melt-stabilized locations 3202 in the material 3102.

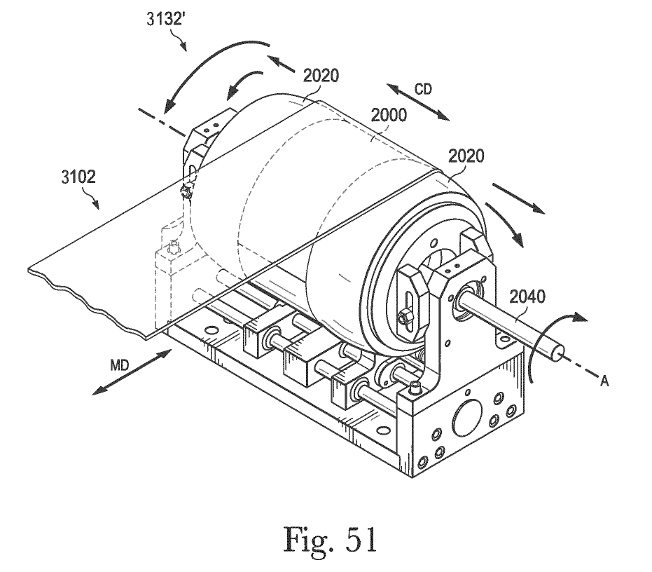

After the material 3102 passes through the incremental web stretching apparatus 3132, the web 3102 may be advanced to and at least partially around a cross machine directional tensioning apparatus 3132′ (see e.g., FIGS. 47 and 51). The cross machine directional tensioning apparatus 3132′ may be offset from the main processing line by running the web partially around two idlers 3133 and 3135 or stationary bars, for example. In other instances, the cross machine tensioning apparatus 3132′ may be positioned in line with the main processing line. The cross machine directional tensioning apparatus 3132′ may comprise a roll that comprises at least one outer longitudinal portion that expands along a longitudinal axis, A, of the roll, relative to a middle portion of the roll, to stretch and/or expand the material 3102 in the cross machine direction. Instead of or in addition to expanding along the longitudinal axis, A, of the roll, the outer longitudinal portion may be angled relative to the longitudinal axis, A, of the roll in a direction away from the material 3102 being advanced over the roll to stretch the material 3102 in the cross machine direction or generally in the cross machine direction. In an instance, the roll may comprise two outer longitudinal portions that each may expand in opposite directions generally along the longitudinal axis, A, of the roll. The two outer portions may both be angled downwards in a direction away from the material 3102 being advanced over the roll. This movement or positioning of the outer longitudinal portions of the roll may allow for generally cross machine directional tensioning of the material 3102, which causes the plurality of weakened locations 3202 to rupture and/or be further defined or formed into apertures 3204.

The outer longitudinal portions of the roll may comprise vacuum, a low tack adhesive, a high coefficient of friction material or surface, such as rubber, and/or other mechanisms and/or materials to hold the material 3102 to the outer lateral portions of the roll during movement of the outer longitudinal portion or portions relative to the middle portion of the roll. The vacuum, low tack adhesive, high coefficient of friction material or surface, and/or other mechanisms and/or materials may prevent, or at least inhibit, the held portions of the material 3102 from slipping relative to the longitudinal axis, A, of the roll during stretching of the outer lateral portions of the material in the cross machine direction or generally in the cross machine direction.

FIG. 51 is a top perspective view of the example cross machine directional tensioning apparatus 3132′. The cross machine directional tensioning apparatus 3132′ may comprise a roll comprising a middle portion 2000 and two outer longitudinal portions 2020 situated on either end of the middle portion 2000. The roll may rotate about its longitudinal axis, A, on a drive shaft 2040. The roll may rotate relative to the drive shaft 2040 or in unison with the drive shaft 2040, as will be recognized by those of skill in the art. The material 3102 may be advanced over the entire cross machine directional width of the middle portion 2000 and at least portions of the cross machine directional widths of the outer longitudinal portions 2020. The material 3102 may be advanced over at least about 5% up to about 80% of the circumference of the roll so that the cross machine directional stretching may be performed.

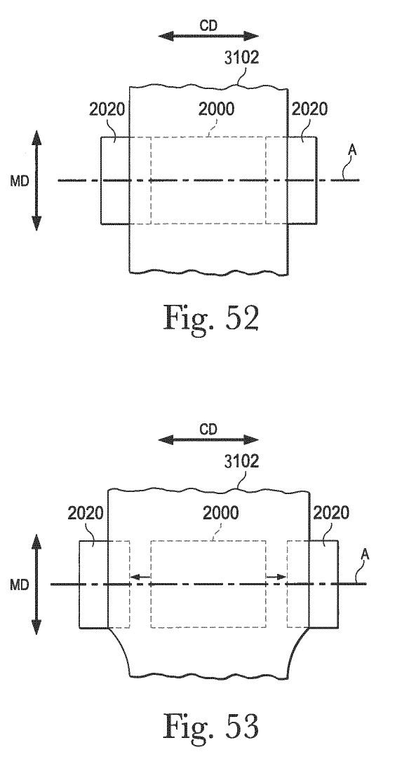

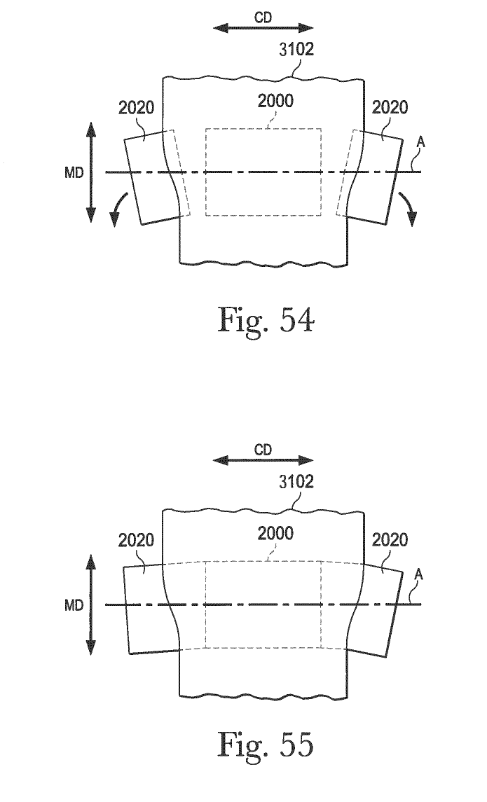

FIG. 52 is a schematic representation of a front view of an example cross machine directional tensioning apparatus with outer longitudinal portions 2020 in an unexpanded or non-angled position relative to the middle portion 2000. FIG. 53 is a schematic representation of a front view of the cross machine directional tensioning apparatus of FIG. 52 with the outer longitudinal portions 2020 in a longitudinally expanded position relative to the middle portion 2000. FIG. 54 is a schematic representation of a front view of the cross machine directional tensioning apparatus of FIG. 52 with the outer longitudinal portions 2020 in an angled and expanded position relative to the middle portion 2000. In regard to FIG. 54, the outer longitudinal portions 2020 may merely move or slide in a direction generally perpendicular to the machine direction of the material passing over the roll to apply the cross machine directional tensioning force to the material 3102. FIG. 55 is a schematic representation of a front view of a cross machine directional tensioning apparatus with the outer longitudinal portions 2020 fixed in an angled position relative to the middle portion 2000 to apply the cross machine directional tensioning force to the material 3102. In such a form, the middle portion 2000 and each of the outer longitudinal portions 2020 may comprise a separate roll.

Regardless of whether one or both of the outer longitudinal portions 2020 is moved, slid, rotated, fixed, and/or expanded relative to the middle portion 2000, this relative motion or positioning between the outer longitudinal portions 2020 and the middle portion 2000 stretches the materials 3102 in a cross machine direction to further rupture or further define the weakened locations 2020 in the material 3102 and create, or further form, a plurality the apertures 2040 the material 3102. The cross machine directional tensioning force applied by the cross machine directional tensioning apparatus 3132′ may be, for example, 10-25 grams or 15 grams. In an instance, the cross machine directional tensioning apparatus may be similar to, or the same as, the incremental stretching apparatus 3132 to apply the cross machine directional tensioning force. In still other instances, any suitable cross machine directional tensioning apparatus may be used to apply the cross machine directional tensioning force to the material 3102.

If desired, the incremental stretching step or the cross machine directional stretching step described herein may be performed at elevated temperatures. For example, the material 3102 and/or the rolls may be heated. Utilizing heat in the stretching step may serve to soften the material, and may aid in extending the fibers without breaking.

Referring again to FIG. 47, the material 3102 may be taken up on wind-up roll 3180 and stored. Alternatively, the material 3102 may be fed directly to a production line where it is used to form a portion of an absorbent article or other consumer product.

It is important to note that the overbonding step illustrated in FIGS. 47 and 48 could be performed by the material supplier and then the material may be shipped to a consumer product manufacturer to perform step 3132. In fact, the overbonding step may be used in the nonwoven production process to form overbonds, which may be in addition to, or in lieu of, primary bonds formed in the nonwoven production process. Alternatively, the material supplier may fully perform the steps illustrated in FIG. 47 and then the material may be shipped to the consumer product manufacturer. The consumer product manufacturer may also perform all of the steps in FIG. 47 after obtaining a nonwoven material from a nonwoven material manufacturer.

One of ordinary skill in the art will recognize that it may be advantageous to submit the material 3102 to multiple incremental stretching processes depending on various desired characteristics of the finished product. Both the first and any additional incremental stretching may either be done on-line or off-line. Furthermore, one of ordinary skill will recognize that the incremental stretching may be done either over the entire area of the material or only in certain regions of the material depending on the final desired characteristics.

The overbonding and ring rolling process described with respect to FIGS. 47-55 or other aperturing processes, such as pin aperturing, may be used to aperture one layer, or multiple layers of a nonwoven material. As a first example, a topsheet or a first layer may be apertured and then may be joined to, or brought together with, an acquisition layer or a second layer. As a second example, an acquisition layer or a second layer may be apertured and then joined to, or brought together with, a topsheet or a second layer. As a third example, both layers may be apertured together or apertured separately and then brought together.

A plurality of different methods may be used to create a three-dimensional nonwoven material with apertures. In an instance, a first layer (e.g., a topsheet, acquisition layer, or other layer) may be overbonded (e.g., FIG. 48), brought together with one or more second non-overbonded layers (e.g., acquisition layer, topsheet, or other layer), and then run through the process of FIG. 21 to create a three-dimensional structure and join the first and second layers together. The three-dimensional material may then be run through at least some of the processes of FIGS. 49 and 51-55 to rupture the overbonds in the first layer to form apertures in the first layer. At least some of the apertures will not be registered with the generally planar first regions or the discrete integral second regions in the three-dimensional nonwoven material.

In other instances, a first layer (e.g., a topsheet, acquisition layer, or other layer may be overbonded (e.g., FIG. 48), brought together with one or more second non-overbonded layers (e.g., acquisition layer, topsheet, or other layers), and then run through the process of FIG. 21 to rupture the overbonds. An additional cross-directional spreading step may be used (e.g., FIGS. 51-55) to further rupture the overbonds.

In an instance, a first layer (e.g., a topsheet, acquisition layer, other layer) may be pin apertured or otherwise apertured (either at a supplier or upstream in the process), then may be brought together with one or more second non-apertured layers (e.g., acquisition layer, topsheet, or other layer), and then may be run through the process of FIG. 21 to create a three-dimensional structure and join the first and second layers together. At least some of the apertures will not be registered with the generally planar first regions or the discrete integral second regions in the three-dimensional nonwoven material (see e.g., FIG. 70). Instead, apertures will likely be formed in the generally planar first regions and in the discrete integral second regions. The areas of the apertures in the generally planar first regions of the first layer may stay about the same and the areas of the apertures in the discrete integral second regions may get smaller or larger after the process of FIG. 21 compared to the areas of the original apertures in the first layer. This may also apply when two or more layers are pre-apertured and then run through the process of FIG. 21. The aspect ratios of the apertures in the generally planar first regions of the first layer may stay about the same and the aspect ratios of the apertures in the discrete integral second regions may become smaller or larger after the process of FIG. 21 compared to the aspect ratios of the original apertures in the first layer. This may also apply when two or more layers are pre-apertured and then run through the process of FIG. 21.

In an instance, a first layer (e.g., a topsheet, acquisition layer, or other layer) may be overbonded (e.g., FIG. 48) and run through at least some of the processes of FIGS. 49 and 51-55 to rupture the overbonds in the first layer and form apertures in the first layer. The apertured first layer may then be brought together with one or more second non-overbonded layers (e.g., acquisition layer, topsheet, or other layer), and then run through the process of FIG. 21 to create a three-dimensional structure and join the first and second layers together. At least some of the apertures will not be registered with the generally planar first regions or the discrete integral second regions in the three-dimensional nonwoven material.

In an instance, a two or more layer laminate (e.g., a topsheet and an acquisition layer, or two other layers) may be overbonded (e.g., FIG. 48) and then run through the process of FIG. 21 to create a three-dimensional material and further join the first and second layers together. The three-dimensional material may then be run through at least some of the processes of FIGS. 49 and 51-55 to rupture the overbonds to form apertures coincident apertures in the laminate. At least some of the apertures will not be registered with the generally planar first regions or the discrete integral second regions of the three-dimensional material.

In an instance, a two or more layer laminate (e.g., a topsheet and an acquisition layer, or two other layers) may be overbonded (e.g., FIG. 48) and then run through the process of FIG. 21 to create a three-dimensional material and further join the first and second layers together and rupture the overbonds. An additional cross-directional spreading step may be used (e.g., FIGS. 51-55) to further rupture the overbonds. At least some of the apertures will not be registered with the generally planar first regions or the discrete integral second regions of the three-dimensional material.

In an instance, a two or more layer laminate (e.g., a topsheet and an acquisition layer, or two other layers) may be brought together and overbonded (e.g., FIG. 48) and then run through the process of FIGS. 49 and 51-55 to create coincident apertures in the laminate. The apertured laminate may then be run through the process of FIG. 21 to create a three-dimensional material and further join the first and second layers together. At least some of the apertures will not be registered with the generally planar first regions or the discrete integral second regions of the three-dimensional nonwoven material.

In an instance, a two or more layer laminate (e.g., a topsheet and an acquisition layer, or two or more other layers) may be brought together and pin apertured or otherwise apertured, and then run through the process of FIG. 21 to create a three-dimensional material and further join the two or more layers together. At least some of the apertures will not be registered with the generally planar first regions or the discrete integral second regions of the three-dimensional nonwoven material.

In an instance, a two or more layer laminate (e.g., a topsheet and an acquisition layer, or two or more other layers) may be separately pre-apertured (using any suitable processes), then brought together, and then run through the process of FIG. 21 to create a three-dimensional material and join the two or more layers together. At least some of the apertures will not be registered with the generally planar first regions or the discrete integral second regions of the three-dimensional nonwoven material.

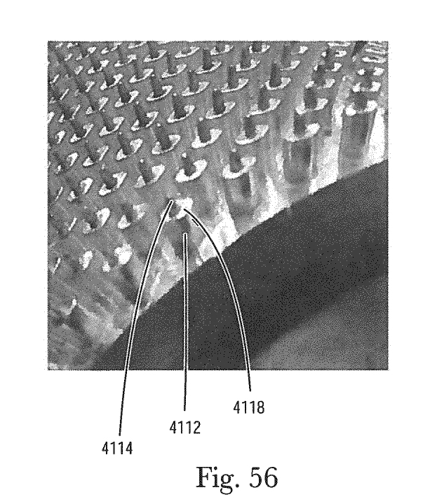

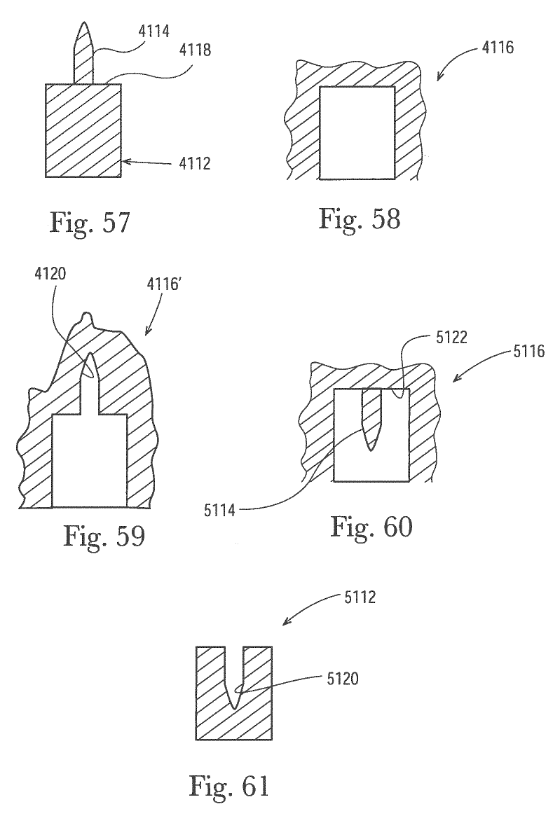

In some forms, apertures and the three-dimensional structures may be created in nonwoven materials using a single process. Referring to FIGS. 21 and 56, the discrete, spaced apart male forming elements 112 of FIG. 21, in other forms, may comprise discrete, spaced apart male forming elements 4112 comprising pins 4114 extending outwardly relative to a top 4118 of the male forming elements 4112. The pins 4114 may be used to form apertures in materials being run through the first and second forming members 102 and 104 (see FIG. 21). An example cross-sectional illustration of one of the male forming elements 4112 comprising the pin 4114 is disclosed in FIG. 57. The pins 4114 may be joined to or formed with the male forming elements 4112 and may comprise the same materials or different materials. The male forming element 4112 may engage a female forming element 4116 having the cross-sectional shape illustrated in FIG. 58. In such an instance, the female forming element 4116 may be elongated enough to receive at least part of the male forming element 4116 and the pin 4114. In other instances, the male forming element 4112 may engage a female forming element 4116′ having the cross-sectional shape illustrated in the FIG. 59. The female forming element 4116′ may define a pin-receiving cavity 4120. The pin-receiving cavity 4120 may or may not match the shape of the pin 4114, but may be, for example, an elongated cylinder. FIG. 60 is a cross sectional illustration of a female forming element 5116. The female forming element 5116 may comprise a pin 5114 extending outwardly from a bottom surface 5122 thereof. The pin 5114 may be formed with or joined to the female forming element 5116 and make comprise the same materials as the female forming element 5116 or different materials. FIG. 61 is a cross-sectional illustration of a male forming element 5112 that may be used with the female forming element 5116 of FIG. 60. Referring to FIG. 61, the male forming element 5112 may define a pin-receiving cavity 5120. The pin-receiving cavity 5120 may be configured to at least partially receive the pin 5114.

By using either male or female forming elements having pins, the pins may form apertures in a substrate passing through the first and second forming members 102 and 104 (see FIG. 21). In addition to the apertures being created, the three-dimensional structure may be formed at the same time, or substantially at the same time. In such an instance, the apertures are formed in the discrete integral second regions.

In other instances, pins may be located intermediate male forming elements on the first forming member 102 to create apertures in portions of the generally planar first region. Pin receiving-cavities may be formed on the second forming member 104 to at least partially receive the pins. In other instances, pins may be located intermediate female forming elements on the second forming roll 104 to create apertures in portions of the generally planar first region. Pin receiving-cavities may be formed on the first forming member 102 to at least partially receive the pins. Either of the first or second forming members 102, 104 may be heated to enable better aperture formation. Using the apparatuses described in this paragraph, apertures may be formed in portions of the generally planar first region. Apertures may also be formed the discrete integral second regions, as described in the preceding paragraph.

### V. Strip Forms

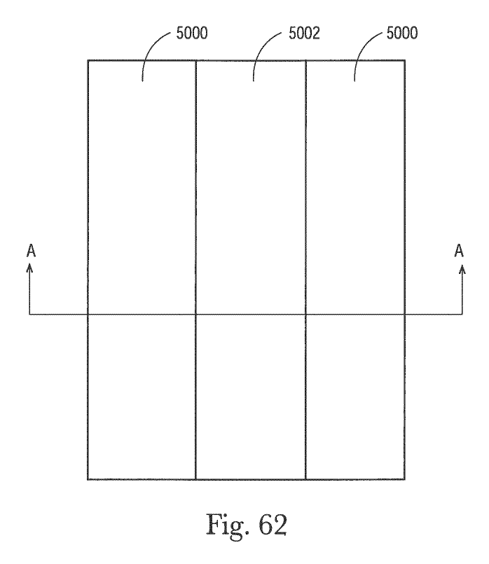

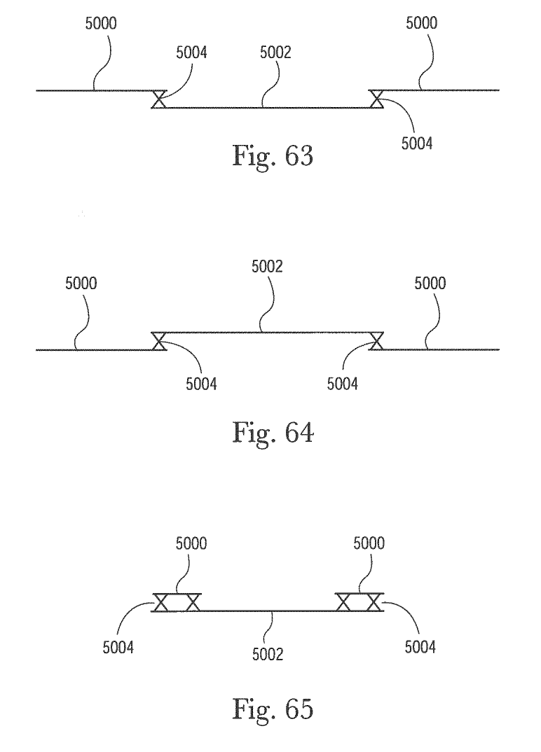

In an instance, whether the nonwoven material has apertures or not, in an absorbent article context, portions of a nonwoven acquisition material may form a portion of a wearer-facing surface. FIG. 62 is a plan view of an example topsheet material and acquisition material configuration for an absorbent article, wearer-facing surface facing the viewer. FIGS. 63-65 are example cross-sectional views taken about line A-A of FIG. 62. Referring to FIG. 62, a topsheet material 5000 may be formed in two strips, while an acquisition material 5002 may be formed of a single strip. Although not illustrated in FIGS. 62-65, the topsheet material 5000 and/or the acquisition material 5002 may have the generally planar first region (e.g., 40) and the discrete integral second regions (e.g., 42) disclosed herein. In some instances, at least the acquisition material 5002 may have the generally planar first region and the discrete integral second regions. Either of the generally planar first regions or the discrete integral second regions, or both of them, (in either the topsheet 5000 and/or the acquisition material 5002) may have the apertures described herein. The apertures may be defined through portions of at least some of the plurality of discrete integral second regions or through portions of the generally planar first region. The apertures may be formed in a predetermined, intentional pattern, or in random patterns.

An absorbent article, such as a diaper or a sanitary napkin, may comprise an absorbent core, a backsheet, a first end edge, a second end edge, a first side edge, a second side edge, and a three-piece topsheet forming at least a portion of a wearer-facing surface. The three-piece topsheet may comprise a first material (or topsheet material) positioned proximate to the first side edge and extending at least partially between the first end edge and the second end edge, a second material (or topsheet material) positioned proximate to the second side edge and extending at least partially between the first end edge and the second end edge, and a third material (or acquisition material) positioned intermediate the first material and the second material and extending at least partially between the first end edge and the second end edge. The first and second materials (e.g., 5000) may comprise the same material, which may be one or more generally planar nonwoven materials. In some instances, the first and second materials 500 may be free of the plurality of discrete integral second regions, although they may be embossed, for example. The third material (e.g., 5002) may comprise a nonwoven or other acquisition material. In some instances, none of the first, second, and third materials may extend from the first side edge to the second side edge of the absorbent article. The first and second materials may have the same or substantially the same basis weights while the nonwoven acquisition material may have a different basis weight. The basis weight of the first and second material may be lower than the basis weight of the nonwoven acquisition material. The basis weight of the first and second materials may be in the range of about 5 gsm to about 25 gsm, or about 10 gsm to about 20 gsm, or about 15 gsm, for example, and the basis weight of the third material may be in the range of about 15 gsm to about 100 gsm, for example. The first and second materials may generally be much cheaper materials than the third material, thereby allowing absorbent article manufacturers to use less of the more expensive third material and save significant costs.

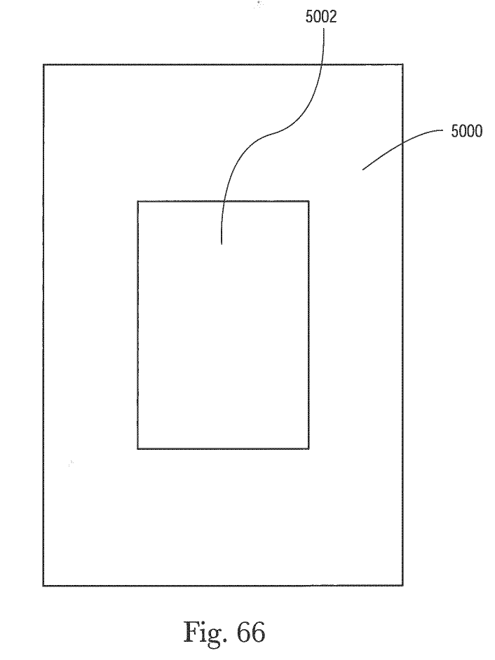

Referring to FIG. 63, portions of the acquisition material 5002 may be positioned under portions of the two topsheet materials 5000. Bonds 5004 may exist between side edge portions of the acquisition material 5002 and side edge portions of the topsheet materials 5000. The bonds 5004 may comprise ultrasonic bonds, adhesive bonds, and/or mechanical bonds, for example. Referring to FIG. 64, portions of the acquisition material 5002 may be positioned over portions of the two topsheet materials 5000. Bonds 5004 (as described above) may exist between side edge portions of the acquisition material 5002 and side edge portions of the topsheet materials 5000. The topsheet materials 5000 and the acquisition material 5002 may have any suitable overlap to allow for proper bonding. Referring to FIG. 65, side portions of the acquisition material 5002 may be positioned under all of the topsheet materials 5000, for example. In some instances, referring to FIG. 66, the acquisition material 5002 may be fully surrounded by the topsheet material 5000. The wearer-facing surface is facing the viewer in FIG. 66.

By providing an acquisition material as part of the wearer-facing surface (i.e., no topsheet material covering most of it, or all of it), BM and other bodily fluids may quickly be absorbed into an absorbent article, as the BM and other bodily fluids may directly contact the acquisition material and not the topsheet material, which typically has a lower permeability that the acquisition material. An additional advantage may be dryness as the acquisition material is typically higher in permeability and has less fluid retention than the topsheet material, thereby providing better dewatering of the acquisition material compared to a topsheet material. The presence of the three-dimensional texture in the acquisition material of the three-piece topsheet may reduce BM, or other bodily fluid spreading (i.e., run-off), improve in acquiring BM, or other bodily fluids, compared to them sticking to the skin, and improve in wiping BM or other bodily fluids off of the skin of a wearer, during wearer movement.

It may be desirable for the acquisition material (e.g., 5002) forming a portion of the wearer-facing surface of an absorbent article to have a low density to provide good permeability and void volume for quickly acquiring bodily fluids. The density of the acquisition material may be less than 0.05 g/cc, but greater than 0.01 g/cc or greater than 0.005 g/cc, or less than 0.03 g/cc, but greater than 0.01 g/cc or greater than 0.005 g/cc, for example. The low density of the acquisition material may lead to improved softness and a good cushiony feel. The low density may be achieved by specific fibers, such as spiral or bicomponent eccentric fibers, such as PE/PET or blending a fraction of thicker fibers. Additionally, the low density may be achieved by re-lofting the nonwoven acquisition material after unwinding it on an absorbent article manufacturing line, by the use of heat tunnels, for example. Softness of the acquisition materials may further be improved by using small denier fibers, for example fibers have a denier less than 4, but greater than 1, or less than 3, but greater than 1. Low density of the acquisition materials in combination with small denier fibers may still deliver sufficient permeability. Additionally, fiber softness may be improved by selecting for the fibers particular polymers, such as polyethylene or soft melt additives, or by coating the fibers with soft polymers. Further, the combination of hydrophobic and hydrophilic fibers may help with facilitating drainage of the bodily fluids into layers below the acquisition materials, wherein hydrophilic and hydrophobic fibers may be blended within the nonwoven acquisition material. In some instances, multilayer configurations of the nonwoven acquisition material may be desirable. Stated another way, the nonwoven acquisition material may be made of different layers where each layer may have different properties. The different properties may comprise fiber composition, fiber shape, hydrophilicity, and/or density. The layers may also have different deniers. The process illustrated in FIG. 21 may provide better capillary connectivity within the multilayer nonwoven acquisition material, especially in combination with hydrophilicity gradients (i.e., a garment-facing layer being more hydrophilic than a wearer-facing layer).

### VI. Zones

The apertures may be present in nonwoven materials (e.g., topsheet, or topsheet an acquisition layer laminate) in absorbent articles or other consumer products in patterns and/or zones. For example, a first zone of a nonwoven material may have a first pattern of apertures and a second zone of the nonwoven material may have a second pattern of apertures. The patterns may be the same or different. The first zone may be in the nonwoven material on a first side of a lateral axis of the absorbent article and the second zone may be in the nonwoven material on a second side of the lateral axis, for example. In other instances, the first zone may be a central area of the nonwoven material over at least a portion of a longitudinal axis of the absorbent article and the second zone may be an area at least partially, or fully, surrounding the central area of the nonwoven material. Any other suitable first zones and second zones in the nonwoven material area also within the scope of the present disclosure. More than two zones may also be provided. At least a third zone may have the same pattern of apertures as the first and/or second zones or a different pattern of apertures as the first and/or second zones. The various patterns of apertures may by different in size of apertures, areas of the apertures, shapes of the apertures, placement of the apertures (e.g., in the generally planar first regions or in the discrete integral second regions), and/or angle of the apertures relative to a longitudinal axis of a consumer product (e.g., an absorbent article), for example.

In some instances, apertures may be present in an entire topsheet, or most of the topsheet, and the three-dimensional texture may be present in only a zone. In other instances, apertures may be present in an entire topsheet and acquisition layer, or most of the topsheet and acquisition layer, and the three-dimensional texture may be present in only a zone. In yet other instances, the three-dimensional texture may be present in an entire topsheet, most of the topsheet, an entire topsheet and acquisition layer, or most of the entire topsheet and acquisition layer, and apertures may only be present in a zone of the topsheet or a zone of the topsheet and acquisition layer.

In some instances, the three-dimensional texture may be present in a first zone and apertures may be present in a second zone. The first and second zones may or may not overlap. If the first zone partially overlaps the second zone, only apertures may be present in a non-overlapping area of the second zone, only the three-dimensional texture may be present in a non-overlapping area of the first zone, and both apertures and the three-dimensional texture may be present in the overlapping area of the first and second zones.

In some instances, the three-dimensional texture may be in all zones and the apertures may be in all zones.

In some instances, where the nonwoven material comprises two layers (e.g., a topsheet and an acquisition layer), one or more certain portions of the two layers may not have an adhesive therebetween. By eliminating the adhesive in such one or more certain portions, after an insult of bodily exudates, the layers may at least partially separate and create a void intermediate the layers for receiving at least some of the bodily exudates. Stated another way, such one or more certain portions lacking an adhesive may create an unbonded window that essentially may create a pocket for receiving bodily exudates. Areas around the one or more certain portions may have an adhesive between them such that they remain laminated together even after a bodily exudate insult. In such contexts, the topsheet and acquisition layer may or may not be nested together in the unbonded window. In certain instances, only the topsheet or only the acquisition layer may have the three-dimensional texture.

### VII. Configurations

An absorbent article may comprise the two layer nested nonwoven material described herein having the generally planar first regions and the plurality of discrete integral second regions. The first layer may form a topsheet of the absorbent article. The second layer may form an acquisition layer of the absorbent article. The absorbent article may have a central lateral axis and a central longitudinal axis. The topsheet may have a first width measured parallel to the central lateral axis. The acquisition layer may have a second width measured parallel to the central lateral axis. The first width may be larger than the second width.

An absorbent article may comprise the two layer nested nonwoven material described herein having the generally planar first regions and the plurality of discrete integral second regions. The first layer may form a topsheet of the absorbent article. The second layer may form an acquisition layer of the absorbent article. The absorbent article may have a central lateral axis and a central longitudinal axis. The topsheet may have a first length measured parallel to the central longitudinal axis. The acquisition layer may have a second length measured parallel to the central longitudinal axis. The first length may be larger than the second length.

### VIII. Three-Dimensional Projections with Apertures Only in the Topsheet

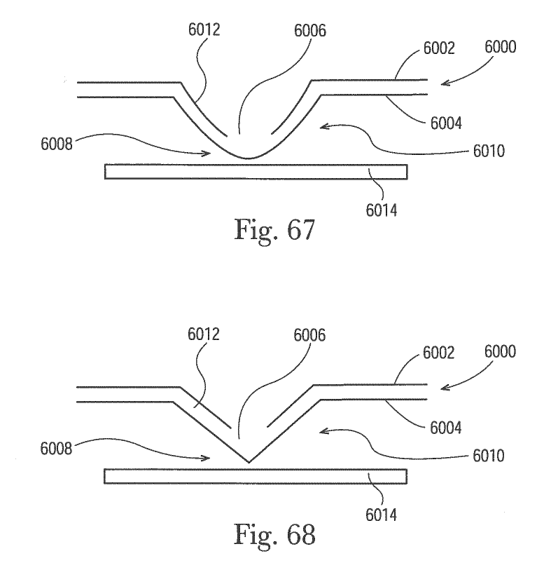

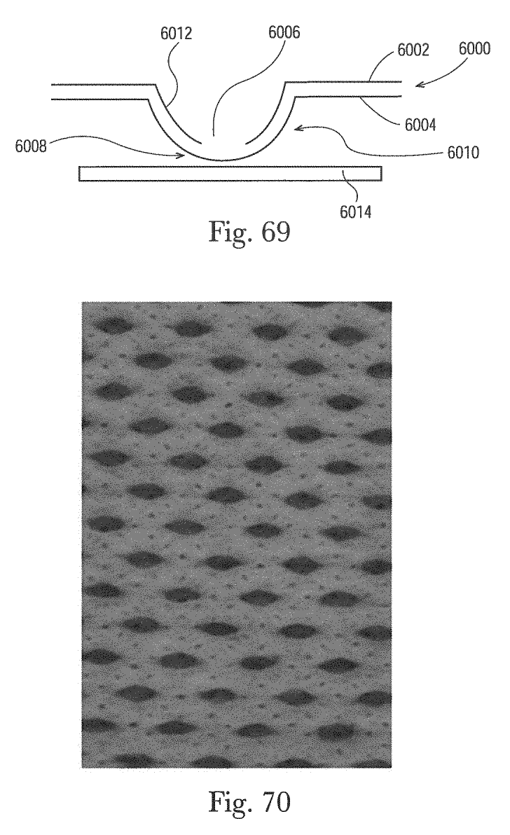

FIGS. 67-69 are cross-sectional illustrations of examples of portions of nested laminates 6000 of topsheets 6002 and acquisition layers 6004, with apertures 6006 formed in distal ends 6008 of the three-dimensional structures 6010 only in the topsheets 6002. In some instances, the apertures 6006 may also be formed in the side walls 6012. The acquisition layer (or secondary topsheet) 6004 may be free of apertures. In an absorbent article context, the three-dimensional structures 6010 may extend towards an absorbent core 6014 or may extend away from the absorbent core.

In a form, an absorbent article may comprises a nested laminate comprising a topsheet and an acquisition layer, a backsheet, and an absorbent core positioned at least partially between the nested laminate and the backsheet. The laminate may comprise a generally planar first region and a plurality of discrete integral second regions that comprise deformations forming three-dimensional protrusions extending toward the core. At least some of the plurality of discrete integral regions may have apertures formed in areas most proximal to the absorbent core. The acquisition layer may be free of apertures.

In a form, an absorbent article may comprise a nested laminate comprising a topsheet and an acquisition layer, a backsheet, and an absorbent core positioned at least partially between the nested laminate and the backsheet. The laminate may comprise a generally planar region and a plurality of discrete three-dimensional structures extending toward the core. At least some of the plurality of discrete three-dimensional structures may have apertures formed in areas proximal to the absorbent core. The acquisition layer may be free of apertures.

### IX. Examples of Performance with Apertures

A few different topsheet/acquisition layer (TS/AQL) laminates were tested according to the Roll Test procedure below. Each of the TS/AQL laminate samples (labeled as “codes” below) was tested in such procedure in combination with a 222 gsm cross-linked cellulosic fiber layer glued to an 8 gsm SMS (Spunbond-Meltblown-Spunbond) support layer. Cross-linked cellulosic fiber layers have been used in disposable diapers as part of an acquisition/distribution system, for example, U.S. Pat. Publ. No. 2008/0312622 A1 to Hundorf. The TS/AQL laminate is placed with the AQL side facing the cellulosic fiber layer. The TS/AQL laminate is positioned on the cellulosic fiber layer such that it is centered over both a central lateral axis of the cellulosic fiber layer and a central longitudinal axis of the cellulosic fiber layer. The other side of the cellulosic fiber layer is facing the 8 gsm SMS support layer. The support layer is facing a flat board, such that the entire composite is on the flat board. The laminate is then secured on the board via lateral hooks present on the sides of the board. The TS/AQL laminate was 380 mm long and 180 mm wide and the AQL was 90 mm wide. The cellulosic fiber layer was 235 mm long and 80 mm wide and had a density of ca. 0.05 g/cm³.

The test fluid is a solution made with 0.5% by weight Carbopol, 5% by weight 1M NaOH solution, 95.4% by weight deionized water.

After the laminate is set up and secured to the board, 5+/−0.01 grams of test fluid are gently and uniformly applied via a syringe onto the topsheet in an area which is 20 mm wide (in a direction parallel to a central lateral axis of the TS/AQL laminate) and 60 mm long (in a direction parallel to a central longitudinal axis of the TS/AQL laminate). The area has 10 mm on each side of the central longitudinal axis of the TS/AQL laminate. The 60 mm length begins at end edge of the cellulosic fiber layer and continues 60 mm toward the other end edge of the cellulosic fiber layer. One minute after the application of the test fluid, a Plexiglas roll, having a diameter of ca. 100 mm, a width of ca. 95 mm, and a weight of 1100 g, is rolled one time over the test fluid without exerting extra pressure to the roll until reaching the opposite end of the TS/AQL laminate material along the central longitudinal axis. The roll is covered with a collagen layer via double sided adhesive tape, wherein the collagen layer is replaced after each replicate of the test.

The TS/AQL laminate and the cellulosic fiber layer are weighed prior to the rolling and after the rolling. The difference between the cellulosic fiber layer's weight after the rolling and the cellulosic fiber layer's weight prior to rolling represents the amount of test fluid that is absorbed into the cellulosic fiber layer (CABS). A higher value of CABS is desired as in fact it means that there is less fluid present over and within the TS/AQL laminate: as the test fluid is a proxy for runny BM of babies, in an in-use situation, this would mean less runny BM closer to the skin of the baby.

### EXAMPLES

### Comparative Example

Code 1: Pattern of FIG. 6 herein of a bicomponent 20 gsm spunbond topsheet and 65 gsm carded airthrough bonded AQL without apertures.

### Present Disclosure Examples

Code 2: Pattern of FIG. 6 of a bicomponent 20 gsm spunbond topsheet and 65 gsm carded airthrough bonded AQL with apertures at the bottom of the plurality of discrete integral second regions, apertures 1.75 mm diameter, 5.6% effective open area (created with a hole punch).
Code 3: Pattern of FIG. 6 of a bicomponent 20 gsm spunbond topsheet and 65 gsm carded airthrough bonded AQL with apertures in the generally planar first regions, apertures 1.75 mm diameter, 5.6% effective open area.
Code 4: Pattern of FIG. 6 of a bicomponent 20 gsm spunbond topsheet and 65 gsm carded airthrough bonded AQL with apertures both at bottom of the plurality of discrete integral second regions and in the generally planar first regions, apertures 1.75 mm diameter, 11.1% effective open area.

### Data

### Amount of Test Fluid Absorbed in the Cellulosic Fiber Layer (CABS)

| | | Standard | |
|---|---|---|---|
| Code | Average, g | Deviation, g | N |
| Code 1 | 0.19 | 0.09 | 3 |
| Code 2 | 0.64 | 0.07 | 3 |
| Code 3 | 1.20 | 0.11 | 3 |
| Code 4 | 1.63 | 0.03 | 2 |

Where N is the number of replicates.

As can be seen, all of the codes containing apertures (Codes 2, 3, and 4) absorbed more fluid into the cellulosic fiber layer than the non-apertured code (Code 1). Code 4 absorbed the most fluid into the cellulosic fiber layer and Code 4 has apertures at bottom of the plurality of discrete integral second regions and in the generally planar first regions. Thus, apertures in the three-dimensional TS/AQL laminates of the present disclosure perform better in absorbent articles than three-dimensional TS/AQL laminates without apertures.

### X. Examples of Aperture Sizes

Some example aperture sizes were determined in a nonwoven two layer web of the present disclosure in the generally planar first region (as described herein) and in the plurality of discrete integral second regions (as described herein). The apertures in the generally planar first regions were considerable smaller than the apertures in the discrete integral second regions owing to the deformation process (e.g., FIG. 21). First the method measuring the apertures is described.

### Aperture Measurement Method Using High Resolution MicroCT

Sample Preparation and MicroCT Scanning
A 16 mm punch is used to physically extract a representative region of the two layer web. The 16 mm diameter sample is then placed in a sample holder with an inner diameter of 17 mm. The sample is packed in super low absorbing packing material to prevent motion during the scan. The sample holder is then placed in a Scanco mCT50 x-ray scanner (Scanco Medical, Zurich, Switzerland). The scanning was performed with an energy of 45 KeV, with 3000 projections and an integration time of 5 seconds per projection. The resulting data set is 5126×5126×1355 voxels with attenuation values represented as 16 bit integers. Each voxel has a diameter of 4 microns. The file is of a proprietary format and is referred to as the ISQ file in the following steps.

Image Visualization and Analysis
The objective of the image analysis is to measure the perceived area of apertures found in the sidewalls of the depressions of the scanned two layer web samples. The ISQ files described above, were read into Avizo 9.2.0 (FEI, Houston, Tex.). The data was resampled to 8 micron voxels for easier visualization through 3D volume rendering. Upon inspection of the 3D data, 3 different apertures were identified along the sidewalls of the depressions in the two layer web. For each of these apertures, a small subvolume was created and visualized with Avizo's Volume Rendering Module. A scalebar was also added to the image for reference. The camera position of the Volume Rendering was then adjusted so that it was normal to the aperture under inspection. The viewer was set to Orthographic mode so there would not be perspective distortion in the visualization. Once the best view of the aperture is obtained, a digital image of that view is created. In addition to those apertures in the sidewall, these steps were also repeated for apertures that were not in the sidewalls of the depression for comparison.
To measure the area of the apertures from the images, we employed software developed for P&G that allows exact web based measures to be made on images. The scale bar present in the image is used to calibrate lengths or areas measured in the images. A polygonal measuring tool is used to manually create a polygon around the perimeter of the apertures and allows automatic calculation of area and perimeter.

### Sample 1

Sample 1 was produced by first overbonding a 25 gsm PE/PP spunbond bicomponent layer, laminating that layer to a layer of 65 gsm carded, through-air bonded PE/PET nonwoven with a spiral glue pattern, and then passing the laminate through a pair of rolls, as illustrated in FIG. 21, at 0.135″ (3.38 mm) engagement of the rolls. The spunbond layer was against the male roll and the carded layer was against the female roll. The laminate may represent a topsheet and an acquisition layer in an absorbent article context. Note that the overbonds were only present in the spunbond bicomponent layer and not in the carded layer. The deformation process caused by the rolls of FIG. 21 induces strain into the laminate, which causes the overbonds to rupture and form apertures in only the spunbond layer. The amount of strain in the generally planar first regions is lower than the strain in the discrete integral second regions, which results in smaller apertures in the first regions and larger apertures in the discrete integral second regions. Smaller apertures in the first regions are desirable because smaller apertures feel softer to a wearer of an absorbent article having the laminate and are less likely to mark the skin of the wearer. Larger apertures in the discrete integral second regions are preferred because they may allow faster fluid acquisition in an absorbent article context, particularly with hydrophobic webs. Since the apertures in the discrete integral second regions do not come into contact with the skin in an absorbent article context, larger apertures in the discrete integral second regions will likely not negatively impact softness or mark the skin. Sample 1 had the nested embossing pattern of FIG. 70.

### Sample 2

Sample 2 was produced in the same way as Sample 1, except the spunbond bicomponent layer was ring rolled (e.g., FIG. 49) at 0.055″ (1.38 mm) engagement of the rolls after the spunbond layer was overbonded, and before it was laminated to the carded layer. The ring rolling process causes the overbonds to rupture and form small apertures in the spunbond layer. The strain induced by the rolls of FIG. 21 causes the apertures to become even larger. As with Sample 1, the apertures in the discrete integral second regions are significantly larger than those in the generally planar first regions. Sample 2 had the nested embossing pattern of FIG. 70.

| | Location of | No. Apertures | Average Area | Standard |
|---|---|---|---|---|
| | Aperture | Measured | (mm2) | deviation |
| Sample 1 | In discrete | 3 | 2.76 | 0.23 |
| | integral | | | |
| | second | | | |
| | regions | | | |
| Sample 1 | In generally | 4 | 0.84 | 0.36 |
| | planar first | | | |
| | region | | | |
| Sample 2 | In discrete | 3 | 2.86 | 0.12 |
| | integral | | | |
| | second | | | |
| | regions | | | |
| Sample 2 | In generally | 1 | 0.60 | n/a |
| | planar first | | | |
| | regions | | | |

### XI. Test Methods

A. Accelerated Compression Method.
1. Cut 10 samples of the specimen to be tested and 11 pieces of a paper towel into a 3 inch×3 inch (7.6 cm×7.6 cm) square.
2. Measure the caliper of each of the 10 specimens at 2.1 kPa and a dwell time of 2 seconds using a Thwing-Albert ProGage Thickness Tester or equivalent with a 50-60 millimeter diameter circular foot. Alternatively, a pressure of 0.5 kPa can be used. Record the pre-compression caliper to the nearest 0.01 mm.
3. Alternate the layers of the specimens to be tested with the pieces of paper towel, starting and ending with the paper towels. The choice of paper towel does not matter and is present to prevent “nesting” of the protrusions in the deformed samples. The samples should be oriented so the edges of each of the specimens and each of the paper towels are relatively aligned, and the protrusions in the specimens are all oriented the same direction.
4. Place the stack of samples into a 40±2° C. oven at 25±3% relative humidity and place a weight on top of the stack. The weight must be larger than the foot of the thickness tester. To simulate high pressures or low in-bag stack heights, apply 35 kPa (e.g. 17.5 kg weight over a 70×70 mm area). To simulate low pressures or high in-bag stack heights, apply 7.0 kPa (e.g. 3.4 kg weight over a 70×70 mm area), 4.0 kPa (e.g., 1.9 kg weight over a 70×70 mm area) of 1.0 kPa (e.g., 0.49 kg weight over a 70×70 mm area).
5. Leave the samples in the oven for 15 hours. After the time period has elapsed, remove the weight from the samples and remove the samples from the oven.
6. Within 30 minutes of removing the samples from the oven, measure the post-compression caliper as directed in step 2 above, making sure to maintain the same order in which the pre-compression caliper was recorded. Record the post-compression caliper of each of the 10 specimens to the nearest 0.01 mm.
7. Let the samples rest at 23±2° C. at 25±3% relative humidity for 24 hours without any weight on them.
8. After 24 hours, measure the post-recovery caliper of each of the 10 specimens as directed in step 2 above, making sure to maintain the same order in which the pre-compression and post-compression calipers were recorded. Record the post-recovery caliper of each of the 10 specimens to the nearest 0.01 mm. Calculate the amount of caliper recovery by subtracting the post-compression caliper from the post-recovery caliper and record to the nearest 0.01 mm.
9. If desired, an average of the 10 specimens can be calculated for the pre-compression, post-compression and post-recovery calipers.

B. Tensile Method

The MD and CD tensile properties are measured using World Strategic Partners (WSP) (harmonization of the two nonwovens organizations of INDA (North American based) and EDANA (Europe based)) Tensile Method 110.4 (05) Option B, with a 50 mm sample width, 60 mm gauge length, and 60 mm/min rate of extension. Note that the gauge length, rate of extension and resultant strain rate are from different from that specified within the method.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “90°” is intended to mean “about 90°”.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited in the Detailed Description are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by reference, the meaning or definition assigned to the term in this written document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A nonwoven material having a first surface and a second surface, the nonwoven material extending in an imaginary X-Y plane and comprising:
a plurality of fibers,
a generally planar first region; and
a plurality of discrete integral second regions that comprise deformations forming protrusions extending outward from the first surface of the nonwoven material and openings in the second surface of the nonwoven material, wherein the protrusions are formed from the fibers, wherein the protrusions have an exterior width, and two ends that define a length of the protrusions therebetween, and wherein the protrusions comprise a base proximate to the first surface of the nonwoven material, an opposed distal end extending outward in the Z-direction from the base, side walls between the base and the distal end of the protrusion, and a cap comprising at least a portion of the side walls and the distal end of the protrusion;
wherein the side walls have interior surfaces, wherein the interior surfaces of the side walls define a base opening at the base of the protrusion, wherein the cap has a portion with a maximum interior width, wherein the base opening has a width, and wherein the maximum interior width of the cap of the protrusions is greater than the width of the base opening; and
wherein a plurality of apertures are defined through the nonwoven material in portions of the generally planar first region or through portions of at least some of the plurality of discrete integral second regions, and wherein the apertures are formed in a predetermined, intentional pattern.

2. The nonwoven material of claim 1, wherein the exterior width of the protrusions varies along the length of the protrusions when viewed looking at the nonwoven material in a direction perpendicular to the X-Y plane of the nonwoven material.

3. The nonwoven material of claim 1, wherein the plurality of apertures are defined through only the portions of the generally planar first region.

4. The nonwoven material of claim 1, wherein the plurality of apertures are defined through only the at least some of the plurality of discrete integral second regions.

5. The nonwoven material of claim 1, wherein the plurality of apertures are defined at least partially through the side walls.

6. The nonwoven material of claim 1, wherein the plurality of apertures are defined at least partially through the distal ends.

7. The nonwoven material of claim 1, wherein the plurality of apertures are defined at least partially through the cap.

8. The nonwoven material of claim 1, wherein the plurality of apertures are defined through the portion of the generally planar first region and through the portions of at least some of the plurality of discrete integral second regions.

9. The nonwoven material of claim 1, wherein a perimeter of at least some of the plurality of apertures comprises a discontinuous melt lip.

10. The nonwoven material of claim 1, wherein a perimeter of at least some of the plurality of apertures comprises a densified region surrounding the at least some of the plurality of apertures.

11. The nonwoven material of claim 1, wherein the nonwoven material comprises a first layer and a second layer.

12. The nonwoven material of claim 11, wherein the plurality of apertures are defined through only the first layer or defined through only the second layer.

13. The nonwoven material of claim 11, wherein the plurality of apertures are defined through the first layer and the second layer.

14. The nonwoven material of claim 11, wherein the plurality of apertures are defined through the first layer and the second layer at coinciding locations in the first layer and the second layer.

15. An absorbent article comprising:
the nonwoven material of claim 11;
wherein the first layer forms a topsheet;
wherein the second layer forms an acquisition layer; and
an absorbent core;
wherein the protrusions extend towards the absorbent core; and
wherein the plurality of apertures are defined through only the topsheet.

16. An absorbent article comprising:
the nonwoven material of claim 11;
wherein the first layer forms a topsheet;
wherein the second layer forms an acquisition layer; and
an absorbent core;
wherein the protrusions extend towards the absorbent core; and
wherein the plurality of apertures are defined through only the acquisition layer.

17. An absorbent article comprising:
the nonwoven material of claim 11;
wherein the first layer forms a topsheet;
wherein the second layer forms an acquisition layer; and
wherein the plurality of apertures are defined through the topsheet and the acquisition layer at coinciding same locations in the first layer and the second layer.

18. An absorbent article comprising:
the nonwoven material of claim 11;
wherein the first layer forms a topsheet;
wherein the second layer forms an acquisition layer; and
a central lateral axis;
wherein the topsheet has a first width measured parallel to the central lateral axis;
wherein the acquisition layer has a second width measured parallel to the central lateral axis; and
wherein the first width is larger than the second width.

19. An absorbent article comprising:
the nonwoven material of claim 18;
wherein the first layer forms a topsheet;
wherein the second layer forms an acquisition layer; and
a central longitudinal axis;
wherein the topsheet has a first length measured parallel to the central longitudinal axis;
wherein the acquisition layer has a second length measured parallel to the central longitudinal axis; and
wherein the first length is larger than the second length.

20. An absorbent article comprising:
a liquid pervious nonwoven material having a first surface and a second surface, the nonwoven material extending in an imaginary X-Y plane and comprising a plurality of fibers, wherein the nonwoven material comprises a generally planar first region and a plurality of discrete integral second regions that comprise deformations forming protrusions extending outward from the first surface of the nonwoven material and openings in the second surface of the nonwoven material, the protrusions being formed from the fibers, wherein at least some of the protrusions have an exterior width, and two ends that define a length of the protrusion therebetween, wherein the at least some of the protrusions comprise a base proximate to the first surface of the nonwoven material, an opposed distal end extending outward in the Z-direction from the base, side walls between the base and the distal end of the protrusion, and a cap comprising at least a portion of the side walls and the distal end of the protrusion, wherein the side walls have interior surfaces, and wherein the exterior width of the protrusion varies along the length of the protrusion when viewed looking at the nonwoven material in a direction perpendicular to the X-Y plane of the nonwoven material;
wherein a plurality of apertures are defined through the nonwoven material in portions of the generally planar first region or through portions of at least some of the plurality of discrete integral second regions, and wherein the apertures form a repeating pattern;
a liquid impervious backsheet; and
an absorbent core positioned at least partially intermediate the liquid pervious nonwoven material and the backsheet.

21. The absorbent article of claim 20, wherein the liquid pervious nonwoven material comprises a topsheet and an acquisition layer, and wherein the apertures are defined through the portions of the generally planar first region and through the portions of at least some of the plurality of discrete integral second regions.

22. The absorbent article of claim 21, wherein the apertures in the portions of the generally planar first region have a size and/or shape that is different than the apertures in the portions of at least some of the plurality of discrete integral second regions.
